(19) United States
(12) Patent Application Publication
(10) Pub. No.: US 2025/0006313 A1
(43) Pub. Date: Jan. 2, 2025

(54) HIGH-THROUGHPUT PREDICTION OF VARIANT EFFECTS FROM CONFORMATIONAL DYNAMICS

(71) Applicant: Invitae Corporation, San Francisco, CA (US)

(72) Inventors: John Michael NICOLUDIS, San Francisco, CA (US); Carlos L. ARAYA, Montecito, CA (US); Toby MANDERS, San Francisco, CA (US); Alexandre COLAVIN, San Diego, CA (US); Gert KISS, Santa Clara, CA (US)

(73) Assignee: Invitae Corporation, San Francisco, CA (US)

(21) Appl. No.: 18/700,911

(22) PCT Filed: Oct. 13, 2022
(86) PCT No.: PCT/US2022/078070

§ 371 Date: Apr. 12, 2024

Related U.S. Application Data
(60) Provisional application No. 63/262,484, filed on Oct. 13, 2021.

(51) Int. Cl. G16C 20/30; G16C 20/70

(52) U.S. Cl. CPC G16C 20/30; G16C 20/70

(57) ABSTRACT

The present disclosure provides methods for automatically predicting the functional significance and clinical interpretation of variants (e.g., protein missense variants such as mutations) of unknown significance observed, e.g., in medical genetic testing, using the conformational dynamics of molecular structures (e.g., protein structures). The disclosure provides computer implemented methods, and integrated data, infrastructure, and software systems that can generate conformational dynamics (e.g., using molecular dynamics) of protein structures, compute features from these simulations, extract conformational states, initiate simulations for relevant variants (e.g., missense variants), and train, test, and deploy machine learning models for scoring the clinical significance of the variants.

### FIELD

The present disclosure relates to methods for determining the functional effects of genetic variation, e.g., protein missense mutations, using machine learning on the conformational dynamics of biological molecules.

### BACKGROUND

Accurate variant effect prediction is critically important in genetic studies and clinical diagnosis. Missense variants are the most common type of coding genetic variants and are a major class of genetic risk across a broad range of common and rare diseases. There is a substantial contribution from de novo missense mutations to structural birth defects (Homsy et al. (2015) Science 350:1262-1266; Jin et al. (2017) Nat. Genet. 49: 1593-1601) and neurodevelopmental disorders (Iossifov et al. (2014) Nature 514:216-221; De Rubeis et al. (2014) Nature 515:209-215; McRae et al. (2017) Nature 542: 433-438). Nevertheless, only a small fraction of missense de novo mutations is pathogenic and will cause disease. In clinical genetic testing, many missense variants in well-established risk genes are classified as variants of uncertain significance, indicating that the current clinical information known is not sufficient to determine whether the variant is pathogenic or benign.

Interpretation of the molecular-level effect of genetic variants (e.g., missense gene variants) remains challenging and requires a particular investigation of mutation (either at the amino acid or nucleic acid level) in the context of molecular structure and function. Given that the damaging and/or neutral consequences of genetic variants are reportedly associated with the structural properties of the affected molecules (Sivley et al. (2018) Am. J. Hum. Genet. 102:415-426; Hicks et al. (2019) Proc. Natl. Acad. Sci. U.S.A. 116: 8960-8965), resources and tools have been developed to map and visualize variants (e.g., missense variants) on molecular structures (e.g., protein structures), such as ASTRID, mutation3D, COSMIC-3D, VarSite, VarMap, or PhyreRisk. However, estimating the biological effect of a mutation from mere visualization is speculative at best. Current high-throughput methods that use structural properties are able to predict changes in protein structure that are typically associated with changes in the overall stability of the protein, which is the mechanism by which many loss-of-function (LOF) missense mutations are pathogenic. However, there is less known about how the changes in conformational dynamics of molecular structures relate to pathogenicity. Thus, determining variants' effect on conformational dynamics and correlating the observed dynamics with pathogenicity represents a growing research area. Molecular dynamics (MD) methods, which are computational algorithms to determine the conformational dynamics of molecular structures, have been used, for example, in the methods described in U.S. Publ. No. US20190189243A1 and McCoy et al. (2020) Biophysical Journal 120 (2): 189-204. However, the methods currently known in the art are not scalable, and therefore cannot operate in a true high throughput mode, as these methods rely on human curation to identify and select features for machine learning, and the resulting predictive models are specific for a particular biological system (e.g., a particular protein related to a disease or disorder). Furthermore, the computational algorithms used to determine the conformational dynamics of a molecular structure are currently prohibitively time-consuming to be used for clinical variant effect prediction in a scalable genetic testing system.

### BRIEF SUMMARY

The present disclosure provides a computer implemented method for high throughput classification of variant effects comprising (i) generating conformational dynamics (e.g. using molecular dynamics (MD)) of a reference molecule structure; (ii) processing the conformational dynamics of the reference molecule structure to generate a low-dimensional representation of the conformational landscape of the reference molecule structure; (iii) extracting a metastable conformational state from the low-dimensional conformational landscape of the reference molecule structure to seed simulations of a variant molecule structure; (iv) generating a variant molecule structure or set thereof by introducing at least one variant in the extracted conformational state of the reference molecule structure; (v) generating conformational dynamics of the variant molecule structure or set thereof (e.g. using MD); (vi) processing the conformational dynamics of the variant molecule structure or set thereof to generate a low-dimensional representation of the conformational landscape of the variant molecule structure or set thereof; and, (vii) training a machine-learning predictive model using the low-dimensional representation of the conformational landscape of the reference molecule structure and variant molecule structures using clinical data as training labels, wherein the predictive model classifies the variant effects in the reference molecule structure. In some aspects, generating conformational dynamics of the reference molecule structure or variant molecule structure comprises (a) retrieving a reference molecule structure or variant molecule structure; and, (b) generating conformational dynamics using the retrieved reference molecule structure or variant protein structure. In some aspects, the reference molecule structure or variant molecule structure are retrieved from the Protein Data Bank (PDB) or AlphaFold. In some aspects, the conformational dynamics are generated using GROMACS, OpenMM, NAMD, Amber or LAMPS.

In some aspects, the generation of conformational dynamics is parallelized. In some aspects, the reference molecule structure is a three dimensional structure of a wild type protein. In some aspects, each one of step (i) and/or step (ii) of the method disclosed above comprises independently at least about 10,000, at least about 20,000, at least about 30,000, at least about 40,000, at least about 50,000, at least about 60,000, at least about 70,000, at least about 80,000, at least about 90,000, at least about 100,000, at least about 200,000, at least about 300,000, at least about 400,000, at least about 500,000, at least about 600,000, at least about 700,000, at least about 800,000, at least about 900,000, or at least about 1,000,000 simulated structural conformations that comprise the conformational dynamics.

In some aspects, processing the conformational dynamics to generate a low-dimensional representation of the conformational landscape of the reference molecule structure comprises extracting structural features from the conformational dynamics. In some aspects, the structural features are selected from the group consisting of bond angles, inter-residue distances, residue fluctuation, surface accessibility, domain relative orientations, binding-site interactions, and any combination thereof. In some aspects, the extraction of structural features results in at least a 20-fold, 30-fold, 40-fold, or 50-fold reduction in structural features.

In some aspects, processing the conformational dynamics further comprises dimensionality reduction. In some aspects the dimensionality reduction is obtained using principal component analysis (PCA) or autoencoders. In some aspects, the dimensionality reduction results in at least a 50-fold, 60-fold, 70-fold, 80-fold, 90-fold or 100-fold reduction in structural features.

In some aspects, the low-dimensional representation of the conformational landscape identifies conformations in which the reference molecule structure and/or variant molecule structure is in a metastable conformational state. In some aspects, the low-dimensional representation comprises modes of movement that characterize conformational changes that are relevant to molecular function.

In some aspects, extracting a metastable conformational state from the low-dimensional conformational landscape comprises clustering conformational states based on their representation in the low-dimensional space. In some aspects, clustering conformational states comprises k-mean clustering, hierarchical clustering, density-based clustering, DBSCAN, spectral clustering, Gaussian mixture models, or any combination thereof. In some aspects, the method further comprises prioritizing the clustered conformational states. In some aspects, prioritizing clustered conformational states is based on cluster properties. In some aspects, the cluster properties are selected from the group consisting of distance from conformational landscape centroid, cluster occupancy, cluster distribution statistics, structural properties of the cluster, thermodynamics of cluster, or any combination thereof. In some aspects, the structural properties are selected from the group consisting of bond angles, inter-residue distances, surface accessibility, domain relative orientations, binding-site interactions, structural similarity to known protein conformations, and any combination thereof. In some aspects, the bond angles comprise phi-psi dihedrals, side chain chi angles, or combinations thereof. In some aspects, the cluster distribution statistics are selected from the group consisting of Silhouette score, elbow score, Calinski-Harabasz Index, Rand Index, mutual information, homogeneity, completeness, V measure, Davies-Bouldin Index, and any combination thereof.

In some aspects, extracting a metastable conformational state from the low-dimensional conformational landscape comprises extracting a representative conformational state. In some aspects, the representative conformational state is a conformational state that is amongst the closest to the centroid of the all the conformational states of a cluster. In some aspects, at least one variant is a pathogenic mutation. In some aspects, at least one variant is a non-pathogenic (benign) mutation. In some aspects, the variant molecule structure or set thereof comprises a variant observed or that could be observed in medical genetic testing. In some aspects, the variant molecule structure or set thereof comprises a pathogenic variant. In some aspects, the variant molecule structure or set thereof comprises a non-pathogenic (benign) variant. In some aspects, the variant molecule structure or set thereof comprises a variant of unknown significance. In some aspects, the variant molecule structure or set thereof is generated using a backbone-dependent rotamer library. In some aspects, the method comprises identifying sidechain conformations (rotamers) with the existing backbone dihedral angles of the variant molecule structure at the variant position in the backbone-dependent rotamer library. In some aspects, the method further comprises testing whether the introduction of a rotamer identified backbone-dependent rotamer library causes a steric clash with nearby residues. In some aspects, testing is conducted until a rotamer is identified that minimizes steric clash. In some aspects, the method further comprises conducting a minimization and equilibration simulation to regularize the geometry of the conformation of the variant molecule structure and decrease the energetic impact of introducing the variant in the variant molecule structure.

In some aspects, the predictive model is generated using machine-learning. In some aspects, the clinical data used as training labels comprises biomarker status (e.g., presence or absence of a certain biomarker or its expression level), biometric data, lifestyle-related data, response to treatments, symptoms of the disease or conditions, protein expression data, type of treatment administered, dosage, dosage regimen, administration route, presence or absence of co-therapies, response to the therapy, age, body weight, gender, ethnicity, ClinVar submissions, INVITAE™ clinical interpretations, data from other experimental or computational models, or any combination thereof. In some aspects, the classification of the variant effect in the reference molecule structure comprises the calculation of a predicted pathogenicity probability. In some aspects, the machine-learning predictive model is generated using Logistic Regression. In some aspects, the machine-learning predictive model is generated using Random Forests. In some aspects, the machine-learning predictive model is generated using an Artificial Neural Network.

The present disclosure also provides a computer implemented method for scoring the clinical significance of a variant comprising (a) generating conformational dynamics (e.g., using VID) of a reference molecule structure; (b) processing the conformational dynamics of the reference molecule structure to generate a low-dimensional representation of the conformational landscape of the reference molecule structure; (c) extracting a metastable conformational state from the low-dimensional conformational landscape of the reference molecule structure to seed simulations of a variant molecule structure; (d) generating a variant molecule structure or set thereof by introducing at least one variant in the extracted metastable conformational state of the reference molecule structure; (e) generating conformational dynamics of the variant molecule structure or set thereof (e.g. using MD); (f) processing the conformational dynamics of the variant molecule structure or set thereof to generate a low-dimensional representation of the conformational landscape of the variant molecule structure or set thereof; and, (g) training a machine-learning predictive model using the low-dimensional representation of the conformational landscape of the reference molecule structure and variant molecule structures using clinical data as training labels, wherein the predictive model outputs a score the clinical significance of the variant.

The present disclosure also provides a system comprising (a) a memory; and, (b) at least one processor coupled to the memory and configured for (i) generating conformational dynamics of a reference molecule structure (e.g. using MD); (ii) processing the conformational dynamics of the reference molecule structure to generate a low-dimensional representation of the conformational landscape of the reference molecule structure; (iii) extracting a metastable conformational state from the low-dimensional conformational landscape of the reference molecule structure to seed simulations of a variant molecule structure; (iv) generating a variant molecule structure or set thereof by introducing at least one variant in the extracted metastable conformational state of the reference molecule structure; (v) generating conformational dynamics of the variant molecule structure or set thereof (e.g. using MD); (vi) processing the conformational dynamics of the variant molecule structure or set thereof to generate a low-dimensional representation of the conformational landscape of the variant molecule structure or set thereof, and, (vii) training a machine-learning predictive model using the low-dimensional representation of the conformational landscape of the reference molecule structure and variant molecule structures using clinical data as training labels, wherein the predictive model classifies the variant effects in the reference molecule structure.

The present disclosure also provides tangible computer readable device having instruction stored thereon that, when executed by at least one computing device, causes the at least one computing device to perform operations comprising (i) generating conformational dynamics (e.g. using MD) of a reference molecule structure; (ii) processing the conformational dynamics of the reference molecule structure to generate a low-dimensional representation of the conformational landscape of the reference molecule structure; (iii) extracting a metastable conformational state from the low-dimensional conformational landscape of the reference molecule structure to seed simulations of a variant molecule structure; (iv) generating a variant molecule structure or set thereof by introducing at least one variant in the extracted metastable conformational state of the reference protein structure; (v) generating conformational dynamics of the variant molecule structure or set thereof (e.g. using MD); (vi) processing the conformational dynamics of the variant molecule structure or set thereof to generate a low-dimensional representation of the conformational landscape of the variant molecule structure or set thereof, and, (vii) training a machine-learning predictive model using the low-dimensional representation of the conformational landscape of the reference molecule structure and variant molecule structures using clinical data as training labels, wherein the predictive model classifies the variant effects in the reference molecule structure.

In some aspects, of the methods, systems, or tangible computable readable devices disclosed above, the classification of the variant in the variant molecule structure can be used to (i) treat a patient, (ii) selected a patient for treatment, (iii) commence a treatment, (iv) discontinue a treatment, (v) interrupt a treatment, (vi) modify a treatment, or (vii) any combination thereof.

In some aspects, the present disclosure provides a method to (i) treat a patient, (ii) selected a patient for treatment, (iii) commence a treatment, (iv) discontinue a treatment, (v) interrupt a treatment, (vi) modify a treatment, or (vii) any combination thereof, comprising applying the any of the methods, systems, or tangible computable readable devices disclosed above.

The present disclosure also provides a personalized medicine treatment comprising a therapeutic agent that can treat a disease or disorder caused by a variant identified as pathogenic by any of the methods disclosed above, or by using any of the systems or the tangible computable readable devices disclosed above.

The present disclosure also provides a computer implemented method for generating a low-dimensional representation of the conformation landscape of a molecule structure comprising (i) extracting structural features from the conformational landscape of the molecule structure, and (ii) reducing the dimensionality of the conformational landscape. In some aspects, the structural features are selected from the group consisting of bond angles, inter-residue distances, residue fluctuation, surface accessibility, domain relative orientations, binding-site interactions, and any combination thereof. In some aspects, extracting structural features results in at least a 20-fold, 30-fold, 40-fold, or 50-fold reduction in structural features. In some aspects, reducing the dimensionality comprises using principal component analysis (PCA) or autoencoders. In some aspects, reducing the dimensionality results in at least a 50-fold, 60-fold, 70-fold, 80-fold, 90-fold or 100-fold reduction in structural features.

The present disclosure also provides a computer implemented method for identifying metastable conformational states of a molecule structure comprising clustering low-dimensional representations of the conformational landscape of the molecule structure. In some aspects, clustering conformational states comprises k-mean clustering, hierarchical clustering, density-based clustering, DBSCAN, spectral clustering, Gaussian mixture models, or any combination thereof. In some aspects, the method further comprises prioritizing the clustered conformational states. In some aspects, prioritizing clustered conformational states is based on cluster properties. In some aspects, the cluster properties are selected from the group consisting of distance from conformational landscape centroid, cluster occupancy, cluster distribution statistics, structural properties of the cluster, thermodynamics of cluster, or any combination thereof. In some aspects, the structural properties are selected from the group consisting of bond angles, inter-residue distances, surface accessibility, domain relative orientations, binding-site interactions, structural similarity to known protein conformations, and any combination thereof. In some aspects, the bond angles comprise phi-psi dihedrals, side chain chi angles, or combinations thereof. In some aspects, the cluster distribution statistics are selected from the group consisting of Silhouette score, elbow score, Calinski-Harabasz Index, Rand Index, mutual information, homogeneity, completeness, V measure, Davies-Bouldin Index, and any combination thereof.

The present disclosure also provides a computer implemented method for high throughput classification of variant effects in a molecule structure comprising (i) generating a low-dimensional representation of the conformation landscape of the molecule structure, and (ii) identifying metastable conformational states from the low-dimensional representation of the conformation landscape of the molecule structure. In some aspects, generating a low-dimensional representation of the conformation landscape of the molecule comprises (a) extracting structural features from the conformation landscape of the molecule structure, and (b) reducing the dimensionality of the conformational landscape. In some aspects, identifying metastable conformational states from the low-dimensional representation of the conformation landscape of the molecule structure comprises clustering low-dimensional representations of the conformation landscape of the molecule structure.

### BRIEF DESCRIPTION OF THE DRAWINGS/FIGURES

FIG. 1 shows a system diagram for the predictive system of the present disclosure.

FIG. 2 shows a workflow diagram illustrating the sequential steps in the predictive system to predict variant effects from the conformational dynamics of biological molecules.

FIG. 3 shows a schematic view of the dimensionality reduction portion of the workflow, including a structural feature extraction step and a dimensional reduction step.

FIG. 4 shows a schematic representation of the energetic state of the conformation of a reference molecule and the energetic states of metastable conformations of variant molecules.

FIG. 5 exemplifies that there is a relationship between features such as backbone psi-angle distribution and pathogenicity of a variant molecule.

FIG. 6 shows a low dimensional conformation landscape representation resulting from clustering >76,000 structural conformations from conformational dynamics of a reference molecule which have been subjected to structural feature extraction and dimensionality reduction. This low-dimensional representation is then subjected to clustering. Labels identify specific conformational clusters, each one of which represent a metastable conformation of the reference molecule. Component 1 and component two are the first two representative components from principal component analysis.

FIG. 7 shows that conformational clusters can be used to distinguish pathogenic and benign variants (e.g., missense gene variants).

FIG. 8 shows the various amounts of conformational data (dataset definition) and featurization methods used to optimize a machine-learning model. For each combination of dataset and featurization methods (except where they were not computed: NC), a machine-learning model is trained on the features extracted from the simulations included in the dataset definition. The performance of the model is evaluated using five-fold cross-validation over the training set to determine the best performing model according to a balance-weighted F1 score. The performance of the final model (in this case, dataset “3 ns, 2 repetitions, 11 states” and featurization method “connectedness tICA”) is then determined using the balance-weighted F1 score of the hold-out test set of simulations that were not used in initial model performance and evaluation.

FIG. 9 shows the performance of the model (LSS model) over a selection of variants compared to the performance of the same variants in a sequence-based model and a FoldX-based model and compared to the true label. Overall the LSS model predictions for these variants are closer to the true labels than any other model.

FIG. 10 compares the performance of a predictive model for the MSH2 (MutS Homolog 2) tumor suppressor developed according to the methods disclosed herein (LSS) compared to alternative methods, e.g., FoldX and Polyphen.

### DETAILED DESCRIPTION

The disclosure provides a true high-throughput platform that can accurately predict the phenotype, phenotypic effect, or biological effect (e.g., whether they are pathogenic and their severity of the pathological phenotype) of variants (e.g., missense variants) using the conformational dynamics (e.g., generated from molecular dynamics) of molecular structures (e.g., protein structures), which is agnostic to a particular biological system, and eliminates the need for biological and structural biological expertise to train a machine learning model

These insights aid in the interpretation of variants of unknown significance from medical genetic testing and can guide pharmacological interventions using the principles of precision medicine.

In contrast, prior variant (e.g., missense variant) classification systems in the art are incapable of being true high-throughput platforms that can accurately predict variant effects from conformational dynamics, and such systems typically require biological and structural biological expertise to train the machine learning models. Aspects of the present disclosure achieve better performance and throughput than other methods in the art by featurizating and reducing the dimensionality of conformational dynamics in a system agnostic manner. Thus, the need for biological and structural biological human expertise to train a machine-learning model is eliminated. Furthermore, the process of determining metastable conformational states from the reference molecular system, determining conformational dynamics of variants introduced into these states, and training a machine-learning model on the low-dimensional representation of these states greatly reduces the amount of simulation time required to train an accurate model, thereby increasing the throughput of this approach in a variant interpretation system. Lastly, this approach provides a way to make variant effect predictions quickly on newly observed variants by storing pre-trained models for inference.

The present disclosure provides computer implemented methods for high throughput prediction of biological effects of variants (e.g., missense gene variants) from conformational dynamics of molecular structures, the methods comprising, e.g., (a) generating conformational dynamics of a reference molecule (e.g., a reference protein); (b) using unsupervised dimensionality reduction methods to define a low-dimensional conformational landscape of the reference molecule; (c) determining a metastable conformational state or set thereof from the low-dimensional conformational landscape of the reference molecule; (d) constructing a variant molecule (e.g., a variant protein) or set thereof by introducing at least one molecular alteration to the reference molecule in a metastable conformational state or set thereof; (e) determining the conformational dynamics of the variant molecule or set thereof in the metastable conformational state or set thereof; (f) using unsupervised dimensionality reduction methods to generate a low-dimensional representation of conformational dynamics of the variant molecule or set thereof in the metastable conformational state or set thereof; (g) training a predictive model using the low-dimensional representation of the conformational dynamics of the variant molecule or set thereof using biological or clinical data as training labels, wherein the model predicts biological effects of variants (e.g., missense mutations) on the reference molecule; and optionally (h) extending the predictive model to newly observed variants from medical genetic testing expeditiously.

In order to generate conformational dynamics of a molecular structure (e.g., a protein structure), the molecular structure must be defined within a molecular system.

As used herein, a “molecular system” refers to a collection of molecular structures of biological molecules such as a protein, peptide, nucleic acid or other biologically-relevant molecule (e.g. lipids, glycans, or small molecules). The molecular system can also include solvents or other molecules (e.g. ions, lipids) that define a realistic biological composition of the molecular components in the system. The “molecular system” comprises a collection of molecular components that integrate the system (e.g., protein, solvation water, ions, etc.), while a “conformational state” of the molecular system is a three-dimensional arrangement of the molecular components of the molecular system at a certain point in time. Thus, a conformational state represents a ‘snapshot’ of a molecular system. As used herein, the terms “conformation,” “conformational structure,” “structure,” and “molecular structure,” can be used interchangeably with “conformational state.”

It is understood that a reference molecule does not adopt a structure in a vacuum, and therefore, additional components of the system (e.g., water molecules, ions, etc.) must be present. Accordingly, in the context of the present disclosure, references to a “reference molecule structure” or to a “variant molecule structure” imply the presence of those additional components. Accordingly, the terms “reference molecular system” and “reference molecule structure” are considered equivalent and interchangeable. Likewise, the terms “variant molecular system” and “variant molecule structure” are considered equivalent and interchangeable.

As used herein, the terms “molecular structure,” “molecule structure,” and grammatical variants thereof refer to the three-dimensional coordinates and identities of the atoms that make up some or all of a biological molecule (e.g., a protein) or set thereof and the interactions (e.g. covalent and non-covalent bonds) between those atoms. The molecular structure can be represented, e.g., by ‘coarse-graining’ methods (such as the MARTINI algorithm) that decrease the complexity of a molecular structure by replacing set(s) of atoms with ‘dummy’ atoms with properties meant to represent a combination of atoms (e.g. an amino acid) and their interactions with other atoms.

A molecular system can be generated, for example, by retrieving molecular structures (e.g., crystallographic protein structures) from databases of experimental structures, e.g., Protein Data Bank (PDB) or Electron Microscopy Data Bank (EMDB), determining the molecular structures independently through experimental or computational methods and combining them into one or more data files, or retrieving molecular structures from existing databases of computationally predicted structures, such as the ModBase database or the European Biological Institute database of AlphaFold predicted structures. Alternatively, molecular structures can be predicted using protein structure prediction algorithms such as EVFold, AlphaFold and RoseTTAFold, etc., or generated by homology modeling. Molecular structures can be further informed by experimental methods (e.g. single-molecule biophysics, large-scale mutagenesis experiments (e.g. deep mutational scanning), mass spectrometry). Molecular structures can also be determined through any combination of the above in an approach called integrative modeling.

As used herein, the term “molecule” refers to biological entities such as proteins, lipids, nucleic acids, carbohydrates, and combinations thereof. The term molecule as used herein encompasses both single molecular entities and a plurality of them. Accordingly, in some aspects, a molecule as defined herein can be, e.g., a multimeric complex comprising multiple protein subunits, or proteins complexed with nucleic acids, etc. In some aspects, the term molecule refers to a biological molecule such as a protein or a nucleic acid complexed with a ligand, e.g., an antagonist or agonist of a receptor, a cofactor, a drug, etc. Particular types of molecules according to the present disclosure are reference molecules and variant molecules. “Molecule” refers to the composition of an entity, while “conformation” or “structure” refer to the configuration of the molecule in space.

As used herein, the term “reference molecule” refers to the molecule (e.g., a protein) to be investigated by the introduction of variants (e.g., missense variants). A reference molecule (e.g., a protein) can be the native protein (e.g. wild-type protein). Thus, in some aspects, the reference molecule structure can be the structure of a native protein (e.g. wild-type protein) or set thereof. In other aspects, instead of being the wild-type form, the reference molecule (e.g., a reference protein) can contain a common or uncommon variant (e.g., a missense variant). Thus, in other aspects, the reference molecule structure can be the structure of a variant molecule (e.g., a missense variant protein) or set thereof. Accordingly, the reference molecule (e.g., a reference protein) is not required to be free of genetic variants, but is to be used as a reference molecule to compare to a set of other variant molecules with the same background system, i.e., a variant molecule (e.g., a variant protein) may be used as a reference molecule that determines the effect of variants (e.g., mutations) in a set of other variant molecules (e.g., other variant proteins with different sets of mutations).

A reference molecule is defined by its composition (the identities and relationships of atoms in the molecule). The methods disclosed herein comprise retrieving a structure (reference molecular structure) from a database, which represents the reference molecule in a particular conformational state. Methods like MD can be used to generate conformational dynamics of the reference molecule. These conformational dynamics approximate the conformational landscape of the reference molecule. A conformational landscape is the collection of energetically-accessible (given the parameters i.e. temperature, pressure of the system) conformational states for a reference molecule.

The terms “reference molecular structure,” “reference molecule structure,” and grammatical variants thereof can refer to a single conformational state of the structure of the reference molecule (e.g., a reference protein) or to an ensemble of conformational states of the structure of the reference molecule.

The term “variant” as used herein refers to a particular change in a molecule (e.g., a protein or gene) relative to an established reference molecule. For example, a variant can be a change in a nucleic acid (e.g., a gene), a protein, a lipid, a carbohydrate, etc., with respect to a reference molecule, i.e., the corresponding molecule without the change. In some aspects, the variant is a gene variant. As used herein the term “variant molecule” refers to the molecule (e.g., a protein) comprising at least one variant (e.g., a missense variant). As used herein, the terms “variant molecular structure,” “variant molecule structure,” and grammatical variants thereof refer to the three-dimensional structure of a variant molecule. In some, as variant molecule structure can refer to a single conformational state of the structure of a variant molecule (e.g., a variant protein) or to an ensemble of conformational states of the structure of a variant molecule.

The term “gene” is used broadly and refers to any segment of DNA or other nucleic acid associated with biological function (e.g., an mRNA, a miRNA, etc.). Thus, the gene includes coding sequences, and regulatory sequences necessary for its expression. The gene also optionally includes non-expressed nucleic acid segments that, for example, form recognition sequences for other proteins. The gene may be derived from a variety of sources, including cloning from a source of interest, or synthesis from known or predicted sequence information, and may include sequences designed to have the desired parameters.

The term “gene variant” means any particular change in a nucleotide or nucleotide sequence relative to an established reference nucleotide or nucleotide sequence. The variation in the genomic information is useful to identify because it may be indicative of the cause of phenotypic variation among subjects—one theory being that invariant regions of the genomes of normal subjects are likely important for coding essential components necessary for the development and survival of those subjects. The variants may account for the normal phenotypic differences between subjects and/or the variants may account for disease-related variations. In some aspects, the variant is a mutant. A mutant is a polypeptide or polynucleotide sequence that has been altered by one or more substitutions, insertions, crosses, deletions, and/or other gene manipulations. For purposes of this disclosure, mutants and variants are not limited to any particular method of generating them. In some aspects, the mutant or variant sequence has increased, decreased, or substantially similar activity or characteristics compared to the parental sequence. In some aspects, variant polypeptides comprise one or more amino acid residues that are mutated in comparison to the amino acid sequence of a wild-type polypeptide (e.g., a parent polypeptide). In some aspects, one or more amino acid residues of the polypeptide among the variant polypeptides comprising the plurality of polypeptides remain constant, non-variant, or non-mutated relative to the parent polypeptide. In some aspects, the parent polypeptide is used as a basis for generating variants with improved stability, activity, or any other desired property.

In some aspects, the variant is a missense variant gene. As used herein, the term “missense variant gene” refers to a gene in which a nucleotide substitution results in the replacement of one amino acid with another in the protein made from the gene. The amino acid change may alter the function of the protein. In some aspects, the amino acid change can result in a pathogenic variant. In some aspects, the amino acid change can result in a benign variant.

The terms “protein,” “polypeptide”, and “peptide” are used interchangeably and refer to any polymer consisting of two or more amino acids covalently linked by an amide bond. In some cases, the polymer has at least about 30 amino acid residues, and generally at least about 50 amino acid residues. More typically, the polymer contains at least about 100 amino acid residues. The present disclosure is not intended to be limited to amino acid sequences of any particular length. The term generally encompasses a composition that is considered to be a fragment of a full-length protein or peptide. D- and L-amino acids, and mixtures of D- and L-amino acids are also included in this definition. The polypeptides described herein are not limited to amino acids that are genetically encoded. Indeed, in addition to the genetically encoded amino acids, the polypeptides described herein may consist entirely or partially of naturally occurring and/or synthetic noncoding amino acids. In some aspects, the polypeptide has an amino acid sequence, including amino acid addition or deletion (e.g., gap) and/or substitution, as compared to the amino acid sequence of the full-length parent polypeptide, while retaining the functional activity of the parent polypeptide. In some aspects, the polypeptide can be modified via post-translational modification (e.g. methylation, phosphorylation, glycosylation)

In some aspects, the variant can be a missense variant protein. As used herein, the term “missense variant protein” refers to a protein encoded by a missense variant gene, wherein the protein has an amino acid substitution with respect to a reference protein, e.g., the wild type protein or a variant protein that is prevalent in a population. A missense variant protein has a different amino acid sequence with respect to wild type or other reference protein and the change in the protein's sequence can affect both its physical and/or functional properties. Sometimes a single amino acid mutation in a protein can give rise to an inactive protein, which is a drastic change in functional activity and can cause a fatal disease in a subject. In some aspects, a substitution in a missense variant protein can have a biological effect, for example, causing a subject to develop a disease or disorder.

As used herein, the term “biological effect” includes the molecular effects of a given biological entity, e.g., a variant, wherein the variant is a protein such as a missense variant protein, as well as the effects of such entity at the level of a cell, tissue, or organism. Biological effect encompass, e.g., effects at the molecular level, as well as physiological effects. Thus, in some aspects, a change in the molecular structure (e.g., three-dimensional structure) of a protein or an alteration in its binding affinity for a ligand is considered a biological effect. In some aspects, a gain or loss of function of a protein can be considered a biological effect. In some aspects, the development of a disease or disorder due, e.g., to a gain or loss of protein function, or protein aggregation due to protein misfolding, can be considered a biological effect. In some aspects, the increase or reduction of the risk of developing a disease or disorder can be considered a biological effect. In some aspects, the manifestation of a pathological phenotype can be considered a biological effect. In some aspects, the terms “biological effect” and “clinical significance” can be used interchangeably.

Generating “conformational dynamics” of a reference molecule (e.g., a reference protein) involves using computational algorithms to determine conformational changes in the molecular structure of the reference molecule (e.g., a reference protein) or a variant molecule (e.g., a variant protein). In some aspects, these algorithms are called Molecular Dynamics (MD) simulations where the reference molecular system is simulated over time by completing computational cycles that involve, e.g., (1) computing the physical interaction energies between atoms and (2) displacing the atoms in accordance to these interaction energies. These simulation cycles, also known as steps, generate a trajectory of the reference molecule (e.g., a reference protein) or variant molecule (e.g., a variant protein) over time, characterizing the conformational dynamics of the reference molecule or a variant molecule. As used herein, the term “trajectory” refers to a set of data resulting from a conformational dynamic simulation that contains discrete conformational states that the reference molecule or variant molecule adopts during the simulation over a specific period of time.

In some aspects, at least one (1) independent trajectory can characterize the conformational dynamics of the reference molecule or variant molecule. In some aspects, the MD simulations are conducted using GROMACS, OpenMM, NAMD, Amber or LAMMPS. In some aspects, the sampling of MD simulations can be improved by using advanced sampling (AS) methods, e.g. umbrella sampling, parallel tempering, replica exchange, metadynamics, and simulated annealing. In some aspects, deep-learning algorithms can be used to predict the conformational dynamics of a reference molecule (e.g., a reference protein) or a variant molecule (e.g., a variant protein).

In some aspects, multiple conformational states of the same reference molecule (e.g., a reference protein) or a variant molecule (e.g., a variant protein) or their respective molecular structures can be used to infer the conformational dynamics of the reference molecule (e.g., a reference protein) or the variant molecule (e.g., a variant protein). The conformational dynamics of a reference molecule (e.g., a reference protein) or a variant molecule (e.g., a variant protein) can comprises, e.g., from about 10,000 to about 1,000,000 or more conformational states of the reference molecule (e.g., a reference protein) or a variant molecule (e.g., a variant protein). The conformational dynamics of a reference molecule structure (e.g., a reference protein structure) or a variant molecule structure (e.g., a variant protein structure) can include a generative model that can be used to generate conformational states according to a distribution that represents the conformational dynamics.

In the context of the present disclosure, the term “low-dimensional conformational landscape” refers to a plurality of conformational states of the conformational dynamics of a reference molecule (e.g., a reference protein) or a variant molecule (e.g., a variant protein) resulting from the application of dimensionality reduction. Generating a low-dimensional conformational landscape of the conformational dynamics of the molecule (e.g., a reference molecule or variant molecule, for example, a reference protein or a variant protein) can comprise, e.g., (1) applying molecular featurization methods to the conformational states that define the conformational dynamics of the molecule, and (2) using unsupervised dimensionality reduction methods to these featurized conformational states to further reduce the dimensionality.

A low-dimensional conformational landscape can reveal relevant conformational dynamics that are involved in the function and/or behavior of the components (e.g., atoms) of a reference or variant molecule (e.g., a reference protein or a variant protein). The conformational landscape of a reference or variant molecule (e.g., a reference protein or a variant protein) can be represented, e.g., as a discrete distribution of individual conformational states from conformational dynamics trajectories in the low-dimensional space. The conformational landscape can also be represented as a continuous distribution in the low-dimensional space. The density of the distribution reflects the tendency of a reference or variant molecule (e.g., a reference protein or a variant protein) to occupy a particular region in the conformational landscape, and in some aspects, this density correlates with the overall energy of the reference or variant molecule (e.g., a reference protein or a variant protein) such that highly dense areas of the conformational landscape reflect low energy conformations. The conformational dynamics of variant molecules (e.g., variant proteins) containing pathogenic molecular alterations (variants or mutations) may be distinct from the conformational dynamics of the reference molecule (e.g., a reference protein) or a variant molecule (e.g., a variant protein) with benign molecular alterations (variants or mutations).

The methods disclosed herein comprise a featurization or feature extraction step. As used herein, the terms “featurization,” “feature extraction” and grammatical variants thereof refers to methods used to compute structural features of a molecule, for example a reference molecule (e.g., a reference protein) of the present disclosure. The structural features of the molecule can include, for example, angles, orientations, distances, interactions, variances, fluctuations, energies, solvent accessibility or lack thereof, or any combination therein for the present disclosure. Structural features can be defined by individual atoms or groupings of atoms according to structural elements (e.g. backbone, side chains, residues, motifs, secondary structural elements, or domains). In some aspects, molecular featurization results in at least a 10-fold, 20-fold, 30-fold, 40-fold, or 50-fold reduction in the number of features that define a molecular structure (e.g., a reference molecule or a variant molecule of the present disclosure). The featurization process does not require a human operator, i.e., the process is automated, and the particular featurization is chosen based on model performance downstream.

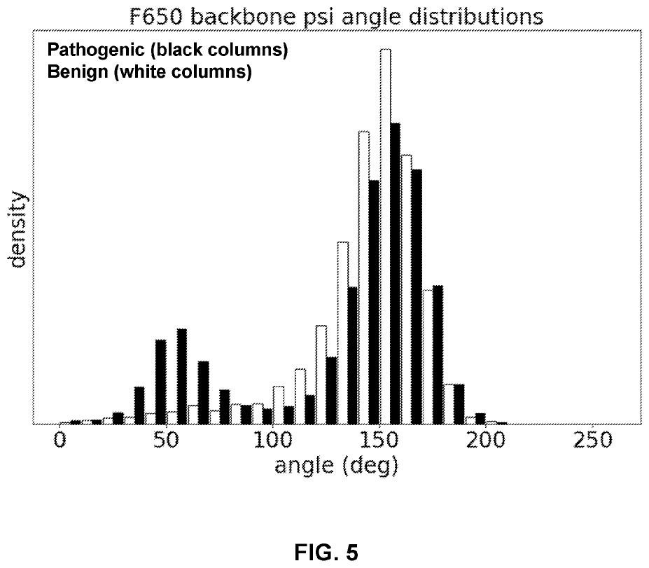

An example of the rationale behind the selection of a specific feature, e.g., backbone psi-angles, for dimensionality reduction is illustrated in FIG. 5, which shows that there is different distribution of backbone psi-angles at a particular position (F650) in pathogenic and benign variants of a molecule (e.g., variants comprising mutation at positions P349L, L341P, etc.). Thus, there is a relationship between a feature (backbone psi-angle distribution) and the pathogenicity of a variant molecule. Accordingly, backbone psi-angles are structural features that may be extracted for dimensionality reduction. The methods disclosed herein, also comprise a dimensionality reduction step. This dimensionality reduction step also does not require a human operator. Therefore, the dimensionality reduction step is unsupervised. Dimensionality reduction can be used to further reduce the number of overall structural features extracted from the conformational dynamics of a reference molecule (e.g., a reference protein) or a variant molecule (e.g., a variant protein) to generate a low-dimensional representation of the molecule. As used herein, the term “dimensionality reduction” includes feature selection (e.g., selecting features based on feature correlation, variance, dispersion or any combination thereof), linear decomposition (e.g., principal component analysis, PCA, and/or singular value decomposition, SVD), non-linear decomposition (e.g., manifold learning (e.g. UMAP), isomap, kernel PCA, t-distributed Stochastic Neighbor Embedding (t-SNE), and multidimensional scaling (MDS)), neural-network embedding (e.g., using an artificial neural network (ANN) to generate a low-dimensional representation of the input data, such as through the use of (variational) autoencoders or graphical embedding), or any combination thereof. Unsupervised dimensionality reduction can result in a further reduction in features by at least a 50-fold, 60-fold, 70-fold, 80-fold, 90-fold or 100-fold.

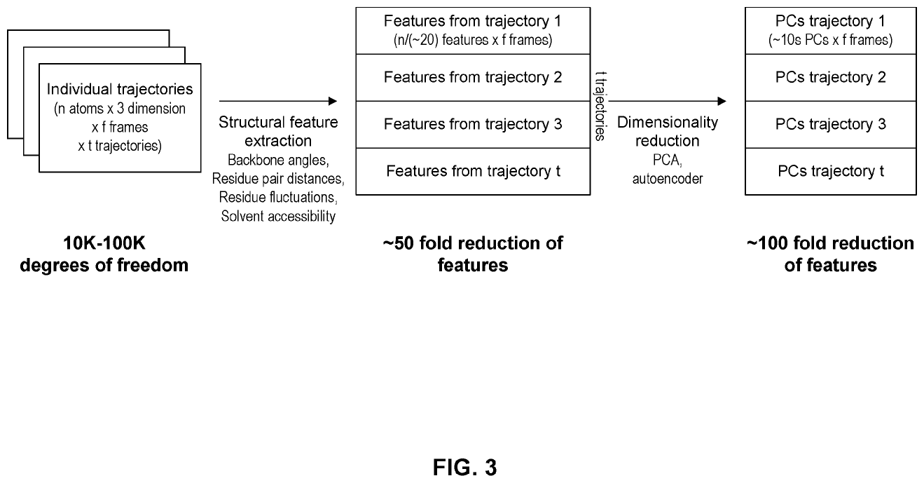

A schematic view of the dimensionality reduction portion of a workflow of the present disclosure, including (i) a structural feature extraction step, in which specific original features or characteristics are selected, and (ii) a dimensional reduction step, in which the selected original features or characteristics are converted into new features or characteristics using methods such as PCA or autoencoder, is presented in FIG. 3. As a result, large reductions in the dimensionality of the conformational dynamics can be achieved. For example, a system comprising multiple trajectories and having between 10,000 and 100,000 degrees of freedom can be subjected to a structure feature extraction step as disclosed herein (e.g., extracting features such as backbone angles, residue pair, distances, residue fluctuation, or solvent accessibility to name a few) to achieve, e.g., an approximately 50-fold reduction in number of features. After undergoing structural feature extraction, the trajectories can be subjected to a dimensionality reduction step as disclosed herein (e.g., using PCA, autoencoder, etc.) to yield a set of trajectories in which the number of features has been further reduced, e.g., approximately 100-fold.

The methods disclosed herein further comprise determining a metastable conformational state or set thereof from the low-dimensional conformational landscape of the reference molecule (e.g., a reference protein). The process of determining such metastable conformation state generally comprises (1) using clustering techniques to define a set of conformational clusters in the low-dimensional conformational landscape of the reference molecule (e.g., a protein), e.g., (a) defining a set of conformational states in the low-dimensional space by projecting the conformational states from the conformational dynamics of the reference protein structure onto the low-dimensional space, and (b) using the projected conformational states as inputs to clustering algorithms to define conformational clusters, (2) selecting a subset of conformational clusters from the set of all clusters, and (3) defining a representative conformational state from each selected conformational cluster as a metastable conformational state for further analysis. A “conformational cluster” is a collection of conformational states that are near each other in the low-dimensional conformational landscape, e.g., a region in the low-dimensional conformational landscape.

The disclosed methods can also comprise selecting or prioritizing a subset of the conformational clusters based on cluster properties, e.g., the distance from conformational landscape centroid, cluster occupancy, cluster distribution statistics, structural properties of the cluster, thermodynamics of the cluster, or any combination thereof. The structural properties can be selected from the structural featurizations used for dimensionality reduction for any number of structural components of the molecular system.

In some embodiments, the term “metastable conformational state” refers to a conformational state sampled in the conformational cluster that is closest or among the closest to the centroid of all conformational states of the cluster in the low-dimensional representation. In the methods disclosed herein, a metastable conformational state can be identified for each selected conformational cluster. In some embodiments, a metastable conformational state can also be defined as (i) a conformational state in the region of the conformational cluster generated by a generative model such as the decoder network of an autoencoder, or a state that is imputed from nearby conformational states in the conformational cluster, (ii) a local minimum in the conformational landscape, or (iii) a conformational state in which the molecular component of interest is distinct from other metastable conformational states by at least about 2 Å backbone atom root mean square deviation (RMSD).

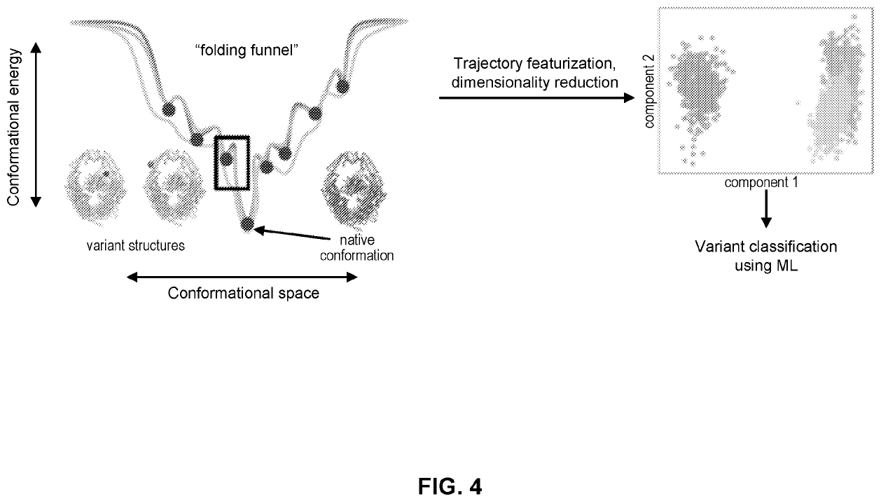

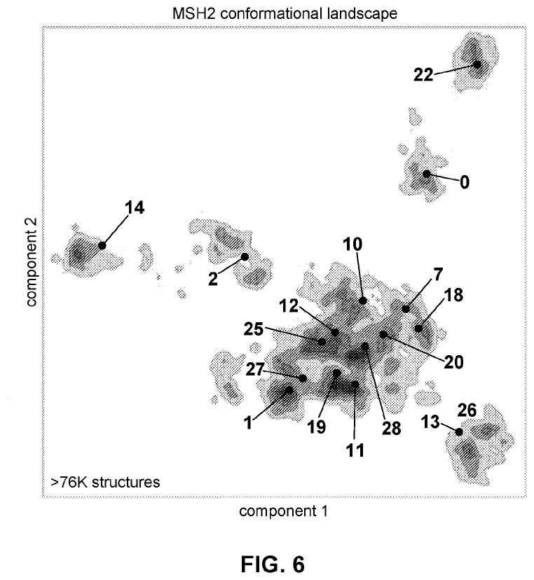

A schematic representation of the energetic state of the conformation of a reference molecule and the energetic states of metastable conformations of variant molecules is shown in FIG. 4. This representation exemplifies, as discussed above, that through trajectory featurization (reduction of degrees of freedom in the conformational dynamics by selecting specific features) and dimensionality reduction (conversion of selected features into other features for example via PCA) it is possible to simplify or compress the conformational space to generate simplified conformational dynamics while still distinguishing between reference molecule and variant molecules. In turn, as it will be discussed below, the simplified representation can be used as input to generate a machine-learning model. An exemplary low-dimensional conformation landscape representation resulting from clustering structural conformation of the MutS Homolog 2 protein (MSH2), which is associated with diseases such as Lynch Syndrome I or Mismatch Repair Cancer Syndrome 2, is shown in FIG. 6. Each one of the clusters presented in FIG. 6 represents a metastable conformation state.

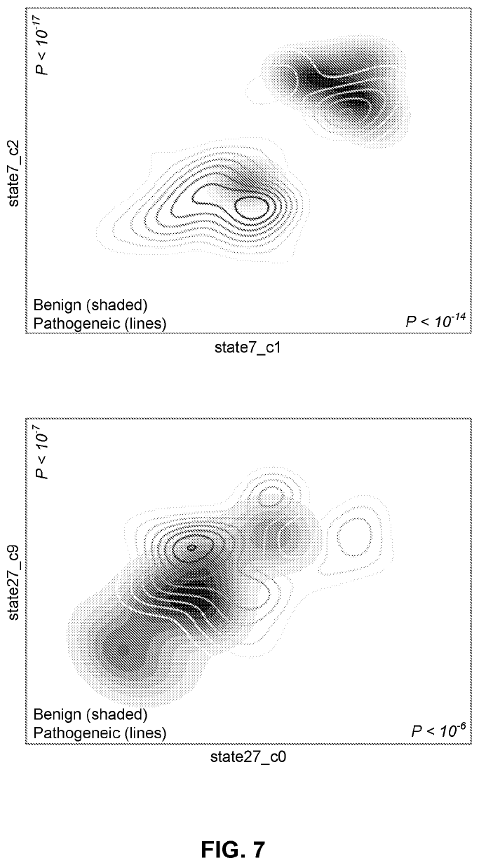

The conformational dynamics in a low-dimensional representation can distinguish between pathogenic and benign variants (e.g., missense gene variants), as shown, for example, in FIG. 7, and therefore they can be effectively used as features in a predictive machine learning model. Constructing a variant molecule (e.g., a variant protein) or set thereof can comprise introducing at least one variant (e.g., a mutation) to the reference molecule (e.g., a reference protein) in a metastable conformational state or set thereof. This process can comprise, e.g., (1) defining a variant or set thereof (e.g., a mutation or set of mutations) for which to construct variant molecules, e.g., a set of variants that are observed or could be observed in medical genetic testing, (2) introducing a variant (e.g., a mutation) into the relevant molecular component of the molecular system (as disclosed above, a molecular system can comprise, e.g., a reference molecule such as protein, and additional components such as solvents or ligands), (3) performing steric-based and/or energy-based minimization of the variant (e.g., a mutation in a reference molecule, which after the introduction of the variant would be considered a variant molecule) to mitigate abrupt conformational changes in the molecular system.

The set of variants can include (i) those with known clinical significance, to be used as training labels for a predictive model and (ii) a set of variants with unknown clinical significance but for which further variant effect information is desired. The set of variants should include variants that are known to change the composition of one or more molecular components of the system (e.g. missense variants that are known to change the amino acid residue at a particular position in a protein). The set of variants should include at least one variant known to cause a pathogenic effect on the molecular system and one variant known to cause a non-pathogenic (benign) effect on the molecular system (which may itself be the reference molecular system).

A variant (e.g., a mutation) can modify, e.g., the identity, properties, interactions or positions of atoms in a molecular structure. A variant can be, e.g., the mutation of an amino acid side chain or a nucleic acid base. A variant can be directly encoded by a variant gene (e.g. a substitution, insertion, deletion, missense mutation or nonsense mutation), or can occur indirectly, e.g., changes in the composition of the molecular system (e.g. the increase or decrease in the concentration of molecular components or a difference in the chemical composition of a molecular component), or a non-genetic modification of a molecular component (e.g. post-translational modifications such as phosphorylation or glycosylation).

In some aspects, introducing a variant (e.g., a missense variant) in a reference molecule structure to generate a variant molecule structure can comprise some or all of the following steps: (1) removing the atoms and interactions of a particular amino acid side chain at the variant position from the reference molecule (e.g., a reference protein), (2) determining the backbone phi-psi angles original position, (3) iteratively identifying likely rotamer conformations for the missense amino acid using a backbone-dependent rotamer library and calculating the steric clash caused by the placement of this rotamer at the variant position and repeating until the steric clash falls below a threshold, and (4) completing relaxations to further decrease the steric and energetic costs associated with introducing the variant.

Determining the conformational dynamics of the variant molecule structure in the metastable conformational state can comprise using computational methods to sample the conformational changes that occur after the introduction of the variant. These conformational changes can be determined using, e.g., molecular dynamics (MD) simulations.

The predictive models of the present disclosure can be generated, e.g., using machine-learning (ML) methods such as linear or non-linear regression, decision trees (e.g. a random forest), or an Artificial Neural Network (ANN). Clinical data can be used as training labels, e.g., biomarker status (e.g., presence or absence of a certain biomarker or its expression level), biometric data, lifestyle-related data, response to treatments, symptoms of the disease or conditions, protein expression data, type of treatment administered, dosage, dosage regimen, administration route, presence or absence of co-therapies, response to the therapy, age, body weight, gender, ethnicity, NCBI ClinVar database submissions, INVITAE™ clinical interpretations, data from other experimental or computational models, or any combination thereof. For example, an ANN may be trained with a training set comprising the variant protein structures obtained after determining conformational dynamics of metastable conformational states of the variant molecule. The training set may comprise structural changes caused by the introduction of the variant, and labels (training labels) applied to each variant protein structure corresponding, e.g., to observed clinical characteristics (e.g., likelihood or severity of a disease when a particular variant is present).

The prediction of variant effects can comprise training the predictive model using the conformational dynamic changes in the metastable conformational states of variant molecules and on training labels such as those described above applied to such variant molecules, and using the trained model to predict the clinical significance of other variants with unknown significance.

The predictive model can be extended to newly observed variants from genetic testing in a process comprising, e.g., (1) constructing new variant molecules (e.g., variant proteins) from the stored metastable conformational states, (2) generating conformational dynamics of the new variant molecules, (3) using previously computed unsupervised dimensionality reduction models to determine low-dimensional conformational dynamics for the new variant molecules, and (4) using a previously trained predictive model to predict the clinical significance of the newly observed variants.

### I. High-throughput Prediction of Variant Effects from Conformational Dynamics

The present disclosure describes novel computational methods that enable the characterization of the functional consequences, i.e., biological effects, of variants (e.g., missense gene and protein variants). These methods integrate conformational dynamics generation and machine learning (ML) to identify and quantify structural features of a molecular system (e.g., a gene or protein) and variants thereof (e.g., missense variants), and to obtain a measure or prediction of the effect of variants (e.g., mutations) and their potential disruption of biological function. In turn, the identification of functional disruptions, i.e., biological effects, with respect to a reference molecular system (e.g., a wild type protein or gene, or prevalent allelic form) and their severity can be used, e.g., to treat diseases and disorders associated with such functional disruptions.

In some aspects, the present disclosure provides computer implemented methods for high throughput prediction of biological effects of variants (e.g., missense variants) comprising
(a) generating conformational dynamics (e.g., from molecular dynamics) structures of a reference molecule (e.g., a wild type protein or gene, or prevalent allelic form);
(b) using unsupervised dimensionality reduction methods to define a low-dimensional conformational landscape of a reference molecular system comprising a reference molecule (e.g., a reference protein);
(c) determining a metastable conformational state or set thereof from the low-dimensional conformational landscape of the reference molecular system comprising the reference molecule (e.g., a reference protein);
(d) constructing a variant molecule or set thereof by introducing at least one variant (e.g. a mutation) in silico to a metastable conformational state or set thereof of molecular component in the reference molecular system, e.g., the reference molecule (e.g., a reference protein), thereby generating a variant molecular system comprising, e.g., a variant molecule (e.g., a variant protein) or a set thereof,
(e) determining the conformational dynamics of the variant molecular system or set thereof in the metastable conformational state or set thereof,
(f) using unsupervised dimensionality reduction methods to generate a low-dimensional representation of conformational dynamics of the variant molecular system or set thereof in the metastable conformational state or set thereof;
(g) training a predictive model, e.g., an ANN, using the low-dimensional representation of the conformational dynamics of the variant system or set thereof and biological or clinical data as training labels, wherein the predictive model predicts biological effects of variants on a molecular component (e.g., a protein) of the reference molecular system (e.g., a reference protein); and optionally,
(h) extending the predictive model to newly observed variants from medical genetic testing expeditiously, e.g., introducing a new variant for evaluation in step (d) of the workflow.

Since the different steps in the workflow would be pre-calculated after an initial iteration, the evaluation of additional variants would be highly expedited.

In general, the methods described herein include one or more of the steps described above. In some aspects, the order of the steps described above can be modified, e.g., the generation of conformational dynamics (e.g. using MD) of the variant molecule (e.g., a variant protein) or set thereof may be conducted prior to or simultaneously to simulating structural conformations of the reference molecule (e.g., a reference protein). In other aspects, specific groups of steps may be repeated in a loop. For example, the steps comprising generating a variant molecular system (e.g., molecular system comprising a variant protein structure) or set thereof by introducing at least one variant (e.g., a missense mutation) in silico in a metastable conformational state of the reference molecular system (e.g., molecular system comprising a reference molecule, for example a reference protein such as a wild type protein or a prevalent allelic form); and determining conformational dynamics (i.e., simulating structural conformations) of the variant system or set thereof, e.g., by applying molecular dynamics (MD); may be applied successively or concurrently to different batches or families of variants.

In some aspects, the methods described herein comprise: (a) generating conformational dynamics of a reference protein; (b) defining a low-dimensional conformational landscape of the reference protein structure using unsupervised dimensionality reduction methods; (c) determining a metastable conformational state or set thereof from the low-dimensional conformational landscape of the reference protein structure; (d) constructing a variant protein or set thereof by introducing at least one variant (e.g., mutation) in silico to a metastable conformational state or set thereof of the reference protein structure, thereby generating a variant protein structure or a set thereof, (e) determining the conformational dynamics of the variant protein structure or set thereof in the metastable conformational state or set thereof, (f) generating a low-dimensional representation of conformational dynamics of the variant protein or set thereof in the metastable conformational state or set thereof using unsupervised dimensionality reduction methods; (g) training a predictive model using the low-dimensional representation of the conformational dynamics of the variant protein or set thereof using biological or clinical data as training labels, wherein the model predicts biological effects of variants on the reference protein; and, optionally, (h) extending the predictive model to newly observed variants from medical genetic testing expeditiously. In some aspects, a new variant for evaluation can be introduced in the pipeline in step (d).

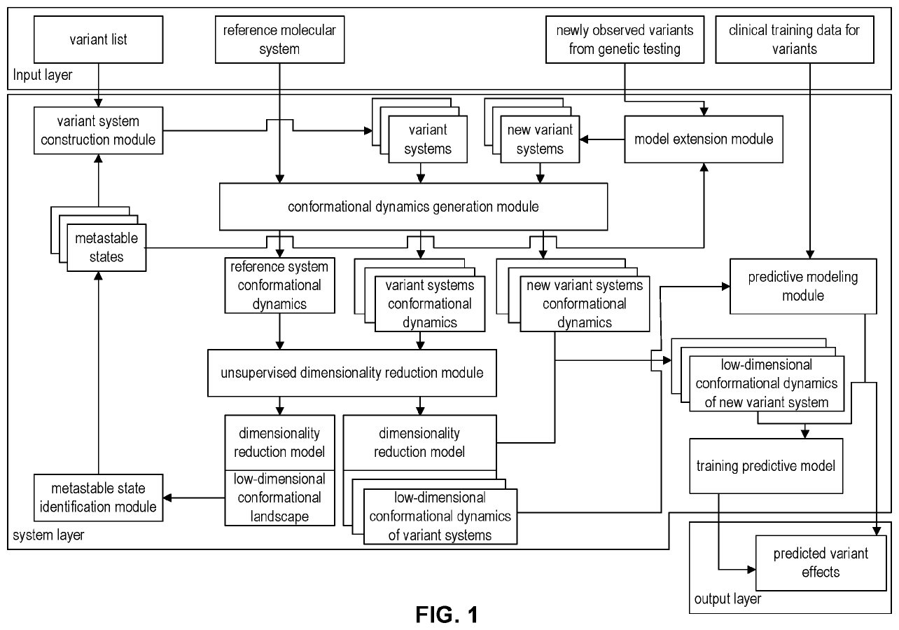

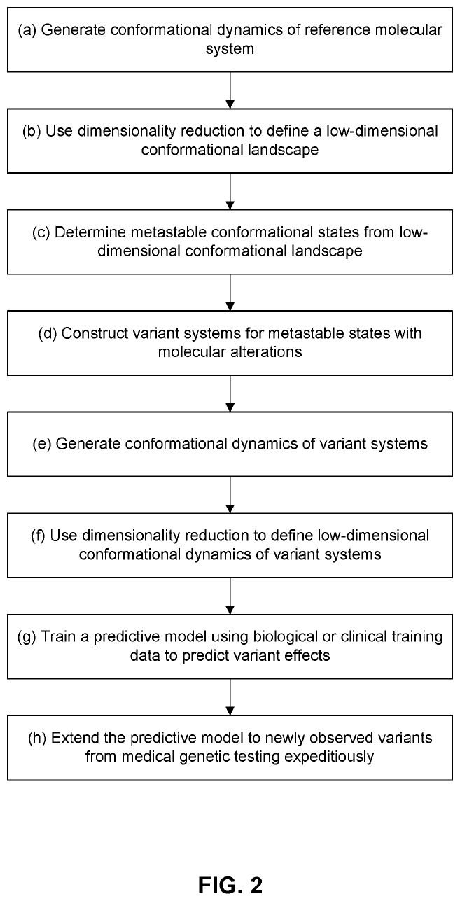

Schematic representations of exemplary workflows of the present disclosure are presented in FIG. 1 and FIG. 2. The diagram in FIG. 1 illustrates the main modules (conformational dynamics generation, unsupervised dimensionality reduction, metastable conformational state identification, variant construction, model extension, and predictive modeling; white background) and the data objects (reference molecule, reference molecule conformational dynamics, low-dimensional conformational landscape, metastable conformational states, variant list, variant molecules, variant molecule conformational dynamics, low-dimensional conformational dynamics of variant molecules, newly observed variants from genetic testing, clinical training data for variants, and predicted variant effects; gray background) that are inputs and outputs of the various modules.

The predictive system to predict variant effects from molecular conformational dynamic disclosed herein can comprise the following steps: (a) generating conformational dynamics of a reference molecule; (b) using unsupervised dimensionality reduction methods to define a low-dimensional conformational landscape of the reference molecule; (c) determining a metastable conformational state or set thereof from the low-dimensional conformational landscape of the reference molecule; (d) constructing a variant molecule or set thereof by introducing at least one molecular alteration to a molecular component in the reference molecule in a metastable conformational state or set thereof; (e) determining the conformational dynamics of the variant molecule or set thereof in the metastable conformational state or set thereof; (f) using unsupervised dimensionality reduction methods to generate a low-dimensional representation of conformational dynamics of the variant molecule or set thereof in the metastable conformational state or set thereof, (g) training a predictive model using the low-dimensional representation of the conformational dynamics of the variant molecule or set thereof using biological or clinical data as training labels, wherein the model predicts biological effects of variants on a molecular component of the reference molecule; and optionally (h) extending the predictive model to newly observed variants from medical genetic testing expeditiously. See FIG. 2.

### I.A Conformational Dynamics Generation

The methods disclosed herein use the conformational dynamics of molecular structures. Conformational dynamics can be generated using computer methods, e.g., molecular dynamics (MD) simulations, for studying the movements of atoms and molecules in a system comprising, e.g., a reference molecule or variant molecule of the present disclosure. Conformational dynamics represent the complex time-dependent behavior of atoms and molecules in a system that can comprise, e.g., large molecules with highly dynamic structures such as proteins and nucleic acids. Such dynamics include, for example, molecule stability, molecule folding, conformational changes of molecules, and molecular binding. Thus, in the case of protein, such dynamics comprise, for example, protein stability, protein folding, conformational changes of proteins, and molecular binding including receptor/ligand binding. These studies can provide, for example, useful information for designing drugs for the treatment of various diseases including cancer and genetic diseases. They also provide information for predicting the severity of a disruption, such as a variant (e.g., a mutation), to the biological function of a molecule, for example, a macromolecule such as a protein (e.g., a reference protein or a variant protein such as a missense variant protein of the present disclosure).

The study of the conformational dynamics of molecules such as proteins is complex and requires computers and in silico study of the macromolecular structure and macromolecular dynamics in order to obtain accurate information. In some aspects, the methods disclosed herein can use all-atom conformation dynamics simulations, e.g., MD simulations, conducted using any software, any parameter set or force field used to calculate the potential energy, and any protein/ligand interaction system. The parameter sets include rule sets that govern the behavior of individual atoms in the system. The molecular structures generated through conformation dynamics simulations, e.g., MD simulations, can be characterized by several metrics for comparing the impact of a variant (e.g., a mutation) on conformational dynamics of a references molecule or variant molecule of the present disclosure, for example: global structural features, dynamics of subdomains in the structure, energetic interactions, and statistical characteristics of overall collection of structures.

In addition to being computationally intensive, conformational dynamics (e.g., from MD simulations) are high dimensional as they contain the movement of all atoms within a molecule in three dimensions over time. There are often more than 10,000 atoms in molecules, so analysis of these high-dimensional systems typically requires expert analysis to determine features of interest. The need of using human experts to determine features of interest introduces a bottleneck that hinders the application of these methods in a high-throughput mode. Therefore, there is a need to develop methods that can extract relevant structural features and reduce the dimensionality of conformational dynamics data, e.g., MD data, in an automated fashion (i.e., without human intervention) in order to use conformational dynamics data, e.g., MD data, for high-throughput variant classification, e.g., using machine-learning.

In some aspects, simulating structural conformations of the reference molecule (e.g., a reference protein) or a variant molecule (e.g., a variant protein) using conformational dynamics comprises (i) retrieving three-dimensional structures of the reference molecule (e.g., a reference protein) or variant molecule (e.g., a variant protein); and, (ii) generating conformational dynamics (e.g., from MD) using the retrieved three-dimensional structures of the reference molecule (e.g., a reference protein) or variant molecule (e.g., a variant protein). In some aspects, the three-dimensional molecular structures are experimentally determined (e.g. by crystallography, electron microscopy or nuclear magnetic resonance). In some aspects, the three-dimensional molecular structures are retrieved from an experimental three-dimensional protein structure repository such as PDB.

Knowing a molecule's (e.g., a protein's) structure provides an important resource for understanding how it functions, but experiments to determine the molecule's structure can take months or longer, and some prove to be intractable. For this reason, researchers have been developing computational methods to predict molecular structures. For example, researchers have developed computational methods to predict protein structures from their primary amino acid sequences. In cases where the three-dimensional structure of a similar protein has already been experimentally determined, e.g., using X-ray crystallography, algorithms based on “template modelling” are able to provide accurate predictions of the protein structure. Accordingly, in some aspects, the three-dimensional structures are template-modeled structures.

In some aspects, the three-dimensional molecular structures correspond to molecules (e.g., proteins) for which experimentally determined molecular structures are not available. Thus, in some aspects, the three-dimensional molecular structures are obtained via computational modeling. In some aspects, the free-modelled three-dimensional molecular structures are retrieved from databases with computationally derived structures such as modbase, Pfam, EMDB, or EVFold. In some aspects, three-dimensional molecular structures are obtained through prediction using an existing computational structure prediction software such as AlphaFold or trRosetta. See, e.g., Senior et al. (2020) Nature 577: 706-710 and Yang et al. (2020) PNAS 117:1496-1503, both of which are herein incorporated by reference in their entirety. In some aspects, the three-dimensional molecular structures are determined through a combination of experimental and computational methods

In some aspects, the three-dimensional molecular structures used in the methods disclosed herein are selected according to their resolution. In some aspects, the three-dimensional molecular structures used in the methods disclosed herein are high-resolution molecular structures.

As used herein, the term “resolution” refers to a measure of the quality of the data that has been collected for experimentally-derived molecular structures (e.g., the structure of a reference molecule or variant molecule of the present disclosure). For example, for molecular structures obtained using X-ray crystallography, if all of the proteins in the diffracted crystal are aligned in an identical way, forming a very perfect crystal, then all of the proteins will scatter X-rays the same way, and the diffraction pattern will show the fine details of the crystalized protein. On the other hand, if the proteins in the crystal are all slightly different, due to local flexibility or motion, the diffraction pattern will not contain as much fine information. Thus, resolution is a measure of the level of detail present in the diffraction pattern and the level of detail that will be seen when the electron density map is calculated. “High-resolution,” as applied to crystallographic structures, refers to those structures with resolution values of 1 Å or so, which are highly ordered and it is easy to see every atom in the electron density map. “Low resolution,” as applied to crystallographic structures, refers to those structures with resolution of 3 Å or higher which show only the basic contours of the protein chain, and the atomic structure must be inferred. Most crystallographic-defined structures of proteins fall in between these two extremes. As a general rule of thumb, there is a higher confidence in the location of atoms in molecular structures with resolution values that are small, i.e., “high-resolution structures.”

In some aspects, the term “high-resolution” means, e.g., a resolution of 3.0 Å or smaller in experimentally derived structure, e.g., a crystal structure of a reference molecule (e.g., a reference protein). Molecular structures of any resolution, such as, 6.0 Å, 5.0 Å, 4.0 Å, 3.0 Å, 2.0 Å or smaller can be employed in the disclosed methods. Thus, in some aspects, the resolution is about 1 Å, about 1.5 Å, about 2 Å, about 2.5 Å, about 3 Å, about 3.5 Å, about 4 Å, about 4.5 Å, about 5 Å, about 5.5 Å, or about 6 Å. In some aspects, the resolution is better (higher) than about 6 Å, better than about 5 Å, better than about 4 Å, better than about 3 Å, better than about 2 Å, or better than about 1 Å. In some aspects, the resolution is between about 1 Å and about 2 Å, between about 2 Å and about 3 Å, between about 3 Å and about 4 Å, between about 4 Å and about 5 Å, between about 5 Å and about 6 Å, between about 1 Å and about 3 Å, between about 2 Å and about 4 Å, between about 3 Å and about 5 Å, between about 4 Å and about 6 Å, between about 1 Å and about 4 Å, between about 2 Å and about 5 Å, between about 3 Å and about 6 Å, between about 1 Å and about 5 Å, between about 2 Å and about 6 Å, or between about 1 Å and about 6 Å.

In some aspects, the three-dimensional structures of the molecules used in the methods disclosed herein are selected according to the energetics of the conformational dynamics, e.g., from MD simulations. While resolution is a consideration for experimentally derived structures, it does not apply to computationally predicted structures. In addition to using structure and prediction quality metrics (e.g. resolution) as criteria for selecting targets, conformational dynamics, e.g., from MD simulations, can be used to evaluate whether a reference molecule structure (e.g., the three-dimensional structure of a reference protein) is amenable to building a variant molecule structure model. Conformational dynamics (e.g., from MD simulations of protein structures) can be evaluated on changes in secondary, tertiary and, in the case of complexes, quaternary structure and/or changes in solvent-accessible surface area to determine if the conformational dynamics of the reference molecule (e.g., a reference protein) can be accurately computed.

Conformational dynamics of a reference molecule or variant molecule (e.g., a reference protein or variant protein) are determined by applying computer methods, e.g., MD simulations, to the three-dimensional structure of the reference molecule or variant molecule (e.g., a reference protein or variant protein). MD is a computer simulation method for analyzing the physical movements of atoms and molecules. The atoms and molecules are allowed to interact for a fixed period of time, giving a view of the dynamic “evolution” of the system. In the most common version, the trajectories of atoms and molecules are determined by numerically solving Newton's equations of motion for a system of interacting particles, where forces between the particles and their potential energies are often calculated using interatomic potentials or molecular mechanics force fields. Because molecular systems typically consist of a vast number of particles, it is impossible to determine the properties of such complex systems analytically; MD simulation circumvents this problem by using numerical methods.

Other methods of generating conformational dynamics besides MD can be used to determine the conformational landscape of a protein (www.sciencedirect.com/science/article/pii/S0969212619301145). The method relies not on simulating conformational dynamics, e.g., MD simulations per se, but having a collection of discrete conformational states that represent the conformational landscape. A method such as this, which incorporates Deep Learning to determine the conformational landscape of a molecule could be used in place of traditional MD. Normal mode analysis (pubs.acs.org/doi/10.1021/cr900095e) may also be used to calculate conformational states of a protein akin to traditional MD.

Simulation size, timestep, and total time duration must be selected so that the calculation can finish within a reasonable period of time. However, the conformational simulations should be long enough to be relevant to the time scales of the natural processes being studied. To make statistically valid conclusions from the conformational simulations, the time span simulated should match the kinetics of the natural process. For example, the dynamics of proteins use data from simulations spanning nanoseconds (10⁻⁹ s) to microseconds (10⁻⁶ s). To obtain these conformational simulations, several CPU-days to CPU-years are needed. Parallel algorithms allow the load to be distributed among CPUs; an example is the spatial or force decomposition algorithm. During a classical MD simulation, the most CPU intensive task is the evaluation of the potential as a function of the particles' internal coordinates. Another factor that affects total CPU time needed by a simulation is the size of the integration timestep. This is the time length between evaluations of the potential. The timestep must be chosen small enough to avoid discretization errors (i.e., smaller than the period related to the fastest vibrational frequency in the system). Typical timesteps for classical MD are in the order of 1 femtosecond (10⁻¹⁵ s).

Given the CPU time requirements for generating the conformational dynamics of a single protein, MD-based methods are ill suited for high-throughput analyses. However, as disclosed in detail below, the currently disclosed methods are able to reduce the dimensionality of the data used in MD simulations, drastically reduce CPU time, and therefore make possible the application of conformational dynamics, e.g., from MD simulations, in high throughput mode.

In some aspects, conformational dynamics, e.g., from MD simulations, are generated using the GROMACS package/program. In some aspects, conformational dynamics, e.g., from MD simulations, are generated using the NAMD package/program. In some aspects, conformational dynamics, e.g., MD simulations, are generated using the OpenMM package/program. In some aspects, conformational dynamics, e.g., from MD simulations, are generated using the AMBER package/program. In some aspects, conformational dynamics, e.g., from MD simulations, are generated using the LAMMPS package/program. In some aspects, conformational dynamics, e.g., from MD simulations, are generated using the CHARMM package/program. In some aspects, conformational dynamics, e.g., from MD simulations, are generated using the Abalone package/program. In some aspects, conformational dynamics, e.g., from MD simulations, are generated using the TINKER package/program.

In some aspects, conformational dynamics, e.g., from MD simulations, are generated using the GROMOS force field. In some aspects, conformational dynamics, e.g., from MD simulations, are generated using the OPLS (Optimized Potentials for Liquid Simulations) force field. In some aspects, conformational dynamics, e.g., from MD simulations, are generated using an AMBER (Assisted Model Building with Energy Refinement) force field. In some aspects, the AMBER force field is ff14SB. In some aspects, conformational dynamics, e.g., from MD simulations, are conducted using a CHARMM (Chemistry at Harvard Macromolecular Mechanics) force field. In some aspects, the CHARMM force field is CHARMM19 (united-atom, sometimes termed extended atom), CHARMM22 (all-atom), CHARMM22/CMAP (dihedral potential corrected variant of CHARMM22), CHARMM27, CHARMM36, CHARMM36m or CHARMM36IDPSFF. In some aspects, conformational dynamics, e.g., from MD simulations, are conducted using a MMFF (Merck molecular force field) force field.

In some aspects, the reference molecule or variant molecule (e.g., a reference protein or variant protein) can be coarse-grained, such as using the MARTINI force field to reduce the number of atoms (and therefore calculations and time) needed to simulate the reference molecule or variant molecule (e.g., a reference protein or variant protein). By coarse-graining the reference molecule or variant molecule (e.g., a reference protein or variant protein), the simulation time of the reference molecule or variant molecule (e.g., a reference protein or variant protein) can be reduced.

In some aspects, solvent molecules are needed to simulate the reference molecule or variant molecule (e.g., a reference protein or variant protein) in a solvent environment. In some aspects, these solvent molecules are explicit water molecules (such as TIP3P). In some aspects, these solvent molecules include ion molecules (such as Na⁺ or Cl⁻). In some aspects these solvent molecules are lipids, either all-atom or coarse-grain atoms. In some aspects these solvent molecules can be represented with an implicit solvent model to reduce the number of atoms in the simulation.

In one specific aspect, the CHARMM36 force field with explicit TIP3P solvent is used. Minimization simulation of target system is done with over 5,000 simulation steps with constrained backbone atoms, followed by equilibration over 125 picoseconds up to a temperature of 303 K, followed by production simulations that were sampled ever 2 femtoseconds using a 1.2 nm radius Particle-Mesh Ewald electrostatic range and 1.2 nm radius cut-off for Van der Waals interactions.

While parallelizing simulations, reducing their complexity using coarse-graining or implicit solvent, and improving sampling using advanced sampling methods reduces the amount of simulation time required, simulating each variant molecule with the same amount of sampling as the reference protein would still be prohibitively compute, time, and cost intensive to be used in a high-throughput clinical variant interpretation platform. However, as described herein, by using the reference molecule conformational simulation to generate a low-dimensional representation of the conformational landscape, and clustering the conformations in the landscape to identify metastable conformational states from which to simulate variant molecules, the total amount of simulation time for the variants can be reduced. In one specific aspect, a reference protein was simulated for approximately 5 microseconds of simulation time, totaling more than 300 hours of compute time. In comparison, for each variant protein simulation, only 100 nanoseconds (7 hours of compute time) simulation time was required to generate a performant model, i.e., selecting a pre-computed metastable conformational state identified as described herein, reduced total computation time to about 2% of the original time. In other words, in the time that a conventional conformation dynamics approach would calculate a conformational simulation for a single variant protein, the present methods may conduct conformational dynamics simulations for over 40 variant proteins. It is important to emphasize that these reductions in simulation time are a result of using a low-dimensional representation of the conformational landscape, and of clustering the conformations in the landscape to identify metastable conformational states, and are applicable to any simulation approach independently of the simulation package/program and/or advanced sampling method used.

In some aspects, each one of the generation of conformational dynamics steps (e.g., via MD simulation) in the methods disclosed herein comprises generating independently at least about 1,000, at least about 5,000, at least about 10,000, at least about 20,000, at least about 30,000, at least about 40,000, at least about 50,000, at least about 60,000, at least about 70,000, at least about 80,000, at least about 90,000, at least about 100,000, at least about 200,000, at least about 300,000, at least about 400,000, at least about 500,000, at least about 600,000, at least about 700,000, at least about 800,000, at least about 900,000, or at least about 1,000,000 simulated structural conformations for each molecule (e.g., a reference molecule such as a reference protein, or any variant thereof). In some aspects, each one of the generation of conformational dynamics steps (e.g., via MD simulation) in the methods disclosed herein comprises generating independently about 1,000, about 5,000, about 10,000, about 20,000, about 30,000, about 40,000, about 50,000, about 60,000, about 70,000, about 80,000, about 90,000, about 100,000, about 200,000, about 300,000, about 400,000, about 500,000, about 600,000, about 700,000, about 800,000, about 900,000, or about 1,000,000 simulated structural conformations for each molecule (e.g., a reference molecule such as a reference protein, or any variant thereof). In some aspects, each one of the generation of conformational dynamics steps (e.g., via MD simulation) in the methods disclosed herein comprises generating independently about 1,000 to about 50,000, about 50,000 to about 100,000, about 100,000 to about 150,000, about 150,000 to about 200,000, about 200,000 to about 250,000, about 250,000 to about 300,000, about 300,000 to about 350,000, about 350,000 to about 400,000, about 450,000 to about 500,000, about 500,000 to about 600,000, about 600,000 to about 700,000, about 700,000 to about 800,000, about 800,000 to about 900,000, or about 900,000 to about 1,000,000 simulated structural conformations for each molecule (e.g., a reference molecule such as a reference protein, or any variant thereof). In some aspects, each one of the generation of conformational dynamics steps (e.g., via MD simulation) in the methods disclosed herein comprises generating independently at least 1,000,000 simulated structural conformations for each molecule (e.g., a reference molecule such as a reference protein, or any variant thereof). In some aspects, the term “simulation” refers to all the simulated structural conformations for a given protein. The terms “conformational dynamics,” “simulated structural conformation,” “conformational simulation,” and grammatical variants thereof are used interchangeably and refer to the three-dimensional conformations of a reference molecule (e.g., a reference protein) or variant molecule (e.g., a variant protein) obtained via the application of methods that generate conformational dynamics, e.g., molecular dynamics (MD).

In some aspects, each one of the generation of conformational dynamics steps (e.g., via MD simulation) steps in the methods disclosed herein, e.g., steps (a) and (e) of the workflow described above, comprises independently generating about 1,000, about 5,000, about 10,000, about 20,000, about 30,000, about 40,000, about 50,000, about 60,000, about 70,000, about 80,000, about 90,000, about 100,000, about 150,000, about 200,000, about 250,000, about 300,000, about 350,000, about 400,000, about 450,000, about 500,000, about 600,000, about 700,000, about 800,000, about 900,000, or about 1,000,000 simulated structural conformations. In some aspects, each one of generation of conformational dynamics steps (e.g., via MD simulation) steps in the methods disclosed herein, e.g., steps (a) and (e) of the workflow described above, comprises generating independently between about 1,000 and about 5,000, between about 5,000 and about 10,000, between about 10,000 and about 20,000, between about 20,000 and about 50,000, between about 50,000 and about 100,000, between about 100,000 and about 200,000, between about 200,000 and about 300,000, between about 300,000 and about 400,000, between about 400,000 and about 500,000, between about 500,000 and about 600,000, between about 600,000 and about 700,000, between about 700,000 and about 800,000, between about 800,000 and about 900,000, between about 900,000 and about 1,000,000, between 10,000 and about 50,000, between about 10,000 and about 100,000, between about 25,000 and about 100,000, between about 10,000 and about 150,000, between about 10,000 and about 200,000, or between 50,000 and about 150,000 simulated structural conformations. Because generating conformational dynamics is time- and cost-intensive, using advanced sampling that have been developed by the MD community can increase the effective amount of simulation time with the same amount of computing time/computing cost. Accordingly, in some aspects, generating conformational dynamics disclosed herein comprise using enhanced sampling MD methods. Most of the enhanced sampling methods fall into two categories: collective variable based and collective variable free methods. The bottleneck of generating conformational dynamics lies in the fact that there are high-energy barriers separating different structures/conformations so that transitions between them appear as rare events. A direct and effective idea to accelerate the thermodynamics calculation is to modify the potential energy surface by adding bias potential to the Hamiltonian of the systems, i.e., decreasing the energy barrier to increase the sampling transition regions. Such methods include but are not limited to the widely used umbrella sampling, potential smoothing methods, J-walking, local elevation, conformational flooding, hyperdynamics, conformational space annealing, adaptive biasing force method, metadynamics (MetaD), local elevation umbrella sampling, and variationally enhanced sampling (VES). In some aspects, to guide effectively the simulations, these methods use predefined reaction coordinates or collective variables (CVs). However, it is well known that the proper reaction coordinates are not easily identified for many systems. Under such a situation, the collective variable free method can be helpful. In this latter kind of method, the canonical probability distribution is altered to a distribution that will induce a broader sampling of the potential energy. A number of such methods have been proposed, e.g., parallel tempering or replica exchange molecular dynamics (REMD), simulated tempering, multicanonical simulation, temperature-accelerated dynamics, Wang-Landau algorithm, accelerated molecular dynamics simulation, statistical temperature sampling, temperature-accelerated molecular dynamics, enveloping distribution sampling, integrated tempering sampling (ITS), and accelerated enveloping distribution sampling. In some aspects, the enhanced sampling method is MetaD, VES, REMD, or ITS. In some aspects, the enhanced sampling method is a hybrid method combining the features of different enhanced sampling methods. See Yi et al (2019) J. Chem. Phys. 151:070902, which is herein incorporated by reference in its entirety.

### I.B Unsupervised Dimensionality Reduction

In some aspects, the methods disclosed herein comprise processing steps in which the dimensionality of conformational dynamics (e.g., from MD simulations) is reduced. For example, the steps comprising processing the conformational dynamics of the reference molecule (e.g., reference protein) or corresponding variant molecule (e.g., a missense variant protein) to generate a low-dimensional representation of the conformational landscape of the reference molecule or variant molecule, comprise independently processing of the conformational dynamics of the molecule or the variant molecule (or a set of variant molecules) to generate low-dimensional representations of the respective conformational landscapes of the reference molecule (e.g., a reference protein) and each variant thereof (e.g., variant proteins).

As used herein, the term “conformational landscape” refers to the set of conformations (e.g., structural conformations) that can be adopted by a reference molecule (e.g., reference protein) or corresponding variant molecule (e.g., a missense variant protein) of the present disclosure. The large number of atoms in a molecular system creates a high dimension dataset.

As used herein the term “dimensionality,” as applied to conformational dynamics (e.g., MD) of the present disclosure, refers to the number of dimensions or degrees of freedom present in a set of conformational dynamics i.e., simulated structural conformations, generated according to the methods disclosed herein.

High dimensional data is often sparse, which presents a problem for any method that requires statistical significance. In machine learning problems that involve learning a “state of nature” from a finite number of data samples in a high-dimensional feature space with each feature having a range of possible values, typically an enormous amount of training data is required to ensure that there are several samples with each combination of values.

The conformational dynamics (e.g., from MD) for a reference molecule (e.g., reference protein) or corresponding variant molecule (e.g., a missense variant protein) of the present disclosure can comprise, e.g., between 10,000 and 100,000 degrees of freedom (n non-solvent atoms×3 dimensions×f frames×t trajectories). The high dimensionality of the conformational dynamics (e.g., from MD) precludes the meaningful application of machine learning methods, and consequently prevents scaling up the system to a true high-throughput mode. Thus, lower dimensional representations of conformational space are required. These low dimensionality representations of conformational space are sufficiently simplified to be used to train a machine learning system.

The process to generate a low dimensionality representation of conformational space according to the present disclosure comprises two defined stages: structural feature extraction and dimensionality reduction. Structural feature extraction returns a subset of the features (e.g., backbone angles), whereas dimensionality reduction creates new features from functions of the original features, e.g., a subset of extracted features.

In some aspects, processing the conformational dynamics (e.g., from MD) to generate a low-dimensional representation of the conformational landscape of a reference molecule (e.g., reference protein) or corresponding variant molecule (e.g., a missense variant protein) comprises a feature selection process in which structural features are selected and computed from the conformational dynamics (e.g., from MD) as a first dimensionality reduction step. This feature selection approach tries to find a subset of input variables (also called features or attributes).

As used herein, the term “structural feature” refers to features or attributes in theconformational dynamics (e.g., from MD) for a reference molecule (e.g., reference protein) or corresponding variant molecule (e.g., a missense variant protein) that define or affect a particular conformational state of the reference or variant molecule. The structural features are not limited to purely structural characteristics of a molecule (e.g., a protein), but can also include features or attribution related to molecular function (e.g., protein function) or thermodynamics. In some aspects, the structural features relate to molecular geometry (e.g., protein geometry). In some aspects, the structural features related to protein geometry comprise, e.g., atom positions, bond angles (e.g., backbone bond angles), backbone bond lengths, inter-residue distances and/or orientations, stereo chemical conformation, secondary structure, tertiary structure, etc. In some aspects, the structural features relate to backbone bond angles. In some aspects, the structural features related to backbone bond angles are phi (<p) angles, psi (y) angles, omega (o) angles, or any combination thereof. In some aspects, the structural features comprise side-chain chi angles (x). In some aspects, the structural features relate to protein physicochemical properties. In some aspects, the structural features related to protein physicochemical properties comprise, e.g., surface accessibility, surface energy, solvent accessibility, etc. In some aspects, the structural features relate to domain architecture, e.g., domain relative orientations, or subdomains, for example, a protruding loop or strand, which may adopt different local dynamics relative to the motion of the overall structure. Thus, dynamics of subdomains in structure can be obtained.

In some aspects, the structural features refer to binding properties such a protein/ligand interaction, e.g., protein-protein interaction, protein-ion interaction, protein-small molecule interactions, or protein-nucleic acid interactions. In some aspects, the structural features refer, for example, to the geometry, affinity, or specificity of a cofactor-binding site or a substrate-binding site. In some aspects, the ligand can be a substrate, when the target protein and corresponding missense variant protein are an enzyme. In some aspects, the ligand can also be an agonist, an antagonist, an effector molecule, or an antibody. Energetic interactions that refer to the sum of all atomic interactions between the protein and ligand can be obtained.

In some aspects, the structural features refer to root-mean-squared fluctuations (RMSFs) and/or root-mean-square deviations (RMSDs).

In some aspects, the structural features used to reduce to total number of features in the conformational dynamics (e.g., from MD) for a reference molecule (e.g., reference protein) or corresponding variant molecule (e.g., a missense variant protein) comprise phi (p) angles, psi (y) angles, omega (o) angles, residue pair distances (inter-residue distances), residue fluctuations, solvent accessibility, distance from the center of mass to a binding site or to a ligand bound to such binding site, global structural features related to protein shape (e.g., length along different axes), domain relative orientations, binding-site interactions, energetic features such as van der Waal forces, electrostatic forces, and non-bonded energies occurring, for example, between a ligand and a protein, and any combinations thereof.

In some aspects, the extraction of structural features from the conformational dynamics (e.g., from MD) for a reference molecule (e.g., reference protein) or corresponding variant molecule (e.g., a missense variant protein) of the present disclosure can result in at least an about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, about 60-fold, about 70-fold, about 80-fold, about 90-fold, or about 100-fold reduction in structural features with respect to the total number of features present in the conformational dynamics (e.g., from MD) prior to the extraction of structural features. In some aspects, the extraction of structural features from the conformational dynamics (e.g., from MD) for a reference molecule (e.g., a reference protein) or corresponding variant molecule (e.g., a missense variant protein) of the present disclosure can result in at least an about 2-fold to about 5-fold, about 5-fold to about 10-fold, about 10-fold to about 20-fold, about 20-fold to about 30-fold, about 30-fold to about 40-fold, about 40-fold to about 50-fold, about 50-fold to about 60-fold, about 60-fold to about 70-fold, about 70-fold to about 80-fold, about 80-fold to about 90-fold, or about 90-fold to 100-fold reduction in structural features with respect to the total number of features present in the conformational dynamics (e.g., from MD) prior to the extraction of structural features.

Processing of the conformational dynamics can further comprise a dimensionality reduction step. Thus, in some aspects, the extraction of structural features from the conformational dynamics (e.g., from MD) can be followed by a step of dimensionality reduction conducted using, e.g., principal component analysis (PCA) or autoencoders, in which the total number of features present in the simplified conformational states resulting from structural feature extraction steps can be further reduced (or transformed into new features). Methods such as PCA or autoencoders transform the data from the high-dimensional space to a space of fewer dimensions. The data transformation can be linear, as in PCA, but many nonlinear dimensionality reduction techniques also exist.

Principal component analysis (PCA) is the process of computing the principal components of a collection of data points and using them to perform a change of basis on the data, sometimes using only the first few principal components and ignoring the rest. A variation of PCA, called time-structure Independent Component Analysis or tICA, is particularly useful for simulation as it identifies components that vary slowly along the time dimension, eliminating variation that is less likely to be useful for understanding protein conformational motions.

An autoencoder is a type of artificial neural network used to learn efficient data codings in an unsupervised manner. Examples are regularized autoencoders (Sparse, Denoising and Contractive), which are effective in learning representations for subsequent classification tasks, and Variational autoencoders, with applications as generative models.

For PCA and autoencoders, the number of output features is chosen in order to account for a significant portion of variation in the data.

In some aspects, dimensionality reduction of the conformational dynamics (e.g. from MD) can be conducted, e.g., using PCA, time-structure Independent Component Analysis (tICA), non-negative matrix factorization (NMF), Kernel PCA, Graph-based kernel PCA, linear discriminant analysis (LDA), generalized discriminant analysis (GDa), autoencoder, T-distributed stochastic neighbor embedding (t-SNE), or uniform manifold approximation and projection (UMAP).

In some aspects, structural feature extraction and dimensionality reduction can be combined into a single steps using, e.g., PCA, LDA, NMF, or canonical correlation analysis (CCA) techniques as a pre-processing step followed by K-NN on feature vectors in reduced-dimension space. In some aspects, the dimensionality reduction step results in at least an about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, about 60-fold, about 70-fold, about 80-fold, about 90-fold, or about 100-fold reduction in structural features with respect to the total number of features present in the conformational dynamics (e.g., from MD) prior to the extraction of structural features. In some aspects, the dimensionality reduction step results in at least a 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 60-fold, 70-fold, 80-fold, 90-fold, or 100-fold reduction in structural features with respect to the total number of features present in the conformational dynamics (e.g., from MD) after the extraction of structural features. In some aspects, the dimensionality reduction step results in at least about 3-fold to about 5-fold, about 5-fold to about 10-fold, about 10-fold to about 20-fold, about 20-fold to about 30-fold, about 30-fold to about 40-fold, about 40-fold to about 50-fold, about 50-fold to about 60-fold, about 60-fold to about 70-fold, about 70-fold to about 80-fold, about 80-fold to about 90-fold, or about 90-fold to 100-fold reduction in structural features with respect to the total number of features present in the conformational dynamics (e.g., from MD) after the extraction of structural features.

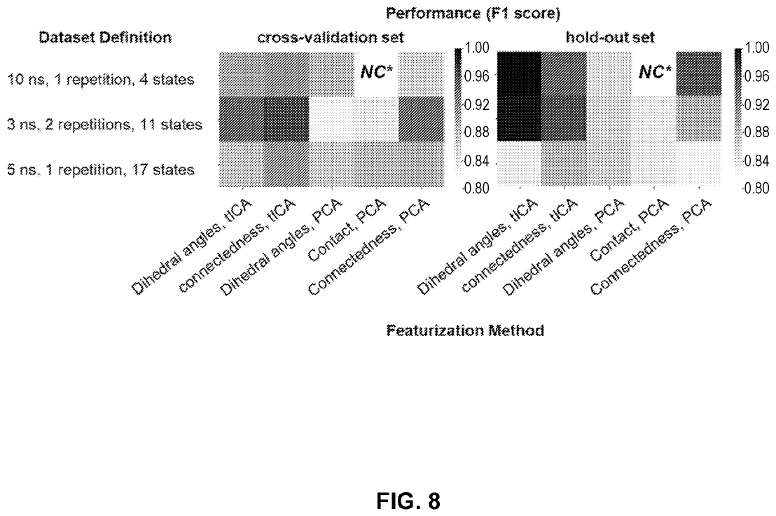

Specific featurization methods, designated according to the feature used followed by the dimensionality reduction method applied, e.g., dihedral angles, tlPCA; connectedness, tlCA; dihedral angles, PCA; contact, PCA; and connectedness, PCA are illustrated in FIG. 8.

### I.C Metastable Conformational State Identification

In some aspects, the methods disclosed herein comprise a processing step related to extracting a conformational state, for example, a metastable conformational state, from the low-dimensional conformational landscape of the reference molecule (e.g., a reference protein) to seed conformational dynamics generation (e.g. MD) of a variant molecule (e.g., a variant protein). In some aspects, the methods disclosed herein can also comprise a processing step related to extracting a conformational state, for example, a metastable conformational state, from the low-dimensional conformational landscape of the variant molecule (e.g., a variant protein) to seed conformational dynamics generation (e.g. MD) of further variant molecules (e.g., variant proteins).

In some aspects, extracting a specific conformational state, for example, a metastable conformational state from the low-dimensional conformational landscape comprises clustering state conformational states according to structural similarity, e.g., the various metastable conformational states in the low-dimensional conformational landscape are the result of clustering conformational states. In some aspects, the average distance between the backbone atoms of identified conformational states is measured according to RMSD, wherein the RMSD is at least about 2 Å. In other aspects, different RMSD thresholds can be used, e.g., 1 Å, 1.5 Å, 2.5 Å, 3 Å, 3.5 Å, 4 Å, 4.5 Å, 5 Å, or any intermediate value.

By extracting specific conformational states (e.g., metastable conformations) from the conformational landscape (e.g., a MD conformational landscape) of the reference molecule (e.g., a reference protein that can be a wild type protein) to seed simulations (e.g., MD simulations) of variants, the overall simulation time required for comparing variants can be dramatically reduced. For example, in some aspects, the extraction of a collection of conformational states and subsequent simulation of variant molecule structures in these conformational states achieves a reduction in simulation times of at least about 10-fold. In some aspects, the extraction of a collection of conformational states and subsequent simulation of variant molecule structures in these conformational states achieves a reduction in simulation times of at least about 3-fold, at least about 4-fold, at least about 5-fold, at least about 6-fold, at least about 7-fold, at least about 8-fold, at least about 9-fold, at least about 10-fold, at least about 15-fold, or at least about 20-fold. In some aspects, the extraction of a collection of conformational states and subsequent simulation of variant molecule structures in these conformational states achieves a reduction in simulation times of about 3-fold, about 4-fold, about 5-fold, about 6-fold, about 7-fold, about 8-fold, about 9-fold, about 10-fold, about 15-fold, or about 20-fold. In some aspects, the extraction of a collection of conformational states and subsequent simulation of variant molecule structures in these states achieves a reduction in simulation times of about 3-fold to about 5-fold, about 4-fold to about 6-fold, about 5-fold to about 8-fold, about 6-fold to about 9-fold, about 5-fold about 10-fold, about 10-fold to about 15-fold, or about 15 fold to about 20-fold. All the improvements in performance are with respect to workflows that do not use representative metastable conformations to simulate the effect of variants.

Clustering states from the low-dimensional conformational landscape of the reference molecule (e.g., a reference protein) to seed simulations of a variant molecule (e.g., a variant protein) can be performed for example using k-mean clustering, hierarchical clustering, density-based clustering, DBSCAN, spectral clustering, Gaussian mixture models, or any combination thereof.

Clustering allows extracting multiple conformational states from a low-dimensional conformational landscape, which generally is in a range between 10 and 100 conformational states. In some aspects, at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 conformational states are extracted. In some aspects, about 5, about 10, about 15, about 20, about 25, about 30, about 35, about 40, about 45, about 50, about 55, about 60, about 65, about 70, about 75, about 80, about 85, about 90, about 95, or about 100 conformational states are extracted. In some aspects, between about 10 and about 20, between about 20 and about 30, between about 30 and about 40, between about 40 and about 50, between about 50 and about 60, between about 60 and about 70, between about 70 and about 80, between about 80 and about 90, or between about 90 and about 100 conformational states are selected.

In some aspects, the methods disclosed herein further comprise prioritizing the clustered conformational states prior to generating conformational dynamics (e.g. MD simulations) s In some aspects, clustered conformational states are prioritized according to cluster properties, e.g., distance from conformational landscape centroid, cluster occupancy, cluster distribution statistics, structural properties of the cluster, thermodynamics of cluster, or any combination thereof. In some aspects, the structural properties of the clustered conformational state are selected from the group consisting of bond angles (e.g., phi-psi dihedrals), inter-residue distances, surface accessibility, domain relative orientations, binding-site interactions, structural similarity to known protein conformations, and any combination thereof. In some aspects, the cluster distribution statistics are selected from the group consisting of Silhouette score, elbow score, Calinski-Harabasz Index, Rand Index, mutual information, homogeneity, completeness, V measure, Davies-Bouldin Index, and any combination thereof. In some, extracting a state from the low-dimensional conformational landscape comprises extracting a representative conformational state, e.g., a metastable conformational state. “Representative conformational state” comprises selecting a protein conformation that is closest in distance in the low dimensional representation of the conformational landscape to the centroid of the conformations of the selected conformational cluster.

### I.D Variant Molecule and Variant Molecule Structure Construction

The methods disclosed herein comprise generating a variant molecule (e.g., a variant protein) or set thereof and its corresponding variant molecule structure (e.g., a variant protein structure) by introducing at least one variant (e.g., a missense mutation) in a simulated protein conformation of the variant molecule in the extracted state. For example, a mutation identified in a sample obtained from a subject can be introduced in a MD simulated conformation disclosed herein to generate a variant protein structure. The effect such mutation has could be subsequently evaluated according to the methods disclosed herein. In other aspects, the variant (e.g., a mutation) can be a speculative variant introduced in a MD simulated conformation disclosed herein to gain knowledge about the functional and physiological effects and optionally their severity when, e.g., a particular domain, region, functional site (e.g., a ligand binding site) is mutated in a reference molecule (e.g., a reference protein) or variant molecule (e.g., a variant protein already comprising other variants). In some aspects, a collection of variant molecules (e.g., variant proteins) generated according to the methods of the present disclosure can be stored as a library.

In some aspects of the methods disclosed herein, at least one variant (e.g., a missense mutation) is introduced, e.g., in a reference molecule (e.g., reference protein, such as a wild type protein) or a variant thereof is a pathogenic variant (e.g., a pathogenic mutation). The term “pathogenic variant,” as used herein, refers to malignant variants related to the appearance of diseases. In some aspects, a pathogenic variant is a pathogenic mutation. The term “pathogenic mutation,” as used herein, refers to malignant mutations related to the appearance of diseases. These terms also refer to a variant or mutation that is risk-susceptible to disease, and the variant or mutation is capable of causing the corresponding disease phenotype, i.e., it is a genetic alteration that causes a disease or disorder and/or increases an individual's susceptibility or predisposition to that particular disease or disorder. However, it is not limited to the cases where the variant or mutation alone causes the disease or disorder. In some aspects, at least one variant or mutation (e.g., missense mutation) introduced, e.g., in a reference molecule (e.g., a reference protein such as a wild type protein) or in a variant thereof is a non-pathogenic (benign) variant or mutation, i.e., a variant or mutation that does not cause a disease or disorder in the subject or that doesn't predispose the subject to a particular disease or disorder. In some aspects, at least one variant (e.g., a mutation such as a missense mutation) introduced, e.g., in a reference molecule (e.g., a reference protein such as a wild type protein) or in a variant thereof is a variant or mutation of unknown significance, i.e., the empirical data available regarding the variant does not allow classifying such variant or mutation as either pathogenic or non-pathogenic One of the goals of the methods disclosed herein is classifying such variants or mutations of unknown relevance as either pathogenic or non-pathogenic as well determining, e.g., the likelihood that a variant or mutation of unknown relevance classified as pathogenic will cause a disease or disorder, and/or, for example, the severity of such disease or disorder.

In some aspects, the variant molecule (e.g., a protein) or set thereof comprises a protein missense variant observed in medical genetic testing. The term “genetic testing” as used herein refer to the analysis of DNA, RNA, or protein in a sample from an individual which determines, without limitation, the sequence of one, or more than one, gene within the sample, the presence or absence of one, or more than one, genetic marker, variance, mutation, polymorphism, or microsatellite sequence associated with a gene, the presence of one, or more than one, viral sequence, viral-like sequence, or repetitive sequence, a haplotype spanning one, or more than one, gene, the number of copies of one, or more than one, gene, the amount or characteristics of RNA or protein expressed from one, or more than one, gene, the arrangement of genes within the genome, the chromosome number, or integrity of chromosomes. In some aspects, the present disclosure concerns genetic tests useful in medicine for diagnosing genetic disease, determining an individual's propensity to multifactorial diseases, and predicting an individual's response to therapeutic drugs. Genetic tests have been developed for many inherited diseases including, but not limited to, Huntington's disease, cystic fibrosis, and phenylketonuria. Genetic tests have also been developed for genes that predispose to diseases including, but not limited to, atherosclerosis, heart failure, stroke, anemia, cancer, clotting disorders, dementia, endocrine diseases, and pulmonary diseases. Genetic tests have also been described which predict the pharmacokinetic and pharmacodynamic characteristics of many drugs including, but not limited to, drugs for the treatment of elevated cholesterol, drugs to treat cancer, drugs to reduce hypertension, and drugs to treat dementia. Despite the potentially great impact of genetic tests on health care, the utilization of genetic tests is currently very limited. To date, only a fraction of the genes in the genome have been characterized, and only a small subset of variations that are predictive of disease or its response to therapy have been described.

In some aspects, the variant molecule structure (e.g., a variant protein structure) or set thereof used in the methods disclosed herein or included in a library assembled according to the methods disclosed herein is generated in silico using a backbone-dependent rotamer library. Other methods that could be used to generate these variant molecule structures in silico include, e.g., FoldX or Rosetta. The term “rotamer” refers to a combination of torsion angles describing the geometry of an amino acid side chain.

Rotamer libraries are known to those of skill in the art and can be obtained from a variety of sources, including the internet. Rotamers are low energy side-chain conformations. The use of a library of rotamers allows for the modeling of a structure to try the most likely side-chain conformations, saving time and producing a structure that is more likely to be correct.

In some aspects, the in silico generation of a variant molecule structure (e.g., a variant protein structure) to be used in the methods disclosed herein comprises identifying side chain conformations (rotamers) with the existing backbone dihedral angles of the variant molecule structure (e.g., variant protein) at the variant position in the backbone-dependent rotamer library.

In some aspects, the method to generate in silico a variant molecule structure further comprises testing whether the introduction of a rotamer identified from a backbone-dependent rotamer library causes a steric clash with nearby residues. In some aspects, testing is conducted until a rotamer is identified that minimizes steric clash. In some aspects, the method to generate in silico a variant molecule structure of the present disclosure (e.g., a variant protein structure) further comprises conducting a minimization and equilibration simulation to regularize the geometry of the variant molecule structure (e.g., variant protein structure) and to decrease the energetic impact of introducing the variant (e.g., a missense mutation) in the variant molecule structure.

### I.E Predictive Modeling

In some aspects, the methods disclosed herein comprise a processing step comprising training a machine-learning (ML) predictive model using the low-dimensional representation of the conformational landscape of the reference molecule (e.g., a reference protein) and a variant molecule (e.g., a variant protein) using clinical data as training labels, wherein the predictive model classifies or scores the variant (e.g., protein missense mutation) effects on the reference molecule (e.g., a reference protein). Thus, in some aspects, the training set of the ML method of choice comprises both MD simulation-related data as well as other sets of data, e.g., clinical data.

The phrase “training set” means a set of observations that is the subject of fitting of one or more models and in which the model is built based thereon. For example, in the case of a predictive model in which protein activity is predicted, the training set typically includes complete or partial protein sequence information along with an activity value for each protein in the library. In some cases, multiple activity types (e.g., rate constant data and thermal stability data) are provided together in a training set. In addition to data related to protein sequence, protein structure, protein dynamics, or protein activity, a training set may comprise, for example, clinical data.

In some aspects, the clinical data used as training labels in a training set comprises any parameters related to a patient developing or at risk of developing a disease or condition related to the target protein or a variant thereof, symptoms, sequelae, treatments of the disease or condition, outcomes of treatments, etc. For example, clinical data that can be used as training labels comprises, e.g., biomarker status (e.g., presence or absence of a certain biomarker or its expression level, e.g., RNA or protein expression level), biometric data, lifestyle-related data, response to treatments, symptoms of the disease or conditions, protein expression data, type of treatment administered, dosage, dosage regimen, administration route, presence or absence of co-therapies, response to the therapy, age, body weight, gender, ethnicity, or any combination thereof. In some aspects, data that can be used as training labels comprises, e.g., NCBI ClinVar database submissions, INVITAE™ clinical interpretations, data from other experimental or computational models.

In some aspects, a training label results from the combination of individually identified clinical data parameters, e.g., several protein expression values, biometric parameters, etc. can be combined into an index (e.g., BMI) or a score (e.g., a ratio between the expression levels of two proteins).

In some aspects, the classification of the effect of a variant (e.g., a missense mutation) on the reference molecule (e.g., a reference protein) comprises the calculation of a predicted pathogenicity probability based on the output of the ML predictive model. In some aspects, the probability is calculated as a confidence score from the ML predictive model. In some aspects, the ML output of the methods disclosed herein can predict the specific phenotypic disruption compared to the reference molecule (e.g., a reference protein) and rank the relative severity of the disruption.

In general, the computer-implemented methods disclosed herein generate a predicted pathogenicity score, which is a confidence score of how likely a model predicts a variant to be pathogenic. In some aspects, a performance threshold is applied to the pathogenic scores to determine whether to assign evidence that a specific variant is pathogenic or benign.

In the context of the present disclosure, it is to be understood that the term predictive model includes one or more predictive models, or combinations of predictive models, which can belong to the same or different classes, e.g., predictive model generated using different ML techniques or using different sets of clinical data as training labels, wherein the term predictive model is used to describe the output of a specific model. For example, in addition to ML predictive models predicting likelihood and/or severity of phenotypic disruption, other ML predictive models may predict binding of the generated variants to specific drugs, candidate therapies, outcomes related to treatments with specific therapies, or any combination thereof.

In some aspects, the preprocessing of the training set prior to generating the ML predictive model comprises feature selection, and optionally feature normalization. In some aspects, the ML model is trained using hyperparameter tuning. In ML, hyperparameter optimization or tuning is the problem of choosing a set of optimal hyperparameters for a learning algorithm. A hyperparameter is a parameter whose value is used to control the learning process. By contrast, the values of other parameters (typically node weights) are learned. The same kind of ML predictive model can require different constraints, weights or learning rates to generalize different data patterns. These measures are called hyperparameters, and have to be tuned so that the model can optimally solve the ML problem. Hyperparameter optimization finds a tuple of hyperparameters that yields an optimal model, which minimizes a predefined loss function on given independent data. The objective function takes a tuple of hyperparameters and returns the associated loss. Cross-validation is often used to estimate this generalization performance.

In some aspects, an ML predictive model disclosed herein is obtained by the application of a machine-learning technique selected from the group consisting of Logistic Regression, Random Forest, Artificial Neural Network (ANN), Support Vector Machine (SVM), XGBoost (XGB; an implementation of gradient boosted decision trees designed for speed and performance), Glmnet (a package that fits a generalized linear model via penalized maximum likelihood), cforest (implementation of the random forest and bagging ensemble algorithms utilizing conditional inference trees as base learner), Classification and Regression Trees for Machine-learning (CART), Treebag (bagging, i.e., bootstrap aggregating, algorithm to improve model accuracy in regression and classification problems which building multiple models from separated subsets of train data, and constructs a final aggregated model), K-Nearest Neighbors (kNN), or a combination thereof. In a specific aspect, the ML predictive models disclosed herein are obtained by the application of at least one ANN, e.g., one, two, three, four, or five ANNs.

### I.E.i Logistic Regression ML Predictive Models

In some aspects, a machine-learning (ML) predictive model of the present disclosure is generated using Logistic Regression. Logistic Regression often is regarded as one of the best predictors on small datasets. However, Tree-based models (e.g., Random Forest, ExtraTrees) and ANNs can uncover latent interactions among features. When there is little interaction, though, Logistic Regression and more complex models have similar performance. In some aspects of the present disclosure, the ML predictive model is generated using Logistic Regression. In one aspect, for the logistic regression model, the following hyperparameters were tuned: stopping criterion tolerance, penalty, solving algorithm (e.g. LIBLINEAR, stochastic average gradient).

### I.E.ii Random Forests ML Predictive Models

In some aspects, a machine-learning (IL) predictive model of the present disclosure is generated using a Random Forest (RF). RF or random decision forest is an ensemble learning method for classification, regression and other tasks that operates by constructing a multitude of decision trees at training time. For classification tasks, the output of the RF is the class selected by most trees. RFs adjust for decision trees' habit of overfitting to their training set. RFs generally outperform decision trees. RF algorithms use bootstrap samples to generate individual decision trees. The trees are grown by selecting a random subsample of the independent variables at each node and selecting the variable that produces the best outcome.

In one specific aspect, the predictive model is allowed to optimize validation performance (using a class-weighted F1-score as a tuning metric) between both logistic regression and random forests. For random forest, the following hyperparameters were tuned: number of estimators, split criterion, maximum tree depth, minimum sample to split a tree, minimum samples in leaf nodes, and maximum number of features.

### I.E.iii Artificial Neural Network ML Predictive Models

In some aspects, a machine-learning (IL) predictive model of the present disclosure is generated using an Artificial Neural Network (ANN). A “neural network” is a model that includes interconnected groups of processing elements or “neurons” that process information using a connection approach to computation. Neural networks are used to model complex relationships between input and output values and/or to find data patterns. Most neural networks process data in a nonlinear, distributed, and parallel manner. In most cases, the neural network is an adaptive system that changes its structure during the learning phase. Functions are executed collectively and in parallel by processing elements rather than using explicit descriptions of sub-tasks to which various units are assigned.

Neural networks are described in Duda et al., 2001, Pattern Classification, Second Edition, John Wiley & Sons, Inc., New York; and Hastie et al., 2001, The Elements of Statistical Learning, Springer-Verlag, New York, which are herein incorporated by reference in their entireties In some aspects, a neural network of the present disclosure, e.g., a back-propagation neural network (see, for example Abdi, 1994, “A neural network primer”, J. Biol System. 2, 247-283, which is herein incorporated by reference in its entirety) can contain an input layer, a hidden layer, and an output layer. In some aspects, the neural network can be implemented using the EasyNN-Plus version 4.0 g software package (Neural Planner Software Inc.), scikit-learn (scikit-learn.org), PyTorch (github.com/pytorch/pytorch), TensorFlow (github/tensorflow/tensorflow), Keras, Torch, Flux, Caffe, CNTK (Microsoft Cognitive Toolkit), or any other machine learning package or program known in the art.

In some aspects, the ML predictive model comprises a model derived from an ANN. In some aspects, the ANN is a feed-forward neural network. In some aspects, the ANN is a single-layer perceptron network

In some aspects, the ANN is a multi-layer perceptron (MLP). In some aspects, the activation function is a sigmoid function. In some aspects, the activation function is a rectifier linear unit (ReLU) or a variant thereof, e.g., a noisy ReLU, a leaky ReLU, a parametric ReLU, or an exponential LU. In some aspects, the MLP comprises three or more layers (an input and an output layer with one or more hidden layers) of nonlinearly-activating nodes. Its multiple layers and non-linear activation distinguish MLP from a linear perceptron. It can distinguish data that is not linearly separable. Since MLPs are fully connected, each node in one layer connects with a certain weight wᵢⱼ to every node in the following layer. Learning occurs in the perceptron by changing connection weights after each piece of data is processed, based on the amount of error in the output compared to the expected result. This is an example of supervised learning, and is carried out through backpropagation.

In some aspects, the MLP has three layers. In other aspects, the MLP has more than three layers. In some aspects, the MLP has a single hidden layer. In other aspects, the MLP has more than one hidden layer.

In some aspects, the input layer comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 neurons. In some aspects, the input layer comprises between 70 and 100 neurons. In some aspects, the input layer comprises between 70 and 80 neurons. In some aspects, the input layer comprises between 80 and 90 neurons. In some aspects, the input layer comprises between 90 and 100 neurons. In some aspects, the input layer comprises between 70 and 75 neurons. In some aspects, the input layer comprises between 75 and 80 neurons. In some aspects, the input layer comprises between 80 and 85 neurons. In some aspects, the input layer comprises between 85 and 90 neurons. In some aspects, the input layer comprises between 90 and 95 neurons. In some aspects, the input layer comprises between 95 and 100 neurons. In some aspects, the input layer comprises between at least about 1 to at least about 5, between at least about 5 and at least about 10, between at least about 10 and at least about 15, between at least about 15 and at least about 20, between at least about 20 and at least about 25, between at least about 25 and at least about 30, between at least about 30 and at least about 35, between at least about 35 and at least about 40, between at least about 40 and at least about 45, between at least about 45 and at least about 50, between at least about 50 and at least about 55, between at least about 55 and at least about 60, between at least about 60 and at least about 65, between at least about 65 and at least about 70, between at least about 70 and at least about 75, between at least about 75 and at least about 80, between at least about 80 and at least about 85, between at least about 85 and at least about 90, between at least about 90 and at least about 95, between at least about 95 and at least about 100, between at least about 100 and at least about 105, between at least about 105 and at least about 110, between at least about 110 and at least about 115, between at least about 115 and at least about 120, between at least about 120 and at least about 125, between at least about 125 and at least about 130, between at least about 130 and at least about 135, between at least about 135 and at least about 140, between at least about 140 and at least about 145, or between at least about 145 and at least about 150 neurons. In some aspects, the input layer comprises between at least about 1 and at least about 10, between at least about 10 and at least about 20, between at least about 20 and at least about 30, between at least about 30 and at least about 40, between at least about 40 and at least about 50, between at least about 50 and at least about 60, between at least about 60 and at least about 70, between at least about 70 and at least about 80, between at least about 80 and at least about 90, between at least about 90 and at least about 100, between at least about 100 and at least about 110, between at least about 110 and at least about 120, between at least about 120 and at least about 130, between at least about 130 and at least about 140, or between at least about 140 and at least about 150 neurons. In some aspects, the input layer comprises between at least about 1 and at least about 20, between at least about 20 and at least about 40, between at least about 40 and at least about 60, between at least about 60 and at least about 80, between at least about 80 and at least about 100, between at least about 100 and at least about 120, between at least about 120 and at least about 140, between at least about 10 and at least about 30, between at least about 30 and at least about 50, between at least about 50 and at least about 70, between at least about 70 and at least about 90, between at least about 90 and at least about 110, between at least about 110 and at least about 130, or between at least about 130 and at least about 150 neurons. In some aspects, the input layer comprises more than about 1, more than about 5, more than about 10, more than about 15, more than about 20, more than about 25, more than about 30, more than about 35, more than about 40, more than about 45, more than about 50, more than about 55, more than about 60, more than about 65, more than about 70, more than about 75, more than about 80, more than about 85, more than about 90, more than about 95, more than about 100, more than about 105, more than about 110, more than about 115, more than about 120, more than about 125, more than about 130, more than about 135, more than about 140, more than about 145, or more than about 150 neurons. In some aspects, the input layer comprises less than about 1, less than about 5, less than about 10, less than about 15, less than about 20, less than about 25, less than about 30, less than about 35, less than about 40, less than about 45, less than about 50, less than about 55, less than about 60, less than about 65, less than about 70, less than about 75, less than about 80, less than about 85, less than about 90, less than about 95, less than about 100, less than about 105, less than about 110, less than about 115, less than about 120, less than about 125, less than about 130, less than about 135, less than about 140, less than about 145, or less than about 150 neurons. In some aspects, a weight is applied to the input of each one of the neurons in the input layer. In some aspects, the ANN comprises a single hidden layer. In some aspects, the ANN comprises 1, 2, 3, 4, 5, 6, 7, 8, 9 or 10 hidden layers. In some aspects, the single hidden layer comprises 1, 2, 3, 4, 5, 6, 7, 8, 9, or 10 neurons. In some aspects, the single hidden layer comprises at least 1, at least 2, at least 3, at least 4, at least 5, at least 6, at least 7, at least 8, at least 9, or at least 10 neurons. In some aspects, the single hidden layer comprises less than 10, less than 9, less than 8, less than 7, less than 6, less than 5, less than 4, or less than 3 neurons. In some aspects, the single hidden layer comprises two neurons. In some aspects, the single hidden layer comprises three neurons. In some aspects, the single hidden layer comprises four neurons. In some aspects, the single hidden layer comprises five neurons. In some aspects, a bias is applied to the neurons in the hidden layer. In some aspects, the classification of the output layer is binary. In some aspects, the classification of the output layer is normalized to a probability distribution over predicted output classes (e.g., pathological vs. non-pathological variant), and the components will add up to 1, so that they can be interpreted as probabilities. In some aspects, the classification of the output layer values into n phenotype classes is supported by applying a logistic regression function. In some aspects, the classification of the output layer values into n phenotype classes is supported by applying a logistic regression predictive model, e.g., the Softmax function. Softmax assigns decimal probabilities to each class that adds up to 1.0. In some aspects, the use of a logistic regression predictive model such as the Softmax function helps training converge more quickly. In some aspects, the logistic regression predictive model comprising a Softmax function is implemented through a neural network layer just before the output layer. In some aspects, such neural network layer just before the output layer has the same number of nodes as the output layer. In some aspects, various cut-offs are applied to the results of the logistic regression predictive model (e.g., Softmax function) depending on the particular dataset used (see, e.g., cut-offs applied to select a particular type of pathological phenotype, or a particular degree of severity of a specific pathological phenotype). Thus, applying different sets of cut-offs can classify the phenotypic impact of a missense variant not only in a binary fashion (e.g., pathogenic vs. non-pathogenic), but also classify or stratify the phenotypic impact of a missense variant according to, e.g., degree of severity. In some aspects of the present disclosure, the ANN is a multi-layer perceptron with independent state NNs with at least one hidden layers that are then integrated into a combined NN with the number of nodes equal to the number of nodes per each state NN, and that has at least one hidden layer and a final Softmax output layer. The multi-layer perceptron can use ReLU or tanh activation, SGD or Adam optimization, NLL, Hinge or cross entropy loss function, and dropout layers to prevent overfitting.

In some aspects, the quality of the resulting MIL predictive models is evaluated. Accordingly, in one aspect of the disclosure, each predictive model is evaluated for its ability to properly characterize the phenotypic impact of each variant molecule (e.g., variant protein or variant gene) of the training population using methods known to a person skilled in the art. For example, one can evaluate the predictive model using cross validation, Leave One Out Cross Validation (LOOCV), n-fold cross validation, or jackknife analysis using standard statistical methods. In another aspect, each predictive model is evaluated for its ability to properly characterize variant molecules (e.g., variant proteins or variant genes) which were not used to generate the predictive model. In some aspects, one can train the predictive model using one dataset, and evaluate the predictive model on another distinct dataset. In one aspect, the method used to evaluate the predictive model for its ability to properly characterize each variant molecule (e.g., variant protein or variant gene) of the training set is a method that evaluates the predictive model's sensitivity (TPF, true positive fraction) and 1-specificity (FPF, false positive fraction). In one aspect, the method used to test the predictive model is Receiver Operating Characteristic (“ROC”) which provides several parameters to evaluate both the sensitivity and specificity of the result of the predictive model generated, e.g., a predictive model derived from the application of an ANN.

In some aspects, the metrics used to evaluate the predictive model for its ability to properly characterize each variant molecule (e.g., variant protein or variant gene) of the training population comprise classification accuracy (ACC), Area Under the Receiver Operating Characteristic Curve (AUC ROC), Sensitivity (True Positive Fraction, TPF), Specificity (True Negative Fraction, TNF), Positive Predicted Value (PPV), Negative Predicted Value (NPV), or any combination thereof. In one specific aspect, the metrics used to evaluate the predictive model for its ability to properly characterize each variant molecule (e.g., variant protein or variant gene) of the training population are classification accuracy (ACC), Area Under the Receiver Operating Characteristic Curve (AUC ROC), Sensitivity (True Positive Fraction, TPF), Specificity (True Negative Fraction, TNF), Positive Predicted Value (PPV), and Negative Predicted Value (NPV).

### II. Clinical, Diagnostic, and Drug Design Applications

In some aspects, the computational methods to determine the phenotypic impact of a variant (e.g., a mutation) disclosed herein can further comprise (i) obtaining and processing a sample from a subject to determine the presence of any variants (e.g., missense variants) of a reference molecule (e.g., a variant protein), for example, by obtaining a tissue sample, processing the tissue sample to obtain RNA or DNA, and sequence the RNA or DNA for example using NGS methods), and/or (ii) the formulation of a treatment plan, which can include, e.g., the administration of a therapy or treatment to the subject.

As used herein, the terms “therapy” and “therapeutic” include prophylaxis and prophylactic and encompass prevention as well as amelioration of symptoms associated with a disease or disorder, inhibition or delay of progression of a disease or disorder, and treatment of a disease or disorder.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease or enhancing overall survival. Treatment can be of a subject having a disease or a subject who does not have a disease (e.g., for prophylaxis). As used here, the terms “treat,” “treating,” and “treatment” refer to the administration of an effective dose or effective dosage.

As used herein, the term “disease” means any phenotype or phenotypic trait of concern, including by way of example a disease or disease state, a predisposition or susceptibility to a disease, or an abnormal drug response. Illustrative and non-limiting examples of disease states include cancer, high cholesterol levels, congestive heart failure, hypertension, diabetes, glucose intolerance, depression, anxiety, infectious disease, toxic states, drug therapy side effects, inefficacy of drug therapy, alcoholism, addiction, trauma, etc. In general, a disease is particular distinctive process in the body with a specific cause and characteristic symptoms. As used herein, the term “disorder” refers to an irregularity, disturbance, or interruption of normal functioning of normal functions of the body.

“Subject” generally means a biological organism with associated and sequence information, and optionally phenotypic information, available for analysis. As used herein, the term “subject” includes any human or nonhuman animal. The terms, “subject” and “patient” are used interchangeably herein. The term “nonhuman animal” includes, but is not limited to, vertebrates such as dogs, cats, horses, cows, pigs, boar, sheep, goat, buffalo, bison, llama, deer, elk and other large animals, as well as their young, including calves and lambs, and to mice, rats, rabbits, guinea pigs, primates such as monkeys and other experimental animals. Within animals, mammals are preferred, most preferably, valued and valuable animals such as domestic pets, racehorses and animals used to directly produce (e.g., meat) or indirectly produce (e.g., milk) food for human consumption, although experimental animals are also included. In specific aspects, the subject is a human. Thus, the present disclosure is applicable to clinical, veterinary and research uses.

Variants (e.g., missense mutations) identified for example in a variant molecule (e.g., a variant gene) from a subject can be used as inputs to generate in silico variant molecule structures (variant protein structure) which could be used, e.g., the determine the phenotypic impact of the mutation, to generate MD simulated structures to incorporate into a library or to store for future analysis, to expand the ML training set in order to improve the ML predictive models, or any combination thereof.

In some aspects, samples for genetic testing to be used to identify variants of a reference molecule (e.g., a reference protein), for example, be requested by a healthcare provider (e.g., a doctor) or healthcare benefits provider. In some aspects, samples for genetic testing can be obtained and/or processed by the same or a different healthcare provider (e.g., a nurse, a hospital) or a clinical laboratory, and after processing, the results can be forwarded to the original healthcare provider or yet another healthcare provider, healthcare benefits provider, or a facility that would conduct the computational analysis of the variant-related information obtained from the subject (e.g., conduct part or entire workflow disclosed herein). Based on the results from the methods disclosed herein, a decision regarding whether to administer a treatment could be made.

In some aspects, the term “administering” can also comprise commencing a therapy, discontinuing or suspending a therapy, temporarily suspending a therapy, or modifying a therapy (e.g., increasing dosage or frequency of doses, or adding one of more therapeutic agents in a combination therapy).

As used herein, the term “healthcare provider” refers to individuals or institutions that directly interact with and administer to living subjects, e.g., human patients. Non-limiting examples of healthcare providers include doctors, nurses, technicians, therapist, pharmacists, counselors, alternative medicine practitioners, medical facilities, doctor's offices, hospitals, emergency rooms, clinics, urgent care centers, alternative medicine clinics/facilities, and any other entity providing general and/or specialized treatment, assessment, maintenance, therapy, medication, and/or advice relating to all, or any portion of, a patient's state of health, including but not limited to general medical, specialized medical, surgical, and/or any other type of treatment, assessment, maintenance, therapy, medication and/or advice.

As used herein, the term “clinical laboratory” refers to a facility for the examination or processing of materials derived from a living subject, e.g., a human being. Non-limiting examples of processing include biological, biochemical, serological, chemical, immunohematological, hematological, biophysical, cytological, pathological, genetic, or other examination of materials derived from the human body for the purpose of providing information, e.g., for the diagnosis, prevention, or treatment of any disease or impairment of, or the assessment of the health of living subjects, e.g., human beings. These examinations can also include procedures to collect or otherwise obtain a sample, prepare, determine, measure, or otherwise describe the presence or absence of various substances in the body of a living subject, e.g., a human being, or a sample obtained from the body of a living subject, e.g., a human being.

As used herein, the term “healthcare benefits provider” encompasses individual parties, organizations, or groups providing, presenting, offering, paying for in whole or in part, or being otherwise associated with giving a patient access to one or more healthcare benefits, benefit plans, health insurance, and/or healthcare expense account programs.

A healthcare provider can implement or instruct another healthcare provider or patient to perform the following actions: obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, administer a therapy, commence the administration of a therapy, cease the administration of a therapy, continue the administration of a therapy, temporarily interrupt the administration of a therapy, increase the amount of an administered therapeutic agent, decrease the amount of an administered therapeutic agent, continue the administration of an amount of a therapeutic agent, increase the frequency of administration of a therapeutic agent, decrease the frequency of administration of a therapeutic agent, maintain the same dosing frequency on a therapeutic agent, replace a therapy or therapeutic agent by at least another therapy or therapeutic agent, combine a therapy or therapeutic agent with at least another therapy or additional therapeutic agent.

In some aspects, a healthcare benefits provider can authorize or deny, for example, collection of a sample, processing of a sample, submission of a sample, receipt of a sample, transfer of a sample, analysis or measurement a sample, quantification of a sample, provision of results obtained after analyzing/measuring/quantifying a sample, transfer of results obtained after analyzing/measuring/quantifying a sample, comparison/scoring of results obtained after analyzing/measuring/quantifying one or more samples, transfer of the comparison/score from one or more samples, administration of a therapy or therapeutic agent, commencement of the administration of a therapy or therapeutic agent, cessation of the administration of a therapy or therapeutic agent, continuation of the administration of a therapy or therapeutic agent, temporary interruption of the administration of a therapy or therapeutic agent, increase of the amount of administered therapeutic agent, decrease of the amount of administered therapeutic agent, continuation of the administration of an amount of a therapeutic agent, increase in the frequency of administration of a therapeutic agent, decrease in the frequency of administration of a therapeutic agent, maintain the same dosing frequency on a therapeutic agent, replace a therapy or therapeutic agent by at least another therapy or therapeutic agent, or combine a therapy or therapeutic agent with at least another therapy or additional therapeutic agent.

In addition, a healthcare benefits provider can, e.g., authorize or deny the prescription of a therapy, authorize or deny coverage for therapy, authorize or deny reimbursement for the cost of therapy, determine or deny eligibility for therapy, etc.

In some aspects, a clinical laboratory can, for example, collect or obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, or other related activities.

The methods, systems, tangible computable readable devices, etc. disclosed herein can be used to classify a variant (e.g., a protein missense mutation) in a variant molecule (e.g., a variant protein), e.g., as pathogenic or non-pathogenic, and such classification can be used in turn to (i) treat a patient, (ii) selected a patient for treatment, (iii) commence a treatment, (iv) discontinue a treatment, (v) interrupt a treatment, (vi) modify a treatment, or (vii) any combination thereof.

The present disclosure provides a method to (i) treat a patient, (ii) selected a patient for treatment, (iii) commence a treatment, (iv) discontinue a treatment, (v) interrupt a treatment, (vi) modify a treatment, or (vii) any combination thereof, comprising applying the methods, systems, tangible computable readable devices, etc. disclosed herein.

The present disclosure provides also a method of determining whether to treat a subject with a specific therapy comprising applying the methods, systems, tangible computable readable devices, etc. disclosed herein. For example, determining that a variant molecule (e.g., a variant protein) is pathogenic and can result in the manifestation of certain symptoms may be used to select a specific therapy that would treat, or ameliorate such symptoms.

The present disclosure also provides a personalized medicine treatment comprising (i) identifying a specific variant molecule (e.g., a variant protein) in a subject, (ii) determining the phenotypic impact of the specific variant molecule (e.g., a variant protein) using the methods disclosed herein, e.g., a ML predictive model for diagnosing and/or predicting the phenotypic impact of a variant molecule (e.g., variant protein such as a missense variant protein), the workflows used generate such ML predictive models, or associates libraries (e.g., libraries of precomputed MD models, libraries of low-dimensionality conformational landscapes, libraries of low-dimensionality states, or libraries of ML predictive models), and (iii) selecting a treatment to treat, prevent, or ameliorate a disease or condition associated with the variant molecule.

In one aspect, the methods disclosed herein include making a diagnosis, which can be a differential diagnosis, based at least in part, e.g., on the classification or prediction of the phenotypic impact by a predictive model of the present disclosure, of a variant molecule (e.g., a variant protein) in a subject, wherein the variant molecule (e.g., a variant protein) has been classified through the application of a ML predictive model disclosed herein. This diagnosis can be recorded in a patient medical record. The medical record can be in paper form and/or can be maintained in a computer-readable medium. The medical record can be maintained by a laboratory, physician's office, a hospital, a healthcare maintenance organization, an insurance company, and/or a personal medical record website.

Factors known in the art for diagnosing and/or suggesting, selecting, designating, recommending or otherwise determining a course of treatment for a patient or class of patients having or at risk of a having a disease or disorder can be employed, e.g., in combination with measurements of the reference or variant molecule expression (e.g., the RNA or protein expression levels of a reference or variant protein), and in combination with the pathogenicity classifications, predictive models, or other outputs from the method disclosed herein.

The present disclosure also provides portfolios of precomputed variant molecule structures (e.g., variant protein structures) or precomputed pathogenicity scores for variant molecules (e.g., variant proteins). Such portfolios can be generated using the methods disclosed herein.

In some aspects, a diagnosis, based on the application of a ML predictive model disclosed herein can be recorded on or in a medical alert article such as a card, a worn article, and/or a radio-frequency identification (RFID) tag. As used herein, the term “worn article” refers to any article that can be worn on a subject's body, including, but not limited to, a tag, bracelet, necklace, or armband.

In some aspects, the methods disclosed herein, e.g., methods to generate a ML predictive model for diagnosing and/or predicting the phenotypic impact of variants (e.g., missense variants), the workflows used generate such ML predictive models, and/or associated libraries, e.g., libraries of precomputed conformational dynamics (e.g., MD) models, libraries of low-dimensionality conformational landscapes, libraries of low-dimensionality states, or libraries of ML predictive models, can be used to design or select a therapeutic agent to treat, prevent, or ameliorate a disease or condition associated with the reference molecule (e.g., a reference protein) or a variant thereof. For example, a pre-computed MD simulated structure of a reference molecule or a variant molecule may be used by a docking program to identify novel or existing drugs that may interact optimally with the reference molecule or a variant molecule.

In some aspects, portfolios of precomputed variant molecules (e.g., precomputed protein structures), precomputed pathogenicity scores for variant molecules, models or libraries of models generated as disclosed herein, can be used to select patients for clinical trials.

### III. Reference and Variant Molecules and Indications

The methods described herein can be used with missense variant protein for providing information for predicting, diagnosing, and treating various diseases, e.g., cancer, neurodegenerative diseases, and genetic diseases. Examples of cancers include breast cancer, ovarian cancer, lung cancer, brain cancer, leukemia, prostate cancer, and pancreatic cancer. Examples of neurodegenerative diseases include Alzheimer's, Parkinson's, and Huntington's disease. Genetic diseases include diseases and disorders that are caused by mutations in a single gene (monogenic disease) or in multiple genes (polygenic disease), by a combination of mutations and environmental factors, or by damage to chromosomes (changes in the number or structure of entire chromosomes). With monogenic diseases, the mutation may be present on one or both chromosomes. Examples of monogenic diseases include Marfan syndrome, Tay-Sachs disease, sickle cell anemia, cystic fibrosis, and polycystic kidney disease. Polygenic diseases are caused by a combination of small inherited variations in multiple genes. Examples of polygenic diseases include cancers, heart diseases, diabetes, and autoimmune diseases.

In some aspects, a reference molecule of the present disclosure can be a gene disclosed below, or a polynucleotide encoding a domain thereof. In other aspects, a reference molecule of the present disclosure can be a protein encoded by a gene disclosed below, or a domain thereof. In some aspects, a reference molecule can be a complex comprising at least one protein encoded by a gene disclosed below, or a domain thereof.

In some aspects, a variant molecule of the present disclosure can be a gene disclosed below, comprising one or more variants (e.g., mutations) with respect to the gene's canonical sequence, or a polynucleotide encoding a domain thereof. In other aspects, a variant molecule of the present disclosure can be a protein encoded by a gene disclosed below comprising one or more variants (e.g., mutations) with respect to the protein's canonical sequence, or a domain thereof. In some aspects, a variant molecule can be a complex comprising at least one protein encoded by a gene disclosed below, or a domain thereof.

The full names and sequences of the genes disclosed below as well as those of their corresponding proteins can be found in their corresponding Uniprot (www.uniprot.org), and RefSeq (www.ncbi.nlm.nih.gov/refseq) entries in the latest database releases available on Sep. 9, 2021, which are herein incorporated by reference in their entireties.

In some aspects, the reference molecule (e.g., a reference protein) is selected from the group consisting of SLC12A3, MLH1, GAA, MSH2, LDLR, COL6A2, TP53, GLDC, KCNQ1, COL6A1, CAPN3, MUTYH, VHL, TGM1, IGHMBP2, PYGM, ABCD1, PMS2, CLCN1, CHEK2, MFN2, WFS1, SLC22A5, and CPT2. In some aspects, the reference molecule (e.g., a reference protein) is MSH2, a domain thereof, or a complex comprising MSH2, e.g., MSH2/MSH6.

Specific proteins that can be used as reference molecule to predict the phenotypic impact caused by variants thereof are disclosed below. In some aspects, the reference molecule (e.g., a reference protein) or a variant thereof (i.e., a variant molecule such as a missense variant protein), relates to or is associate to a mendelian disorder, wherein the reference molecule or a variant thereof is encoded by a gene selected from the group consisting of BRCA1, BRCA2, APOB, LDLR, PCSK9, SCN5A, APC, MLH1, MSH2, MSH6, STK11, MUTYH, MYH7, LMNA, MYBPC3, TNNI3, TNNT2, KCNQ1, KCNH2, SDHB, ACTA2, MYH11, VHL, RET, SDHAF2, SDHC, SDHD, TP53, TSC1, TSC2, NF2, PTEN, RB1, RYR1, GLA, RYR2, TGFBR1, TGFBR2, ACTC1, CACNA1S, COL3A1, DSC2, DSG2, DSP, FBN1, MEN1, MYL2, MYL3, PKP2, PMS2, PRKAG2, SMAD3, TMEM43, TPM1, WT1, BMPR1A, SMAD4, ATP7B, and OTC. The term “associated with” means that there has been a link or correlation between the items discussed. For example, a particular protein or gene might be associated with a disease. This would mean that the protein or gene has been linked or is correlated with the presence of the disease. It can also mean that the protein or gene has been shown to be wholly or in part causative of the disease.

In some aspects, genes can appear to be driver genes, based on the results of the training of the machine-learning model (trained predicted model). The term “driver gene” as used herein, refers to a gene that includes a driver gene mutation. In some aspects, a driver gene is a gene in which one or more acquired mutations, e.g., driver gene mutations, can be causally linked, e.g., to cancer progression. In some aspects, a driver gene can modulate one or more cellular processes including cell fate determination, cell survival and genome maintenance. A driver gene can be associated with (e.g., can modulate) one or more signaling pathways, e.g., a TGF-beta pathway, a MAPK pathway, a STAT pathway, a PI3K pathway, a RAS pathway, a cell cycle pathway, an apoptosis pathway, a NOTCH pathway, a Hedgehog (HH) pathway, a APC pathway, a chromatin modification pathway, a transcriptional regulation pathway, a DNA damage control pathway, or a combination thereof. Exemplary driver genes include oncogenes and tumor suppressors. In some aspects, a driver gene provides a selective growth advantage to the cell in which it occurs. In some aspects, a driver gene provides a proliferative capacity to the cell in which it occurs, e.g., allows for cell expansion, e.g., clonal expansion. In some aspects, a driver gene is an oncogene. In some aspects, a driver gene is a tumor suppressor gene (TSG).

In some aspects, the reference molecule (e.g., a reference protein) or a variant molecule (e.g., missense variant protein) is a “cancer driver,” wherein the reference molecule or variant molecule is encoded by a gene selected from the group consisting of TP53, PIK3CA, ARID1A, RB1, PTEN, KRAS, BRAF, CDKN2A, NRAS, FBXW7, STAG2, NFE2L2, NF1, IDH1, ATM, PIK3R1, CASP8, HRAS, MLL2, SF3B1, ERBB2, CREBBP, AKT1, HLA-A, CTCF, ERBB3, CTNNB1, RUNX1, MYD88, SMARCA4, EP300, SETD2, SMARCB1, EGFR, TBL1XR1, U2AF1, EZH2, RAC1, MLL3, IL7R, CD79B, POU2AF1, MAP2K1, PTPN11, CCND1, MAP2K4, TCF7L2, KIT, CDK4, FOXA1, TSC1, FAT1, WT1, BCOR, XPO1, PRDM1, KEAP1, NSD1, PPP2R1A, CDKN1B, ASXL1, MET, RPL5, MYCN, TNFRSF14, FLT3, ALK, KDM5C, KDM6A, APC, PBRM1, STK11, RAD21, EZR, SPOP, TET2, PHF6, IRF4, DDX5, CCDC6, HIST1H3B, CARD11, IDH2, MLL, FGFR2, CDK12, ERCC2, B2M, MED12, CEBPA, NOTCH1, BRCA1, MAP3K1, VHL, DNMT3A, FGFR3, NPM1, FAM46C, CBFB, GATA3, MYB, CDH1, BAP1, ELF3, ZNF198, MALT1, WIF1, KDR, SFRS3, MXRA5, SS18, TAL1, RXRA, TCEA1, HEAB, THRAP3, RUNDC2A, SLC44A3, TNF, TAL2, FLJ27352, LAF4, STK19, DDX10, MSI2, NUTM2A, POU5F1, TRIP11, STAT5B, NCOA2, AZGP1, NCOA1, STAT3, NCOA4, OR52N1, CDKN2a(p14), CEP1, TFPT, SUFU, HOXA13, DB2, HOXA11, P2RY8, ECT2L, TRD@, IGH@, SMAD4, RBM10, LASP1, ROS1, KMT2D, WASF3, RBM15, PRKAR1A, KCNJ5, ATRX, EPHA2, BIRC3, HNRNPA2B1, OR4A16, NUTM2B, KLF4, MAP2K2, C15orf21, ERG, CD79A, SRGAP3, MLLT3, MITF, MN1, MLLT2, MLLT7, MLLT6, FAS, C15orf55, POU2F2, EIF2S2, MLLT4, EPS15, HERPUDI, TBC1D12, MLLT1, ALO17, CNOT3, FIP1L1, CBL, OLIG2, HOXC13, NT5C2, ABL1, ZNF521, PLAG1, TPM4, LMO1, LMO2, BLM, NTN4, SLC4A5, IRTA1, JAK3, PMS2, ATP1A1, TERT, CDH11, PTCH, DDX3X, HEYl, MORC4, TLX3, PALB2, BCR, BRCA2, MDM4, MDM2, BRD4, TFG, CSF3R, RPL10, PER1, ITPKB, PDSS2, CREB1, AF3p21, TRIM27, WRN, KIF5B, CHD8, RAB40A, GATA1, ATIC, CD1D, SETBP1, CRTC3, TNFRSF17, COL1A1, DUX4, ACVR1B, C16orf75, NIN, ZNF278, MAF, NF2, AKAP9, CCND2, MAX, MECT1, ARHGEF12, SEPT6, CBLB, FACL6, ALKBH6, CHN1, CBFA2T1, IL6ST, TCEB1, MEN1, FBXO11, HIST1H4I, RALGDS, BUB1B, FHIT, CRLF2, RASA1, TLX1, IGK@, SELP, TXNDC8, CACNA1D, GUSB, NUP214, NKX2-1, INPPL1, CBFA2T3, BCLAF1, TSC2, SDH5, CDC73, ZNF384, CDC27, OTUD7A, SIL, RANBP17, NDRG1, SMC3, FH, PAX7, CD273, HLA-B, PHOX2B, CD274, GNAS, GNAQ, PSIP1, ASPSCR1, GPHN, XIRP2, PAX8, MYOCD, FRMD7, RAP1GDS1, PAX3, AJUBA, SLC34A2, HLF, UBR5, REL, RPS2, GNA11, LHFP, TBX3, SMO, RET, PAPD5, RPS15, SS18L1, MYH11, EIF4A2, LCK, XPA, HSPCA, PPARG, CHIC2, HOXC11, H3F3B, JAK2, TFRC, ZNF620, SOX17, MTCP1, JUN, LCTL, TAF15, NONO, SRSF2, CHCHD7, MAML2, PPM1D, DAXX, H3F3A, JAK1, RIT1, CCND3, TRRAP, MED23, IGL@, SPEN, DIAPHI, CMKOR1, ZNF471, STL, POLE, MAP4K3, ING1, FOXO1A, LIFR, CHEK2, LCP1, AKT2, TPR, NFKB2, FOXL2, COL5A1, FEV, HMGA1, BCL3, HMGA2, CARS, PCSK7, ELL, GMPS, LYL1, BMPR1A, TGFBR2, SLC45A3, GRAF, HLXB9, HISTIHIE, DIS3, WWTR1, PDGFRA, PDE4DIP, ARID5B, ALDH2, STX2, SACS, ARNT, GOPC, SOS1, ITK, DICERI, KEL, CIC, RAB5EP, FVT1, PML, ADNP, FANCA, ABL2, C12orf9, BRIP1, MALAT1, FANCD2, PAFAH1B2, MUTYH, POT1, JAZF1, GNPTAB, FGFR1OP, RAD51L1, DNER, ZNF331, CD70, IKZF1, NCOR1, MLF1, MYH9, SYK, HCMOGT-1, FANCE, FANCF, FANCG, TPM3, NUP210L, INTS12, SDHC, RUNXBP2, BTG1, TTLL9, EML4, SDHB, CDK6, PMX1, PDGFRB, FOXO3A, NTRK1, CLTCL1, SH2B3, EBF1, GPC3, FGFR1, ETV6, NR4A3, SBDS, PIM1, ALPK2, PDGFB, CUL4B, YWHAE, ETV1, BCL10, PBX1, IL21R, CREB3L1, ATF1, FANCC, C2orf44, HSPCB, CANT1, PTPRC, WAS, NFIB, CREB3L2, AF1Q, NOTCH2, ABI1, SH3GL1, NBS1, OMD, SUZ12, TRA@, AF5q31, RSBN1L, BCL11B, MSH6, ERCC5, BCL11A, ERCC3, MSH2, NUMA1, KTN1, TFE3, IL2, MYCL1, LPP, HOXA9, RPL22, MSN, EVIl, BCL7A, AXIN1, NBPF1, ZNF9, MLH1, SFRS2, TRIM33, SIRT4, AXIN2, CIITA, ARHGAP35, SET, ELF4, HIP1, MSF, SOX2, FNBP1, CD74, TCL1A, RAF1, MADH4, COPEB, FLIl, CBLC, GATA2, EXT1, EXT2, MICALCL, DDIT3, D10S170, CDKN2C, MYC, GOLGA5, TRIM23, NTRK3, KLK2, SLC1A3, PRF1, ACSL3, NUP98, ELK4, CYLD, TMPRSS2, DDX6, CCNB1IP1, TTL, ZNF750, TIF1, SOCS1, PNUTL1, FOXQ1, ATP2B3, PMS1, FSTL3, PCBP1, KDM5A, ZNF145, PICALM, EWSR1, AF15Q14, BCL6, GNA13, BCL5, BCL9, ANK3, RHEB, BHD, QKI, PPP6C, CALR, PRCC, FCGR2B, BCL2, RPN1, SSX4, MDS2, TPX2, RARA, ZFHX3, TRB@, MDS1, MAFB, SLC26A3, SGK1, SDHD, CDX2, SSX1, ZRANB3, KIAA1549, SSX2, HOOK3, MTOR, SNX25, TCF1, MGA, LRIG3, PRDM16, ELKS, RHOA, ACO1, ELN, VTI1A, BRD3, MLLT10, RNF43, CDKN1A, ARID2, LCX, TFEB, WHSCILl, ETV5, ETV4, HOXD11, GAS7, ARHH, IPO7, GOT1, SMAD2, WHSC1, TNFAIP3, TCL6, HOXD13, SDC4, PAX5, MPL, MPO, SFPQ, TCF3, NACA, RECQL4, SMC1A, ERCC4, TCF12, KLHL8, DNM2, CLTC, SMARCE1, DEK, XPC, USP6, FUBP1, PCM1, TRAF7, ZRSR2, FUS, FOXP1, FLG, TOP1, MUC1, TCP11L2, COX6C, MYST4, MUC17, CAMTA1, C3orf70, CUX1, CAP2, TRAF3, MKL1, CCNE1, TSHR, AMER1, CCDC120, CHD4, and TAP1.

In some aspects, reference molecule (e.g., a reference protein) or a variant molecule (e.g., a missense variant protein) is a “pharmacogenomics target protein”, wherein the reference molecule or variant molecule is encoded by a gene selected from the group consisting of A2M, ABAT, ABCA1, ABCA12, ABCA3, ABCA8, ABCB1, ABCB11, ABCB4, ABCB5, ABCB6, ABCB9, ABCC1, ABCC10, ABCC11, ABCC2, ABCC3, ABCC4, ABCC5, ABCC6, ABCC8, ABCC9, ABCD1, ABCD2, ABCG1, ABCG2, ABCG8, ABL1, ABO, ACBD4, ACE, ACE2, ACHE, ACP5, ACSS2, ACTG1, ACY3, ACYP2, ADA, ADAM12, ADAM33, ADAMTS1, ADAMTS14, ADCK4, ADCY2, ADCY9, ADD1, ADH1A, ADH1B, ADH1C, ADH7, ADIPOQ, ADK, ADM, ADORA1, ADORA2A, ADORA2A-AS1, ADRA1A, ADRA2A, ADRA2B, ADRA2C, ADRB1, ADRB2, ADRB3, ADRBK2, AFAPIL1, AGAP1, AGBL4, AGO1, AGT, AGTR1, AGXT, AHR, AIDA, AK4, AKR1C3, AKR1C4, AKR7A2, AKT1, AKT2, ALDH1A1, ALDH1A2, ALDH2, ALDH3A1, ALDH5A1, ALG10, ALOX12, ALOX15, ALOX5, ALOX5AP, AMHR2, AMPD1, ANGPT2, ANGPTL4, ANKFN1, ANKK1, ANKRD55, ANKS1B, ANXA11, AOX1, APBB1, APEH, APLF, APOA1, APOA4, APOA5, APOB, APOBEC2, APOC1, APOC3, APOE, APOH, AQP2, AQP9, ARAP1, ARAP2, AREG, ARG1, ARHGEF10, ARHGEF4, ARID5B, ARMS2, ARNT, ARNTL, ARRB2, ARVCF, AS3MT, ASIC2, ASPH, ASS1, ATF3, ATG16L1, ATG5, ATIC, ATM, ATP2B1, ATP5E, ATP7A, ATP7B, AXIN2, B4GALT2, BACH1, BAD, BAG6, BAZ2B, BCAP31, BCHE, BCL2, BCL2L11, BCR, BDKRB1, BDKRB2, BDNF, BDNF-AS, BGLAP, BLK, BLMH, BMP5, BMP7, BRAF, BRD2, BTG4, BTRC, C10orfl07, C10orf11, C11orf30, C11orf65, C12orf40, C17orf51, C18orf21, C18orf56, Clorf167, C2, C20orf194, C3, C5, C5orf22, C8orf34, C9orf72, CA10, CA12, CACNA1A, CACNA1C, CACNA1E, CACNA1H, CACNA1S, CACNB2, CACNG2, CALU, CAMKID, CAMK2N1, CAMK4, CAP2, CAPG, CAPN10, CAPZA1, CARD16, CARTPT, CASP1, CASP3, CASP7, CASP9, CASR, CAT, CBR1, CBR3, CBS, CCDC22, CCHCR1, CCL2, CCL21, CCND1, CCNH, CCNY, CCR5, CD14, CD28, CD38, CD3EAP, CD40, CD58, CD69, CD74, CD84, CDA, CDC5L, CDCA3, CDH13, CDH4, CDK1, CDK4, CDK9, CDKAL1, CDKN2B-AS1, CELF4, CELSR2, CEP68, CEP72, CERKL, CERS6, CES1, CES1P1, CES2, CETP, CFAP44, CFB, CFH, CFI, CFLAR, CFTR, CHAT, CHIA, CHIC2, CHL1, CHRM2, CHRM3, CHRM4, CHRNA1, CHRNA3, CHRNA4, CHRNA5, CHRNA7, CHRNB1, CHRNB2, CHRNB3, CHRNB4, CHST13, CHST3, CHUK, CLASPI, CLCN6, CLMN, CLNK, CLOCK, CMPK1, CNKSR3, CNOT1, CNPY4, CNR1, CNTF, CNTN4, CNTN5, CNTNAP2, COL18A1, COL1A1, COL1A2, COL22A1, COL26A1, COLEC10, COMT, COQ2, CPA2, CPS1, CR1, CR1L, CREB1, CRH, CRHR1, CRHR2, CRP, CRTC2, CRY1, CSK, CSMD1, CSMD2, CSMD3, CSNK1E, CSPG4, CSRNP3, CSRP3, CST5, CTH, CTLA4, CTNNA2, CTNNA3, CTNNB1, CUX1, CUX2, CXCL10, CXCL12, CXCL5, CXCL8, CXCR2, CXCR4, CXXC4, CYB5A, CYB5R3, CYBA, CYCSP5, CYP11B2, CYP19A1, CYP1A1, CYP1A2, CYP1B1, CYP24A1, CYP27B1, CYP2A6, CYP2B6, CYP2B7P1, CYP2C18, CYP2C19, CYP2C8, CYP2C9, CYP2D6, CYP2E1, CYP2J2, CYP2R1, CYP39A1, CYP3A, CYP3A4, CYP3A43, CYP3A5, CYP3A7, CYP4A11, CYP4B1, CYP4F11, CYP4F2, CYP51A1, CYP7A1, DAOA, DAPK1, DBH, DCAF4, DCBLD1, DCK, DCP1B, DCTD, DDC, DDHD1, DDRGK1, DDX20, DDX53, DDX58, DEAF1, DGCR5, DGKH, DGKI, DHFR, DHODH, DIAPH3, DIO1, DIO2, DKK1, DLEU7, DLG5, DLGAP1, DMPK, DNAH12, DNAJB13, DNMT3A, DOCK4, DOK5, DOT1L, DPP4, DPYD, DPYS, DRD1, DRD2, DRD3, DRD4, DROSHA, DSCAM, DTNBP1, DUSP1, DUX1, DYNC2H1, E2F7, EBF1, ECT2L, EDN1, EGF, EGFR, EGLN3, EHF, EIF2AK4, EIF3A, EIF4E2, ENG, ENOSFI, EPAS1, EPB41, EPHA5, EPHA6, EPHA8, EPHX1, EPM2A, EPM2AIP1, EPO, ERAP1, ERBB2, ERCC1, ERCC2, ERCC3, ERCC4, ERCC5, ERCC6L2, EREG, ERICH3, ESR1, ESR2, ETS2, EXO1, F11, F12, F13A1, F2, F3, F5, F7, FAAH, FABP1, FABP2, FADS1, FAM19A5, FAM65B, FARS2, FAS, FASLG, FASTKD3, FAT1, FBXL17, FBXL19, FCAR, FCER1A, FCER1G, FCER2, FCGR2A, FCGR2B, FCGR3A, FDPS, FEN1, FGD4, FGF2, FGF5, FGFBP1, FGFBP2, FGFR2, FGFR4, FHIT, FKBP5, FLOT1, FLT1, FLT3, FLT4, FMO1, FMO2, FMO3, FMO5, FNTB, FOLH1, FOLR3, FOXC1, FOXP3, FPGS, FSHR, FSIP1, FSTL5, FTO, FYN, FZD3, FZD4, G6PD, GABRA1, GABRA3, GABRA6, GABRB1, GABRB2, GABRG2, GABRG3, GABRP, GABRQ, GAD2, GADL1, GAL, GALNT14, GALNT18, GALNT2, GALR1, GAPDHP64, GAPVD1, GATA3, GATA4, GA™, GBP6, GCG, GCKR, GCLC, GDNF, GEMIN4, GFRA2, GGCX, GGH, GHSR, GIPR, GJA1, GLCCIl, GLDC, GLP1R, GLRB, GNAS, GNB3, GNMT, GP1BA, GP6, GPR1, GPR83, GPX1, GPX3, GPX5, GRIAl, GRIA3, GRID2, GRIK1, GRIK2, GRIK3, GRIK4, GRIN1, GRIN2A, GRIN2B, GRIN3A, GRK4, GRK5, GRM3, GRM7, GSK3B, GSR, GSTA1, GSTA2, GSTA5, GSTM1, GSTM3, GSTM4, GSTP1, GSTT1, GSTZ1, H19, HAS3, HCG22, HCP5, HDAC1, HES6, HFE, HIF1A, HLA-A, HLA-B, HLA-C, HLA-DOB, HLA-DPA1, HLA-DPB1, HLA-DPB2, HLA-DQA1, HLA-DQB1, HLA-DRA, HLA-DRB1, HLA-DRB3, HLA-DRB5, HLA-E, HLA-G, HMGB1, HMGB2, HMGCR, HNF1A, HNF1B, HNF4A, HNMT, HOMER1, HOTAIR, HOTTIP, HRH1, HRH2, HRH3, HRH4, HS3ST4, HSD11B1, HSD3B1, HSPA1A, HSPA1L, HSPA5, HSPG2, HTR1A, HTR1B, HTR1D, HTR2A, HTR2C, HTR3A, HTR3B, HTR5A, HTR6, HTR7, HTRA1, HUS1, HYKK, IBA57, IDO1, IFIT1, IFNAR1, IFNB1, IFNG, IFNGR1, IFNGR2, IFNL3, IFNL4, IGF1, IGF1R, IGF2BP2, IGF2R, IGFBP3, IGFBP7, IKBKG, IKZF3, IL10, IL11, IL12A, IL12B, IL13, IL16, IL17A, IL17F, IL17RA, IL18, ILlA, IL1B, IL1RN, IL2, IL21R, IL23R, IL27, IL2RA, IL2RB, IL3, IL4, IL4R, IL6, IL6R, IL6ST, IL7R, ILKAP, IMPA2, IMPDH1, IMPDH2, INSIG2, INSR, IP6K2, IRS1, ITGA1, ITGA2, ITGA9, ITGB1, ITGB3, ITGBL1, ITIH3, ITPA, ITPKC, JAK2, KANSL1, KCNE1, KCNH2, KCNH7, KCNIP1, KCNIP4, KCNJ1, KCNJ11, KCNJ6, KCNMA1, KCNMB1, KCNQ1, KCNQ5, KCNT1, KCNT2, KDM4A, KDR, KIAA0391, KIF6, KIR2DL2, KIRREL2, KIT, KL, KLC1, KLC3, KLRC1, KLRD1, KLRK1, KRAS, KYNU, LAMB3, LARP1B, LCE3B, LCE3C, LDLR, LECT2, LEP, LEPR, LGALS3, LGR5, LIG3, LINC00251, LINC00478, LIPC, LPA, LPHN3, LPIN1, LPL, LRP1, LRP1B, LRP2, LRP5, LRRC15, LST1, LTA, LTA4H, LTB, LTC4S, LUC7L2, LYN, LYRM5, MAD1L1, MAFB, MAFK, MALAT1, MAML3, MAN1B1, MAP3K1, MAP3K5, MAP4K4, MAPK1, MAPK14, MAPT, MC1R, MC4R, MCPH1, MDGA2, MDM2, MDM4, MECP2, MED12L, MEG3, MET, METTL21A, MEX3C, MGAT4A, MGMT, MIA3, MICA, MICB, MIR1206, MIR1307, MIR133B, MIR146A, MIR2053, MIR27A, MIR300, MIR423, MIR4278, MIR449B, MIR492, MIR577, MIR595, MIR604, MIR611, MIR618, MIR7-2, MISP, MLLT3, MLN, MME, MMP1, MMP10, MMP2, MMP3, MMP9, MGB3B, MOCOS, MOV10, MPO, MPZ, MS4A2, MSH2, MSH3, MSH6, MT-RNR1, MTCL1, MTHFD1, MTHFR, MTMR12, MTOR, MTR, MTRF1L, MTRR, MTTP, MUC5B, MUTYH, MVK, MYC, MYLIP, MYOCD, N6AMT1, NALCN, NANOGP6, NAT1, NAT2, NAV2, NBAS, NBEA, NCF4, NCOA1, NCOA3, NEDD4, NEDD4L, NEFM, NELFCD, NELL1, NEUROD1, NFATC1, NFATC2, NFE2L2, NFKB1, NFKBIA, NGF, NGFR, NLGN1, NLRP3, NLRP8, NOD2, NOS1AP, NOS2, NOS3, NPAS3, NPC1L1, NPHS1, NPPA, NPPA-AS1, NQO1, NQG2, NR1D1, NR1H3, NR1I2, NR1I3, NR3C1, NR3C2, NRAS, NRG1, NRG3, NRP1, NRP2, NRXN1, NT5C1A, NT5C2, NT5C3A, NT5E, NTRK1, NTRK2, NUBPL, NUDT15, NUMA1, OAS1, OASL, OCRL, OPNISW, OPRD1, OPRK1, OPRM1, OR10AE3P, OR4D6, OR52E2, OR52J3, ORM1, ORM2, ORMDL3, OSMR, OTOS, OXT, P2RY1, P2RY12, PACSIN2, PADI4, PAPD7, PAPLN, PAPPA2, PARD3B, PARP11, PAX4, PCK1, PCSK9, PDCD1LG2, PDE4B, PDE4C, PDE4D, PDGFRA, PDGFRB, PDLIM5, PDZRN3, PEAR1, PEMT, PER2, PER3, PGLYRP4, PGR, PHACTRI, PHB2, PHTF1, PI4KA, PICALM, PICK1, PIGB, PIK3CA, PIK3R1, PITPNM2, PKLR, PLA2G4A, PLAGLI, PLCB1, PLCD3, PLCG1, PLEKHH2, PLEKHN1, PLG, PLXNB3, PMCH, POLA2, POLG, POLR3G, POMT2, PON1, PON2, POR, POU2F1, POU2F2, POU5F1, PPARA, PPARD, PPARG, PPARGC1A, PPFIA1, PPM1A, PPP1R13L, PPP1R1C, PPP2R5E, PRB2, PRCP, PRDM1, PRDM16, PRDX4, PRIMPOL, PRKAA1, PRKAA2, PRKCA, PRKCB, PRKCE, PRKCQ, PRKG1, PROC, PROCR, PROM1, PROS1, PROX1, PRRC2A, PRSS53, PSMA4, PSMB3P, PSMB4, PSMB8, PSMD14, PSORSICI, PSORS1C3, PSRC1, PTCHD1, PTEN, PTGER2, PTGER3, PTGER4, PTGES, PTGFR, PTGIR, PTGS1, PTGS2, PTH, PTH1R, PTPN22, PTPRC, PTPRD, PTPRM, PTPRN2, PYGL, RAB27A, RABEPK, RAC2, RAD18, RAD52, RAF1, RALBP1, RAPGEF5, RARG, RARS, RBFOX1, RBMS3, REEP5, REL, REN, REPS1, RET, REV1, REV3L, RFK, RGS17, RGS2, RGS4, RGS5, RHBDF2, RHOA, RICTOR, RND1, RNFT2, RORA, RPL13, RRAS2, RRM1, RRM2, RRM2B, RSBN1, RSRP1, RUNX1, RXRA, RYR1, RYR2, RYR3, SACM1L, SCAP, SCARB1, SCGB3A1, SCN10A, SCN1A, SCN2A, SCN4A, SCN5A, SCN8A, SCN9A, SCNN1B, SCNN1G, SELE, SELP, SEMA3C, SERPINA3, SERPINA6, SERPINEl, SERPINF1, SERPINGI, SETD4, SFRP5, SH2B3, SH2D5, SH3BP2, SHMT1, SIK3, SIN3A, SKIV2L, SKOR2, SLC10A2, SLC12A3, SLC12A8, SLC14A2, SLC15A1, SLC15A2, SLC16A5, SLC16A7, SLC17A3, SLC18A2, SLC19A1, SLC1A1, SLC1A2, SLC1A3, SLC1A4, SLC22A1, SLC22A11, SLC22A12, SLC22A16, SLC22A17, SLC22A2, SLC22A3, SLC22A4, SLC22A5, SLC22A6, SLC22A7, SLC22A8, SLC24A4, SLC25A13, SLC25A14, SLC25A27, SLC25A31, SLC26A9, SLC28A1, SLC28A2, SLC28A3, SLC29A1, SLC2A1, SLC2A2, SLC2A9, SLC30A8, SLC30A9, SLC31A1, SLC37A1, SLC39A14, SLC47A1, SLC47A2, SLC5A2, SLC5A7, SLC6A12, SLC6A2, SLC6A3, SLC6A4, SLC6A5, SLC6A9, SLC7A5, SLC7A8, SLCO1A2, SLCO1B1, SLCO1B3, SLCO1C1, SLCO2B1, SLCO3A1, SLCO4C1, SLCO6A1, SLIT1, SMARCADI, SMYD3, SNAP25, SNORA59B, SNORD68, SOCS3, SOD2, SOD3, SORT1, SOX10, SP1, SPARC, SPATS2L, SPECCIL, SPG7, SPIDR, SPINK5, SPP1, SPTA1, SQSTM1, SREBF1, SREBF2, SRP19, SRR, ST13, STAT3, STAT4, STAT6, STIM1, STIP1, STK39, STMN1, STMN2, STX1B, STX4, SUGCT, SULT1A1, SULT1A2, SULT1C4, SULTIEl, SULT2B1, SV2C, SYN3, SYNE3, SZRD1, T, TAAR6, TACl, TAGAP, TANC1, TANC2, TAP1, TAP2, TAPBP, TAS2R16, TBC1D1, TBC1D32, TBX21, TBXA2R, TBXAS1, TCF19, TCF7L2, TCL1A, TDP1, TDRD6, TERT, TET2, TF, TGFB1, TGFBR2, TGFBR3, TH, THBD, THRA, THRB, TIGD1, TK1, TLR2, TLR3, TLR4, TLR5, TLR7, TLR9, TMCC1, TMCO6, TMEFF2, TMEM205, TMEM258, TMEM57, TMPRSS11E, TNF, TNFAIP3, TNFRSF10A, TNFRSF11A, TNFRSF11B, TNFRSF1A, TNFRSF1B, TNFSF10, TNFSF11, TNFSF13B, TNRC6A, TNRC6B, TOLLIP, TOMM40, TOMM40L, TOP1, TOP2B, TP53, TPH1, TPH2, TPMT, TRAF1, TRAF3IP2, TRIB3, TRIM5, TRPM6, TSC1, TSPAN5, TTC6, TUBB1, TUBB2A, TXNRD2, TYMP, TYMS, UBASH3B, UBE2I, UCP2, UCP3, UGGT2, UGT1A, UGT1A1, UGT1A10, UGT1A3, UGT1A4, UGT1A5, UGT1A6, UGT1A7, UGT1A8, UGT1A9, UGT2B10, UGT2B15, UGT2B17, UGT2B4, UGT2B7, ULK3, UMPS, UPB1, USH2A, USP24, USP5, UST, VAC14, VASP, VDR, VEGFA, VKORC1, WBP2NL, WBSCR17, WDR7, WIF1, WNK1, WNT5B, WT1, WWOX, XBP1, XDH, XPA, XPC, XPO1, XPO5, XRCC1, XRCC3, XRCC4, XRCC5, YAP1, YBX1, YEATS4, ZBTB22, ZBTB4, ZCCHC6, ZFP91-CNTF, ZMAT4, ZNF100, ZNF215, ZNF423, ZNF432, ZNF652, ZNF697, ZNF804A, ZNF816, ZNRD1-AS1, and ZSCAN25.

In some aspects, the reference molecule (e.g., a reference protein) or variant molecule (e.g., a missense variant protein) is encoded by a “clinical testing gene” selected from the group consisting of LMNA, PTEN, TP53, BRCA2, MLH1, MSH2, BRCA1, MSH6, FGFR3, MECP2, CFTR, RET, PTPN11, SCN5A, MYH7, CAV3, PMS2, KRAS, APC, ATM, ARX, DMD, DES, STK11, POLG, NF1, BRAF, TSC1, CDKL5, TSC2, TTN, COL2A1, FMR1, FKTN, KCNQ1, VHL, SLC2A1, FBN1, EPCAM, HRAS, PALB2, RAF1, TNNT2, CEP290, SMAD4, MUTYH, SCN1A, SCN1B, KCNJ2, RYR2, GLA, CDH1, NRAS, FKRP, KCNH2, LDB3, CACNA1A, MYBPC3, FGFR2, UBE3A, CACNA1C, GJB2, TAZ, SDHB, TNNI3, ACTC1, GAA, TCAP, CHEK2, LAMP2, COL1A1, TTR, DSP, HBB, SDHD, SOS1, NBN, COL1A2, TGFBR2, POMT1, TPM1, FLNA, KCNE1, PCDH19, MAP2K1, CHD7, FOXG1, SDHC, TGFBR1, RYR1, MTHFR, SGCD, CDKN2A, PMP22, POMT2, FH, WT1, EMD, SCN4A, FGFR1, PLP1, PAX6, POMGNT1, TMEM43, MEN1, PKP2, SLC9A6, RHO, F5, GCK, BRIP1, TRIM32, DSG2, RAD51C, TRPV4, SCN2A, CPT2, KCNE2, GJB6, COL3A1, MAP2K2, NPHP1, DNM2, BMPR1A, PRKAG2, ACADM, OFD1, MYOT, CASQ2, HEXA, DSC2, MEF2C, HFE, CLN3, PTCH1, CRYAB, JUP, PLN, MED12, ZEB2, FHL1, ABCC8, F2, ACADVL, BAG3, ATP7A, CASR, SCN9A, BSCL2, PDHA1, SHOC2, ETFDH, KCNQ2, HADHA, TNNC1, PRRT2, TPP1, ANO5, COL5A1, ETFB, MPZ, ETFA, ACTA1, PPT1, CASK, STXBP1, ABCD1, KCNJ11, ATRX, GNAS, ABCA4, DYSF, ABCC9, TCF4, BLM, SLC22A5, SDHA, MYH6, HCN4, ATP7B, PLA2G6, FANCC, MYL2, CBS, ANK2, KCNE3, MYL3, CLN5, DCX, PANK2, ALDH7A1, NKX2-5, GBA, TIMM8A, PNKP, ACTA2, WFS1, MFN2, FOLR1, JAG1, SMN1, SMARCB1, L1CAM, GPC3, KIT, NSD1, OPA1, DHCR7, NF2, SGCA, MITF, CLRN1, TPM2, SPRED1, MKS1, NIPBL, AGL, OTC, RB1, CSRP3, GLB1, TMEM67, CLN6, HNF1B, SMC1A, SCN4B, CACNB2, ACVRL1, DLD, CBL, FXN, ARSA, PSEN1, COL6A3, LAMA2, SMAD3, ENG, PRPS1, ACTN2, TWNK, CAPN3, GDAP1, COL5A2, EYA1, PCDH15, GCH1, SURFi, SGCB, SCN3B, TMEM216, PITX2, COL6A1, PEX1, MYH11, VCL, NOTCH3, LARGE1, SLC26A4, CLN8, BTD, GAMT, USH2A, MYH9, AR, NPC1, TERT, GABRG2, GCDH, HNF1A, FLNC, IDS, COL6A2, BBS1, RPGR, FLCN, GNE, RPGRIP1L, MEFV, CALM1, CDKN1C, MFSD8, PRPH2, SMPD1, OPHN1, CNTNAP2, BCKDHB, PLOD1, PLEC, CREBBP, SDHAF2, ARHGEF9, AKAP9, RAD51D, NEB, OPA3, MBD5, NPC2, MYO7A, CTSD, VPS13B, GALC, KCNJ5, PAFAHIBI, PYGM, GRN, ASPA, CDK4, PEX7, MET, FBN2, CC2D2A, GARS, NRXN1, PIK3CA, COL11A2, HTT, SLC26A2, SETX, NEXN, TGFB3, SELENON, KCNJ10, CPT1A, HPRT1, ELN, UGT1A1, WAS, OCRL, KCND3, MUT, VCP, HADHB, GPD1L, KCNQ3, SUCLA2, SCO2, FTL, EGR2, PMM2, ALPL, SNTA1, BBS2, G6PC, HADH, PKD2, PKHD1, COQ2, MMACHC, GJB1, BEST1, SGCG, BCKDHA, LDLR, NPHP3, SLC25A20, ACADS, DYNCIHI, KCTD7, MAPT, FIG4, TREX1, MMAB, PQBP1, GRIN2A, COL4A5, MMAA, MKKS, RPE65, GBE1, NDP, HSD17B10, GATA1, APOB, TTC8, SPG7, PDX1, GABRA1, APTX, IKBKAP, NEFL, PEX6, COL11A1, TBC1D24, TGFB2, CRX, APOE, GUCY2D, PHOX2B, ISPD, ATP1A2, ATP13A2, ATL1, SYNE1, ATXN2, SLC6A8, ALMS1, HNF4A, AHIl, ACAD9, PRKAR1A, SNRPN, COL4A1, NOTCH1, SLC25A22, GLDC, ADGRV1, GALT, PEX26, TRDN, PHF6, PNPO, KCNT1, MTM1, COX15, SLC4A1, RRM2B, PRSS1, TPM3, BBS10, BAP1, BCS1L, CDH23, MRE11, PCCA, TBX5, MPL, PAH, SPTAN1, SCN8A, AMT, ASS1, PSEN2, CACNA1S, USH1C, FANCA, CYP21A2, FGD1, PEX12, SLC2A10, WDR62, FAH, GLI3, RUNX1, ANKRD1, GNPTAB, SLC25A4, SERPINA1, RELN, BARD1, RAPSN, DKC1, CSTB, SGCE, F8, KCNJ8, MYPN, MVK, PEX10, REEP1, CRB1, CHRNA1, RBM20, PCCB, BCOR, NLRP3, HBA1, EPM2A, SKI, GATA2, MYLK, FANCB, TYR, ABCB4, C12orf65, PEX2, LRP5, TTC21B, SLC25A13, HSPB1, HSPB8, MPV17, SPAST, SLC37A4, IQCB1, IDUA, EYA4, KCNA1, PGK1, CYP1B1, WHRN, SMARCA4, TERC, ADSL, DMPK, ATXN1, ATP6AP2, SYNGAP1, RDH12, TARDBP, KMT2D, PRKN, NPHP4, TK2, NHLRC1, GJA1, SUCLG1, GATA4, NDUFA1, COL4A3, ATXN3, VWF, TH, DBT, KIF1A, MMADHC, MID1, PKD1, AP3B1, CHRNA4, DNAJB6, APP, SHH, FA2H, CHRNB2, EDN3, SLC16A2, ELANE, FUS, INS, RPS6KA3, INVS, MYOZ2, TNNT1, ALK, TMEM70, CACNB4, JAK2, CNGB3, SPINKI, AGXT, PAX3, MCOLN1, PEX5, ASPM, DGUOK, IGHMBP2, CFH, SOD1, TUBA1A, DOLK, PROM1, SYN1, HMGCL, KDM5C, RAB39B, DNAJC5, AUH, SHOX, ATXN7, CENPJ, SRPX2, SOX10, CYP2D6, DCTN1, TBX1, ALDOB, ARL6, BBS12, COQ8A, TWISTI, RECQL4, OTX2, PC, DPAGT1, TP63, GP1BA, ARG1, POLD1, SACS, AKT1, PEX3, SMC3, OCA2, CYP2C19, RMRP, IL2RG, DNAH5, SPG11, NDRG1, COL4A4, FOXC1, BMPR2, MCCC2, MAX, F9, ERCC6, C9orf72, TYMP, RAI1, AIPL1, MCCC1, SLC25A19, COL9A1, BTK, P3H1, PDSS2, PCNT, NOTCH2, ATP8B1, ATP1A3, ETHEl, HEXB, SLC25A15, CP, COL9A2, CHRNA2, CHRNE, CUL4B, DOK7, CHRND, GUSB, SLC19A3, IVD, SH3TC2, EFHC1, IMPDH1, CRTAP, CYP27A1, HSPD1, SOX2, SDCCAG8, CYP2C9, ALS2, RPS19, GOSR2, RARS2, GFAP, PEX14, CYP11B1, GMPPB, BBS4, SGSH, GJC2, GLUD1, GATM, TMEM127, RPGRIP1, PDGFRA, LGI1, MT-ATP6, ADAMTS13, BBS5, WDR45, MTMR2, GATA6, BBS7, LITAF, POLG2, ABCB11, PRX, ALG2, ABCC6, RNASEH2B, FANCG, ADA, SIL1, RP2, RASA1, NTRK1, TNFRSF1A, SCNN1B, CHAT, USH1G, FLNB, DNAI1, CFL2, OPTN, NDUFS4, ARL13B, BBS9, TOR1A, LRPPRC, ATPAF2, SAMHDI, TSEN54, NPHS2, TSFM, HBA2, GALNS, FKBP14, CHST14, FOXRED1, TRPM4, NHS, RNASEH2A, RNASEH2C, ADGRG1, MT-RNR1, AGK, CEP152, ASL, SNCA, GRIN2B, DTNA, SIX1, CPS1, KIF7, AIFM1, PDHX, NAGLU, MT-TL1, NSDHL, HDAC8, HGSNAT, LRRK2, SBF2, RAB7A, SCNN1G, LRAT, DARS2, KIF5A, RIT1, PCSK9, GFM1, PINK1, NPHS1, ARSB, NDUFS7, POLE, PFKM, SCN2B, IDH2, FBLN5, INPP5E, PDSS1, GABRD, ATP6VOA2, PRICKLE1, ACAT1, SOX9, CACNA2D1, G6PD, SPG20, SCARB2, NLGN3, ANOS1, NLGN4X, GABRB3, HAX1, AFG3L2, GJB3, TINF2, KRIT1, GPR143, CDC73, EDNRB, MLYCD, AARS2, JAK3, SDHAF1, JPH2, NDUFV1, PEX13, PLCB1, ABHD12, PEX16, IRF6, SUMF1, BSND, DAG1, HLCS, ATR, EGFR, AFF2, EZH2, PEX19, ABCA3, PAK3, NDUFS1, PHYH, PRKCG, TMPO, TULP1, COMP, MPI, MYLK2, HESX1, YARS, BIN1, DPM3, LYST, AARS, SIX3, ACTG1, C19orf12, PDHB COQ9, MLC1, NODAL, DPYD, CHM, DPM1, LIPA, SFTPC, DLAT, VRK1, TUBB2B, ATP6V1B1, HSD17B4, CERKL, EP300, SLC12A3, GATA3, FANCE, FGD4, CFI, SCN10A, COLQ, COX6B1, FKBP10, EXT1, ADAMTS2, SBDS, CD46, TGIF1, SALL1, ERCC4, KIF1B, SLC17A5, WNK1, KCNA5, ARFGEF2, FANCF, ELOVL4, SALL4, CYP7B1, KARS, GRIA3, ALDH5A1, SPR, CLCN1, HCCS, GNS, EIF2AK3, PUS1, PDE6B, PLOD2, PAX2, DHDDS, WDR19, ALG6, PPARG, VAPB, CHD2, RP1, PSAP, WRN, LMBRD1, INSR, CEBPA, LPIN1, SMS, MT-TK, PARK7, SUFU, UMOD, PRNP, AGA, RAD50, FUCA1, SLC39A13, NDUFA2, ISCU, MT-TS1, SEMA4A, FOXP3, TACO1, LIG4, AIRE, SRY, KBTBD13, EIF2B5, MT-ND1, IKBKG, DICERI, TRMU, MUSK, SLC25A3, OTOF, POMK, TBP, RAG2, UPF3B, EDA, RLBP1, RAB3GAP1, LAMB2, CEP41, RAD21, KDM6A, MCPH1, CABP4, SPATA7, MTRR, LAMA4, EFEMP2, NDUFS8, GALK1, SAG, LCA5, NR2E3, EXT2, GCSH, PPIB, PORCN, EHMT1, CTNNB1, CTNS, TFR2, C3, HCN1, EIF2B1, SLX4, POU3F4, WDPCP, INF2, LIAS, CHRNB1, ACTB, AP1S2, PHEX, SPTB, NEUROD1, RS1, NPPA, SOX3, FGF23, MAN2B1, DNAH11, ERCC2, DGKE, CCM2, NDUFAF2, EVC, RAG1, HPS1, NDUFS3, NDUFS2, ZIC2, FGF8, LPL, FASTKD2, TCTN2, CACNA1D, HPS4, CACNA1F, CLCN5, GJA5, SYP, GP1BB, FANCL, ACSL4, IDH1, CLCNKB, CISD2, ROR2, NEU1, GATADI, MYH3, NDE1, PRPF31, ABCG5, NKX2-1, PGM1, TMEM237, FBP1, CDK5RAP2, NDUFAF5, ZFYVE26, DPM2, PHKA1, MT-ND6, STIL, TUBB3, BICD2, IQSEC2, SPTA1, ITGA7, QDPR, TJP2, PTS, EIF2B3, NOD2, GLRA1, CSF1R, PRF1, ATN1, PAX4, GPSM2, CHMP2B, CFB, EYS, FANCI, ST3GAL3, AGPAT2, PDP1, IL7R, HK1, PNPLA2, RAB27A, DCLRE1C, MC4R, GYS2, B9D1, SCNN1A, ANG, ENPP1, PRPF8, SFTPB, FANCM, AXIN2, LMX1B, NHEJ1, SYNE2, TTC19, PROP1, MAGT1, COL7A1, FANCD2, FSCN2, NDUFAF1, MT-ND4, KCNJ1, COL12A1, CNGA3, STAT3, TYRP1, NDUFS6, GUCAIB, SLC2A2, SIX5, ADAR, SLC33A1, CCDC39, AMACR, GAN, HFE2, B3GLCT, EFNB1, UQCRB, SLC12A6, FGA, HPS3, XRCC2, MTR, C8orf37, ACTN4, EVC2, THAP1, TRPS1, IDH3B, RUNX2, LAMB3, SH2D1A, GDI1, TMC1, DNMT1, PDCD10, MRPS22, LAMA3, TOPORS, CHKB, MTPAP, CYP17A1, POMGNT2, SLC12A1, ZIC3, GLI2, RD3, ALAS2, RPL35A, CNGB1, LDLRAP1, DEPDC5, THBD, DYRK1A, SLC19A2, DNAI2, PGAM2, PNKD, ASAH1, WDR35, VKORC1, DOCK8, PHGDH, SLC45A2, GP9, CCDC78, SPTLC1, IL1RAPL1, SLC35C1, UBE2A, NROB1, CAVIN1, ACOX1, AGRN, CA4, COL9A3, CNGA1, LAMC2, DTNBP1, EIF2B2, TTPA, FLVCR1, MYH14, ERBB2, ITGB3, VLDLR, WASHC5, NDUFA11, C2orf71, PTCHD1, NRL, ALDH4A1, RSPH9, ATP5E, GK, CTDP1, ABL1, TCTN1, ANK1, CTSA, SLC40A1, AKT3, B4GAT1, ZMPSTE24, MERTK, EIF2B4, ERCC8, NUBPL, PPOX, PDLIM3, PNPLA6, TNXB, PRKG1, FOXH1, COG7, RPL11, GPHN, ABCG8, PDE6C, B4GALT7, G6PC3, GNA11, CLCN2, NME8, KCNJ13, HEPACAM, SLCO1B1, UQCRQ, NDUFAF4, TMEM138, MT-ND5, NDUFAF3, HMBS, NHP2, IFITM5, MBTPS2, SMN2, PDE6A, VSX2, MYO6, CPOX, ALG13, CCDC40, ALDH3A2, NIPA1, TSHR, ZNF423, SQSTM1, MOCS2, L2HGDH, SCO1, TUBB4A, TCOF1, MOCS1, MTO1, CIB2, HINT1, KIAA2022, ERCC3, PITX3, PRPF3, DNM1L, TCTN3, FHL2, CA2, GRHPR, PLEKHG5, CDON, KLHL40, TSEN2, SLC1A3, RGR, NEBL, C5orf42, HPS6, GFI1, MYCN, LZTR1, BRWD3, TSEN34, F11, SNRNP200, GNAT2, ALG1, TMEM126A, SP7, KLHL7, TUFM, DLG3, DNAAF2, DNAAF1, VPS13A, NOP10, TMEM5, MCEE, STXBP2, MED25, SHANK3, SLC3A1, TECTA, COX10, CHRNG, RDH5, CDHR1, PHF8, RPL5, MAOA, GFPT1, RAB3GAP2, CALM2, NAGS, POLR1C, HSD3B2, AMPD1, BUB1B, NEK8, TUBA8, B3GALNT2, FLT3, MATR3, KRT5, GDF6, GREM1, AVPR2, DNAL1, ZDHHC9, CTC1, ALDOA, NR5A1, CYBB, FTSJ1, BLOC1S3, EBP, DCAF17, SPG21, ACAD8, ABCB7, F12, GLRB, GLIS2, EXOSC3, HUWEl, BMP4, TMIE, GNPTG, RPS26, ITGA2B, LRSAM1, SLC6A3, ALDH18A1, SERPINC1, KLF11, F7, RPS10, WNT10A, NFIX, MGAT2, ACSF3, RBBP8, CFHR5, COQ6, UBQLN2, CDKN1B, SUOX, FAM126A, COG8, NDUFA10, SMARCE1, ALG8, GSS, EPB42, RPL10, DNAJC19, NAA10, KCNMA1, RPS24, STX11, ALG3, XK, MFRP, TMPRSS3, TSPAN7, SERPINHI, IMPG2, ALG12, SERPINEl, SLC16A1, TCIRG1, STIM1, ETV6, CLCN7, GDF2, SLC35A1, FAM161A, ARID1B, TMEM231, SLC35A2, NGF, COX4I2, POU1F1, GLIS3, TAF1, PNP, POMC, KIF1BP, BLK, YARS2, TCN2, UNC13D, HAMP, HOGA1, ACADSB, B4GALT1, MANBA, KAT6B, RSPH4A, ACE, EDAR, WWOX, FARS2, GNAQ, GNPAT, ANKH, ENO3, FRAS1, RANGRF, GALE, TREM2, CD3D, LEP, TFG, IER3IP1, DYNC2H1, NPM1, KMT2A, CD40LG, PYGL, MT-CYB, DFNB59, MRPS16, RTN2, KCNE5, MATN3, TAT, NDUFV2, CDAN1, STS, CAV1, B3GALT6, CTSK, CALR3, KCNV2, AP4M1, SERPINGI, GYS1, HPS5, ST3GAL5, SLC6A5, ARID1A, PRKRA, COG1, COL4A2, EFEMP1, PIK3R2, MTFMT, SEPT9, FOXP1, NDUFAF6, ROM1, KRT14, SLC25A12, SEC23B, TNNI2, CD3E, HPD, PHKB, AIP, FZD4, XPNPEP3, CEP164, ITGB4, SLMAP, PABPN1, TBCE, GHR, NOG, CACNA2D4, ALG9, FOXL2, TYROBP, THRB, AP4E1, BDNF, AKT2, DSPP, MPDU1, EDARADD, TPMT, SPTBN2, BLOC1S6, FGF14, CTSF, PRCD, SRD5A3, PRPF6, TRAPPC11, PHKA2, COCH, AGPS, EARS2, FOXE3, IGBP1, RBP3, PKLR, PIGA, MAT1A, SPTLC2, CEP63, FBXO7, SETBP1, OTOA, RTEL1, PTF1A, LEPR, SMARCAL1, SCP2, PCBD1, DMP1, MOGS, CNTN1, TNPO3, POLR3A, SLC46A1, FOXI1, MYO15A, KCNQ4, MYOC, PYCR1, APOA5, GRHL2, POR, AICDA, KISSIR, PRDM16, ARSE, LHFPL5, PDE6G, HARS, SNAI2, VCAN, SMPX, CSF3R, COL17A1, LOXHD1, MTTP, SERPINF1, PROKR2, GNRHR, D2HGDH, B9D2, ZAP70, AP5Z1, CTNNA3, CSF2RA, SLC34A3, ZNF513, TNFRSF11A, CTRC, RP9, HSPG2, KANSL1, RPS7, TRIOBP, CEL, SHROOM4, SLC7A7, RFT1, ADAMTSL4, ABCA12, ABAT, LPIN2, ERCC5, HGF, PROC, LHX4, ROGDI, ABCA1, DIABLO, ESCO2, PRDM5, PHKG2, FREM1, PRODH, DIS3L2, RDX, WRAP53, MC1R, ACVR1, ZNF711, IFT80, ACVR2B, EFTUD2, LTBP2, MEGF10, RAB18, CLDN14, FLT4, CCT5, SRCAP, ESRRB, PDZD7, NEK1, NR3C2, TBX20, DNAJB2, FAS, ATXN10, CFHR1, GDF5, PSTPIP1, ARHGEF6, TDP1, GUCA1A, OXCT1, PPP2R2B, AQP2, TRPC6, MARVELD2, FECH, OAT, PEX11B, PRICKLE2, APOC2, PDGFRB, CACNA1H, LHCGR, SARS2, LRTOMT, COL10A1, XIAP, UNG, MGME1, SLC26A5, CYBA, PITPNM3, PTH1R, TIMP3, DRD2, PDE6H, ALX4, TXNRD2, OBSL1, ORC1, GH1, CSPP1, LEFTY2, CCDC50, ABCD4, DIAPHI, CDH3, CHCHD10, PAX8, GDNF, MT-CO1, HARS2, HTRA1, BMP1, MSRB3, ZDHHC15, CAVIN4, AP4S1, CFHR3, ACADL, NDUFA9, MSX1, MYO3A, CYP11B2, CTF1, MAK, AP4B1, IFT122, ABHD5, MARS, A2ML1, CHST3, CYLD, GDF1, XPA, MT-TH, TPRN, MT-TQ, POU4F3, XPC, GRIN1, GIPC3, CYP27B1, POLR1D, LHX3, TGFB1, TOR1AIP1, CNBP, GM2A, DDHD2, TRPM1, BCKDK, DNAAF3, HSD11B2, ADAM9, CLCNKA, NDUFB3, LAS1L, MAGI2, ANKRD11, NMNAT1, ZFYVE27, DNMT3A, PROK2, SMARCA2, GFER, POLR3B, NDUFA12, PLCE1, STRA6, EMX2, HMGCS2, ASCL1, COMT, PROS1, KCNC3, ILK, FGB, C10orf11, ILDR1, ANKRD26, GRXCR1, SZT2, HNRNPDL, KIF11, FGG, DDC, TTBK2, FREM2, ZNF469, TUSC3, TFAP2A, DLL3, CLIC2, GDF3, MT-TS2, CYP3A5, AHCY, LDHA, SLC52A3, PRKCSH, ACYl, ACO2, KCNK3, AMER1, WNT1, MARS2, NYX, VPS35, UROS, COG6, REN, AVP, MTOR, TBX3, RBM10, PFN1, TPO, MYBPC1, SERPINB6, PTPRC, H19, ABCB6, WNT7A, MYOSA, CCDC88C, ATP6VOA4, OSTM1, SRD5A2, CDT1, DFNA5, ESPN, MYF6, USB1, DDOST, CRYM, APOA1, ATXN8OS, AGTR2, SLC17A8, MSX2, DST, LTBP4, KLHL3, AAAS, RFX6, LBR, CYP3A4, F13A1, RAX2, RAC2, PREPL, ERLIN2, ANK3, NFU1, LRP4, TNFRSF13B, TNFSF11, SNAP29, LAMC3, RBM8A, ORC6, GRM6, COGS, ORC4, PDYN, CRELD1, SLC5A7, ITGA3, SPINK5, WNT4, ENAM, C1QTNF5, PDK3, HTRA2, GNB4, WNK4, COG4, MT-TI, HSPB3, MT-TL2, HCFC1, POT1, ICOS, SIGMAR1, ATP2A1, GNAT1, SOS2, CTSC, FOXP2, TMEM165, CXCR4, SH3BP2, TACR3, CFC1, ABCC2, DNAJC6, DHODH, CPA6, AK2, HOXD13, VPS45, PLOD3, KRT1, MT-ATP8, DNAAF5, TGM1, TSPAN12, IFT172, CD2AP, MRPL3, LIFR, RIMS1, CNNM4, CDC6, F10, FOXC2, STAT5B, PIK3R1, ORAI1, ZNF81, ZFP57, CYP24A1, GLE1, COL18A1, TIA1, RPL26, GNAO1, LCAT, VDR, ANO10, TNNT3, LZTFL1, COL4A6, and SHANK2.

### IV. Computer Systems and Readable Devices

The methods described herein, due to the complexity of the calculations involved, e.g., conformational dynamics generation (e.g., MD simulations), dimensionality reduction, ML model training, or any combination thereof, can be implemented with the use of a computer system. Accordingly, in some aspects, the present disclosure provides a system comprising (a) a memory; and, (b) at least one processor coupled to the memory and configured for (i) generating conformational dynamics of a reference molecule (e.g., using MD); (ii) processing the simulated conformational dynamics of the reference molecule to generate a low-dimensional representation of the conformational landscape of the reference molecule; (iii) extracting a conformational state from the low-dimensional conformational landscape of the reference molecule to seed simulations of a variant molecule (e.g., a variant protein); (iv) generating a variant protein or set thereof by introducing at least one missense mutation in a simulated conformation of the reference molecule in the extracted conformational state; (v) generating conformational dynamics of the variant molecule or set thereof (e.g. using MD); (vi) processing the conformational dynamics of the variant molecule or set thereof to generate a low-dimensional representation of the conformational landscape of the variant molecule or set thereof; and (vii) training a machine-learning predictive model using the low-dimensional representation of the conformational landscape reference molecule and variant molecule using clinical data as training labels, wherein the predictive model classifies the variant (e.g., a missense mutation) effects in the reference molecule.

The present disclosure also provides a tangible computer readable device having instruction stored thereon that, when executed by at least one computing device, causes the at least one computing device to perform operations comprising (a) generating conformational dynamics (e.g., using MD) of a reference molecule (e.g., a reference protein); (b) processing the conformational dynamics of the reference molecule to generate a low-dimensional representation of the conformational landscape of the reference molecule; (c) extracting a state from the low-dimensional conformational landscape of the reference molecule to seed simulations of a variant molecule; (d) generating a variant molecule or set thereof by introducing at least one variant (e.g., a missense mutation) in a simulated conformation of the reference molecule in the extracted state; (e) generating conformational dynamics of the variant molecule or set thereof (e.g. using MD); (f) processing the conformational dynamics of the variant molecule or set thereof to generate a low-dimensional representation of the conformational landscape of the variant molecule or set thereof; and (g) training a machine-learning predictive model using the low-dimensional representation of the conformational landscape of the reference molecule and variant molecule using clinical data as training labels, wherein the predictive model classifies the variant (e.g., a protein missense mutation) effects in the reference molecule.

The present disclosure provides a computer system for implementing the methods described herein, e.g., ML predictive models for diagnosing and/or predicting the phenotypic impact of a variant (e.g., a missense variant) and/or the steps to generate such ML predictive models. A computer system is an apparatus or device including one or more processors, a memory, input/out interface, and a network interface. In some aspects, the computer system comprises hardware elements that are electrically coupled via bus, including a processor, input device, output device, storage device, computer-readable storage media reader, communications system, processing acceleration (e.g., DSP or special-purpose processors), and memory.

The computer-readable storage media reader can be further coupled to computer-readable storage media, the combination comprehensively representing remote, local, fixed and/or removable storage devices plus storage media, memory, etc. for temporarily and/or more permanently containing computer-readable information, which can include storage device, memory and/or any other such accessible system resource. In some aspects, the memory is communicatively coupled with one or more processors. The one or more processors are configured to execute computer program instructions for implementing the methods described herein. The computer program instructions are stored on and/or provided by the memory. Examples of computers include personal computers, laptops, computer clusters, supercomputers, and any other kind of computer.

Memory is an example of a computer-readable medium. It may include volatile storage devices in computer-readable media, random access memory (RAM), and/or non-volatile memory, such as read-only memory (ROM) or flash memory (flash RAM). Computer-readable media include permanent, non-permanent, removable, and non-removable media. Computer-readable commands, data structures, program modules are examples of information stored on the computer-readable media. Examples of computer storage media include phase-change memory (PRAM), static random access memory (SRAM), dynamic random access memory (DRAM), other types of random access memory (RAM), read-only memory (ROM), flash memory, compact disk read-only memory (CD-ROM), digit multifunction disc (DVD), and magnetic tape. Computer-readable media does not include temporary or transitory computer-readable media, such as modulated data signals and carrier waves.

The computer processors include a plurality of components including, multicore CPUs, multi-CPU computers and computer clusters, many-core CPUs, general purpose graphical processor units (gpGPUs), and other computer processors.

In embodiments, the methods described herein, e.g., ML predictive models for diagnosing and/or predicting the phenotypic impact of a variant (e.g., missense variant) and/or the steps to generate such ML predictive models, are performed using computer processors of sufficient computational power to perform the simulations and analysis. Currently molecular simulation and machine learning are done on parallel architectures such as multi-core CPU, multi-CPU systems, and general-purpose GPUs. In embodiments, the computer system for performing the methods described herein, e.g., ML predictive models for diagnosing and/or predicting the phenotypic impact of a variant (e.g., a missense variant) and/or the steps to generate such ML predictive models, includes memory sufficient for the calculations and storage such as disks, disk arrays, solid state drives (SSDs) with speed sufficient for the simulations and analysis.

A single architecture might be utilized to implement one or more servers that can be further configured in accordance with currently desirable protocols, protocol variations, extensions, etc. However, it will be apparent to those skilled in the art that aspects may well be utilized in accordance with more specific application requirements. Customized hardware might also be utilized and/or particular elements might be implemented in hardware, software or both. Further, while connection to other computing devices such as network input/output devices (not shown) may be employed, it is to be understood that wired, wireless, modem, and/or other connection or connections to other computing devices might also be utilized.

In one aspect, the system further comprises one or more devices for providing input data to the one or more processors. The system further comprises a memory for storing a dataset of ranked data elements. In another aspect, the device for providing input data comprises a detector for detecting the characteristic of the data element, e.g., such as a fluorescent plate reader, mass spectrometer, gene chip reader, or gene sequencer.

The system additionally may comprise a database management system. User requests or queries can be formatted in an appropriate language understood by the database management system that processes the query to extract the relevant information from the database of training sets. The system may be connectable to a network to which a network server and one or more clients are connected. The network may be a local area network (LAN) or a wide area network (WAN), as is known in the art. Preferably, the server includes the hardware necessary for running computer program products (e.g., software) to access database data for processing user requests. The system can be in communication with an input device for providing data regarding data elements to the system (e.g., expression values).

Some aspects described herein can be implemented to include a computer program product. A computer program product may include a computer readable medium having computer readable program code embodied in the medium for causing an application program to execute on a computer with a database. As used herein, a “computer program product” refers to an organized set of instructions in the form of natural or programming language statements that are contained on a physical media of any nature (e.g., written, electronic, magnetic, optical or otherwise) and that may be used with a computer or other automated data processing system. Such programming language statements, when executed by a computer or data processing system, cause the computer or data processing system to act in accordance with the particular content of the statements.

Computer program products include without limitation: programs in source and object code and/or test or data libraries embedded in a computer readable medium. Furthermore, the computer program product that enables a computer system or data processing equipment device to act in pre-selected ways may be provided in a number of forms, including, but not limited to, original source code, assembly code, object code, machine language, encrypted or compressed versions of the foregoing and any equivalents. In one aspect, a computer program product is provided to implement the treatment, diagnostic, prognostic, or monitoring methods disclosed herein, for example, to determine whether to administer a certain therapy based on the classification of a variant protein according to the ML predictive models disclosed herein.

In some aspects, the computer program product includes a computer readable medium embodying program code executable by a processor of a computing device or system, the program code that executes at least one of the computer implemented methods disclosed herein or an ML predictive model generated according to the methods disclosed herein. In some aspects, the computer readable medium can comprise libraries of precomputed MD structures, precomputed models, precomputed variant pathogenicity scores, or any combination thereof.

While various aspects have been described as methods or apparatuses, it should be understood that aspects can be implemented through code coupled with a computer, e.g., code resident on a computer or accessible by the computer. For example, software and databases could be utilized to implement many of the methods discussed above. Thus, in addition to aspects accomplished by hardware, it is also noted that these aspects can be accomplished using an article of manufacture comprised of a computer usable medium having a computer readable program code embodied therein, which causes the enablement of the functions disclosed in this description. Therefore, it is desired that aspects also be considered protected by this patent in their program code means as well.

Furthermore, some aspects can be code stored in a computer-readable memory of virtually any kind including, without limitation, RAM, ROM, magnetic media, optical media, or magneto-optical media. Even more generally, some aspects could be implemented in software, or in hardware, or any combination thereof including, but not limited to, software running on a general purpose processor, microcode, PLAs, or ASICs.

It is also envisioned that some aspects could be accomplished as computer signals embodied in a carrier wave, as well as signals (e.g., electrical and optical) propagated through a transmission medium. Thus, the various types of information discussed above could be formatted in a structure, such as a data structure, and transmitted as an electrical signal through a transmission medium or stored on a computer readable medium.

### V. Companion Diagnostics

The methods disclosed herein or the predictive models generated as described herein can be provided as a companion diagnostic, for example available via a web server, to inform the clinician or patient about potential treatment choices. The methods disclosed herein can comprise collecting or otherwise obtaining a biological sample and performing an analytical method, e.g., apply a ML predictive model disclosed herein to classify a variant (e.g., a mutation in a variant protein), for example, as pathogenic, and based on the assignment provide a suitable treatment for administration to the patient.

### VI. Accuracy and Clinical Performance Metrics of ML Predictive Model

In general, predictive models developed for medical diagnosis should be validated and their accuracy should be determined to be trustworthy by doctors. Multiple performance measures can be used to validate a ML predictive model, e.g., like accuracy, RO curve, kappa statistics, MAE, RMSE, etc.

The accuracy of a ML predictive model for diagnosing and/or predicting the phenotypic impact of a variant (e.g., a missense variant) disclosed herein can be determined by the 95% confidence interval (CI). Generally, a predictive model is considered to have good accuracy if the 95% CI does not overlap 1. In some aspects, the 95% CI of a predictive model is at least about 1.08, at least about 1.10, at least about 1.12, at least about 1.14, at least about 1.15, at least about 1.16, at least about 1.17, at least about 1.18, at least about 1.19, at least about 1.20, at least about 1.21, at least about 1.22, at least about 1.23, at least about 1.24, at least about 1.25, at least about 1.26, at least about 1.27, at least about 1.28, at least about 1.29, at least about 1.30, at least about 1.31, at least about 1.32, at least about 1.33, at least about 1.34, or at least about 1.35 or more. The 95% CI of a predictive model may be at least about 1.14, at least about 1.15, at least about 1.16, at least about 1.20, at least about 1.21, at least about 1.26, or at least about 1.28. The 95% CI of a predictive model may be less than about 1.75, less than about 1.74, less than about 1.73, less than about 1.72, less than about 1.71, less than about 1.70, less than about 1.69, less than about 1.68, less than about 1.67, less than about 1.66, less than about 1.65, less than about 1.64, less than about 1.63, less than about 1.62, less than about 1.61, less than about 1.60, less than about 1.59, less than about 1.58, less than about 1.57, less than about 1.56, less than about 1.55, less than about 1.54, less than about 1.53, less than about 1.52, less than about 1.51, less than about 1.50 or less. The 95% CI of a predictive model may be less than about 1.61, less than about 1.60, less than about 1.59, less than about 1.58, less than about 1.56, 1.55, or 1.53. The 95% CI of a predictive model may be between about 1.10 to 1.70, between about 1.12 to about 1.68, between about 1.14 to about 1.62, between about 1.15 to about 1.61, between about 1.15 to about 1.59, between about 1.16 to about 1.160, between about 1.19 to about 1.55, between about 1.20 to about 1.54, between about 1.21 to about 1.53, between about 1.26 to about 1.63, between about 1.27 to about 1.61, or between about 1.28 to about 1.60.

In some aspects, the accuracy of a ML predictive model for diagnosing and/or predicting the phenotypic impact of a variant (e.g., a missense variant) disclosed herein is dependent on the difference in range of the 95% CI (e.g., difference in the high value and low value of the 95% CI interval). Generally, ML predictive models with large differences in the range of the 95% CI interval have greater variability and are considered less accurate than ML predictive models with small differences in the range of the 95% CI intervals. In some aspects, a ML predictive model is considered more accurate if the difference in the range of the 95% CI is less than about 0.60, less than about 0.55, less than about 0.50, less than about 0.49, less than about 0.48, less than about 0.47, less than about 0.46, less than about 0.45, less than about 0.44, less than about 0.43, less than about 0.42, less than about 0.41, less than about 0.40, less than about 0.39, less than about 0.38, less than about 0.37, less than about 0.36, less than about 0.35, less than about 0.34, less than about 0.33, less than about 0.32, less than about 0.31, less than about 0.30, less than about 0.29, less than about 0.28, less than about 0.27, less than about 0.26, less than about 0.25 or less. The difference in the range of the 95% CI of a ML predictive model may be less than about 0.48, less than about 0.45, less than about 0.44, less than about 0.42, less than about 0.40, less than about 0.37, less than about 0.35, less than about 0.33, or less than about 0.32. In some aspects, the difference in the range of the 95% CI for ML predictive model is between about 0.25 to about 0.50, between about 0.27 to about 0.47, or between about 0.30 to about 0.45. In some aspects, the sensitivity of an ML predictive model for diagnosing and/or predicting the phenotypic impact of a variant (e.g., a missense variant) disclosed herein is at least about 60%. In some aspects, the sensitivity is at least about 65%. In some aspects, the sensitivity is at least about 70%. In some aspects, the sensitivity is at least about 75%. In some aspects, the sensitivity is at least about 80%. In some aspects, the sensitivity is at least about 85%. In some aspects, the sensitivity is at least about 90%. In some aspects, the sensitivity is at least about 95%. In some aspects, the ML predictive models for diagnosing and/or predicting the phenotypic impact of a variant (e.g., a missense variant) disclosed herein are clinically significant. In some aspects, the clinical significance of the ML predictive models is determined by the AUC value. In order to be clinically significant, the AUC value is at least about 0.5, at least about 0.55, at least about 0.6, at least about 0.65, at least about 0.7, at least about 0.75, at least about 0.8, at least about 0.85, at least about 0.9, or at least about 0.95. The clinical significance of the predictive models can be determined by the percent accuracy. For example, a predictive model is determined to be clinically significant if the accuracy of the predictive model is at least about 50%, at least about 55%, at least about 60%, at least about 65%, at least about 70%, at least about 72%, at least about 75%, at least about 77%, at least about 80%, at least about 82%, at least about 84%, at least about 86%, at least about 88%, at least about 90%, at least about 92%, at least about 94%, at least about 96%, or at least about 98%. In other aspects, the clinical significance of the ML predictive models for diagnosing and/or predicting the phenotypic impact of a variant (e.g., a missense variant) disclosed herein is determined by the median fold difference (MDF) value. In order to be clinically significant, the MDF value is at least about 0.8, at least about 0.9, at least about 1.0, at least about 1.1, at least about 1.2, at least about 1.3, at least about 1.4, at least about 1.5, at least about 1.6, at least about 1.7, at least about 1.9, or at least about 2.0. In some aspects, the MDF value is greater than or equal to 1.1. In other aspects, the MDF value is greater than or equal to 1.2. Alternatively, or additionally, the clinical significance of the ML predictive models is determined by the t-test P-value. In some aspects, in order to be clinically significant, the t-test P-value is less than about 0.070, less than about 0.065, less than about 0.060, less than about 0.055, less than about 0.050, less than about 0.045, less than about 0.040, less than about 0.035, less than about 0.030, less than about 0.025, less than about 0.020, less than about 0.015, less than about 0.010, less than about 0.005, less than about 0.004, or less than about 0.003. The t-test P-value can be less than about 0.050. Alternatively, the t-test P-value is less than about 0.010. In some aspects, the clinical significance of the ML predictive models for diagnosing and/or predicting the phenotypic impact of a variant (e.g., a missense variant) disclosed herein is determined by the clinical outcome. For example, different clinical outcomes can have different minimum or maximum thresholds for AUC values, MDF values, t-test P-values, and accuracy values that would determine whether the predictive model is clinically significant. In another example, a ML predictive model for diagnosing and/or predicting the phenotypic impact of a variant (e.g., a missense variant) disclosed herein is considered clinically significant if the P-value of the t-test was less than about 0.08, less than about 0.07, less than about 0.06, less than about 0.05, less than about 0.04, less than about 0.03, less than about 0.02, less than about 0.01, less than about 0.005, less than about 0.004, less than about 0.003, less than about 0.002, or less than about 0.001. In some aspects, the performance of the ML predictive models for diagnosing and/or predicting the phenotypic impact of a variant (e.g., a missense variant) disclosed herein is based on the odds ratio. A ML predictive model for diagnosing and/or predicting the phenotypic impact of a variant (e.g., a missense variant) disclosed herein may be considered to have good performance if the odds ratio is at least about 1.30, at least about 1.31, at least about 1.32, at least about 1.33, at least about 1.34, at least about 1.35, at least about 1.36, at least about 1.37, at least about 1.38, at least about 1.39, at least about 1.40, at least about 1.41, at least about 1.42, at least about 1.43, at least about 1.44, at least about 1.45, at least about 1.46, at least about 1.47, at least about 1.48, at least about 1.49, at least about 1.50, at least about 1.52, at least about 1.55, at least about 1.57, at least about 1.60, at least about 1.62, at least about 1.65, at least about 1.67, at least about 1.70 or more. In some aspects, the odds ratio of a ML predictive model is at least about 1.33. The clinical significance of the ML predictive models for diagnosing and/or predicting the phenotypic impact of a variant (e.g., a missense variant) disclosed herein may be based on Univariable Analysis Odds Ratio P-value (uvaORPval). The Univariable Analysis Odds Ratio P-value (uvaORPval) of the predictive model may be between about 0 and about 0.4. The Univariable Analysis Odds Ratio P-value (uvaORPval) of the predictive model may be between about 0 and about 0.3. The Univariable Analysis Odds Ratio P-value (uvaORPval)) of the predictive model may be between about 0 and about 0.2. The Univariable Analysis Odds Ratio P-value (uvaORPval)) of the ML predictive model may be less than or equal to 0.25, less than or equal to about 0.22, less than or equal to about 0.21, less than or equal to about 0.20, less than or equal to about 0.19, less than or equal to about 0.18, less than or equal to about 0.17, less than or equal to about 0.16, less than or equal to about 0.15, less than or equal to about 0.14, less than or equal to about 0.13, less than or equal to about 0.12, or less than or equal to about 0.11. The Univariable Analysis Odds Ratio P-value (uvaORPval) of the a ML predictive model for diagnosing and/or predicting the phenotypic impact of a variant (e.g., a missense variant) disclosed herein may be less than or equal to about 0.10, less than or equal to about 0.09, less than or equal to about 0.08, less than or equal to about 0.07, less than or equal to about 0.06, less than or equal to about 0.05, less than or equal to about 0.04, less than or equal to about 0.03, less than or equal to about 0.02, or less than or equal to about 0.01. The Univariable Analysis Odds Ratio P-value (uvaORPval) of the ML predictive model may be less than or equal to about 0.009, less than or equal to about 0.008, less than or equal to about 0.007, less than or equal to about 0.006, less than or equal to about 0.005, less than or equal to about 0.004, less than or equal to about 0.003, less than or equal to about 0.002, or less than or equal to about 0.001. The clinical significance of the ML predictive models for diagnosing and/or predicting the phenotypic impact of a variant (e.g., a missense variant) disclosed herein may be based on multivariable analysis Odds Ratio P-value (mvaORPval). The multivariable analysis Odds Ratio P-value (mvaORPval)) of the ML predictive model may be between about 0 and about 1. The multivariable analysis Odds Ratio P-value (mvaORPval)) of the ML predictive model may be between about 0 and about 0.9. The multivariable analysis Odds Ratio P-value (mvaORPval)) of the ML predictive model may be between about 0 and about 0.8. The multivariable analysis Odds Ratio P-value (mvaORPval) of the ML predictive model may be less than or equal to about 0.90, less than or equal to about 0.88, less than or equal to about 0.86, less than or equal to about 0.84, less than or equal to about 0.82, or less than or equal to about 0.80. The multivariable analysis Odds Ratio P-value (mvaORPval)) of the ML predictive model may be less than or equal to about 0.78, less than or equal to about 0.76, less than or equal to about 0.74, less than or equal to about 0.72, less than or equal to about 0.70, less than or equal to about 0.68, less than or equal to about 0.66, less than or equal to about 0.64, less than or equal to about 0.62, less than or equal to about 0.60, less than or equal to about 0.58, less than or equal to about 0.56, less than or equal to about 0.54, less than or equal to about 0.52, or less than or equal to about 0.50. The multivariable analysis Odds Ratio P-value (mvaORPval) of the ML predictive model may be less than or equal to about 0.48, less than or equal to about 0.46, less than or equal to about 0.44, less than or equal to about 0.42, less than or equal to about 0.40, less than or equal to about 0.38, less than or equal to about 0.36, less than or equal to about 0.34, less than or equal to about 0.32, less than or equal to about 0.30, less than or equal to about 0.28, less than or equal to about 0.26, less than or equal to about 0.25, less than or equal to about 0.22, less than or equal to about 0.21, less than or equal to about 0.20, less than or equal to about 0.19, less than or equal to about 0.18, less than or equal to about 0.17, less than or equal to about 0.16, less than or equal to about 0.15, less than or equal to about 0.14, less than or equal to about 0.13, less than or equal to about 0.12, or less than or equal to about 0.11. The multivariable analysis Odds Ratio P-value (mvaORPval)) of the ML predictive model may be less than or equal to about 0.10, less than or equal to about 0.09, less than or equal to about 0.08, less than or equal to about 0.07, less than or equal to about 0.06, less than or equal to about 0.05, less than or equal to about 0.04, less than or equal to about 0.03, less than or equal to about 0.02, or less than or equal to about 0.01. The multivariable analysis Odds Ratio P-value (mvaORPval)) of the ML predictive model may be less than or equal to about 0.009, less than or equal to about 0.008, less than or equal to about 0.007, less than or equal to about 0.006, less than or equal to about 0.005, less than or equal to about 0.004, less than or equal to about 0.003, less than or equal to about 0.002, or less than or equal to about 0.001. The clinical significance of the ML predictive models for diagnosing and/or predicting the phenotypic impact of a variant (e.g., a missense variant) disclosed herein may be based on the Kaplan Meier P-value (KM P-value). The Kaplan Meier P-value (KM P-value) of the ML predictive model may be between about 0 and about 0.8. The Kaplan Meier P-value (KM P-value) of the ML predictive model may be between about 0 and about 0.7. The Kaplan Meier P-value (KM P-value) of the ML predictive model and/may be less than or equal to about 0.80, less than or equal to about 0.78, less than or equal to about 0.76, less than or equal to about 0.74, less than or equal to about 0.72, less than or equal to about 0.70, less than or equal to about 0.68, less than or equal to about 0.66, less than or equal to about 0.64, less than or equal to about 0.62, less than or equal to about 0.60, less than or equal to about 0.58, less than or equal to about 0.56, less than or equal to about 0.54, less than or equal to about 0.52, or less than or equal to about 0.50. The Kaplan Meier P-value (KM P-value) of the ML predictive model may be less than or equal to about 0.48, less than or equal to about 0.46, less than or equal to about 0.44, less than or equal to about 0.42, less than or equal to about 0.40, less than or equal to about 0.38, less than or equal to about 0.36, less than or equal to about 0.34, less than or equal to about 0.32, less than or equal to about 0.30, less than or equal to about 0.28, less than or equal to about 0.26, less than or equal to about 0.25, less than or equal to about 0.22, less than or equal to about 0.21, less than or equal to about 0.20, less than or equal to about 0.19, less than or equal to about 0.18, less than or equal to about 0.17, less than or equal to about 0.16, less than or equal to about 0.15, less than or equal to about 0.14, less than or equal to about 0.13, less than or equal to about 0.12, or less than or equal to about 0.11. The Kaplan Meier P-value (KM P-value) of the ML predictive model may be less than or equal to about 0.10, less than or equal to about 0.09, less than or equal to about 0.08, less than or equal to about 0.07, less than or equal to about 0.06, less than or equal to about 0.05, less than or equal to about 0.04, less than or equal to about 0.03, less than or equal to about 0.02, or less than or equal to about 0.01. The Kaplan Meier P-value (KM P-value) of the ML predictive model may be less than or equal to about 0.009, less than or equal to about 0.008, less than or equal to about 0.007, less than or equal to about 0.006, less than or equal to about 0.005, less than or equal to about 0.004, less than or equal to about 0.003, less than or equal to about 0.002, or less than or equal to about 0.001. The clinical significance of the ML predictive models for diagnosing and/or predicting the phenotypic impact of a variant (e.g., a missense variant) disclosed herein may be based on the survival AUC value (survAUC). The survival AUC value (survAUC) of the ML predictive model may be between about 0-1. The survival AUC value (survAUC) of the ML predictive model may be between about 0 to about 0.9. The survival AUC value (survAUC) of the ML predictive model may be less than or equal to about 1, less than or equal to about 0.98, less than or equal to about 0.96, less than or equal to about 0.94, less than or equal to about 0.92, less than or equal to about 0.90, less than or equal to about 0.88, less than or equal to about 0.86, less than or equal to about 0.84, less than or equal to about 0.82, or less than or equal to about 0.80. The survival AUC value (survAUC) of the ML predictive model may be less than or equal to about 0.80, less than or equal to about 0.78, less than or equal to about 0.76, less than or equal to about 0.74, less than or equal to about 0.72, less than or equal to about 0.70, less than or equal to about 0.68, less than or equal to about 0.66, less than or equal to about 0.64, less than or equal to about 0.62, less than or equal to about 0.60, less than or equal to about 0.58, less than or equal to about 0.56, less than or equal to about 0.54, less than or equal to about 0.52, or less than or equal to about 0.50. The survival AUC value (survAUC) of the ML predictive model may be less than or equal to about 0.48, less than or equal to about 0.46, less than or equal to about 0.44, less than or equal to about 0.42, less than or equal to about 0.40, less than or equal to about 0.38, less than or equal to about 0.36, less than or equal to about 0.34, less than or equal to about 0.32, less than or equal to about 0.30, less than or equal to about 0.28, less than or equal to about 0.26, less than or equal to about 0.25, less than or equal to about 0.22, less than or equal to about 0.21, less than or equal to about 0.20, less than or equal to about 0.19, less than or equal to about 0.18, less than or equal to about 0.17, less than or equal to about 0.16, less than or equal to about 0.15, less than or equal to about 0.14, less than or equal to about 0.13, less than or equal to about 0.12, or less than or equal to about 0.11. The survival AUC value (survAUC) of the ML predictive model may be less than or equal to about 0.10, less than or equal to about 0.09, less than or equal to about 0.08, less than or equal to about 0.07, less than or equal to about 0.06, less than or equal to about 0.05, less than or equal to about 0.04, less than or equal to about 0.03, less than or equal to about 0.02, or less than or equal to about 0.01. The survival AUC value (survAUC) of the ML predictive model may be less than or equal to about 0.009, less than or equal to about 0.008, less than or equal to about 0.007, less than or equal to about 0.006, less than or equal to about 0.005, less than or equal to about 0.004, less than or equal to about 0.003, less than or equal to about 0.002, or less than or equal to about 0.001. The clinical significance of the ML predictive models for diagnosing and/or predicting the phenotypic impact of a variant (e.g., a missense variant) disclosed herein may be based on the Univariable Analysis Hazard Ratio P-value (uvaHRPval). The Univariable Analysis Hazard Ratio P-value (uvaRPval) of the ML predictive model may be between about 0 to about 0.4. The Univariable Analysis Hazard Ratio P-value (uvaHRPval) of the ML predictive model may be between about 0 to about 0.3. The Univariable Analysis Hazard Ratio P-value (uvaRPval) of the ML predictive model may be less than or equal to about 0.40, less than or equal to about 0.38, less than or equal to about 0.36, less than or equal to about 0.34, or less than or equal to about 0.32. The Univariable Analysis Hazard Ratio P-value (uvaRPval) of the ML predictive model may be less than or equal to about 0.30, less than or equal to about 0.29, less than or equal to about 0.28, less than or equal to about 0.27, less than or equal to about 0.26, less than or equal to about 0.25, less than or equal to about 0.24, less than or equal to about 0.23, less than or equal to about 0.22, less than or equal to about 0.21, or less than or equal to about 0.20. The Univariable Analysis Hazard Ratio P-value (uvaHRPval) of the ML predictive model may be less than or equal to about 0.19, less than or equal to about 0.18, less than or equal to about 0.17, less than or equal to about 0.16, less than or equal to about 0.15, less than or equal to about 0.14, less than or equal to about 0.13, less than or equal to about 0.12, or less than or equal to about 0.11. The Univariable Analysis Hazard Ratio P-value (uvaHRPval) of the ML predictive model may be less than or equal to about 0.10, less than or equal to about 0.09, less than or equal to about 0.08, less than or equal to about 0.07, less than or equal to about 0.06, less than or equal to about 0.05, less than or equal to about 0.04, less than or equal to about 0.03, less than or equal to about 0.02, or less than or equal to about 0.01. The Univariable Analysis Hazard Ratio P-value (uvaRPval) of the ML predictive model may be less than or equal to about 0.009, less than or equal to about 0.008, less than or equal to about 0.007, less than or equal to about 0.006, less than or equal to about 0.005, less than or equal to about 0.004, less than or equal to about 0.003, less than or equal to about 0.002, or less than or equal to about 0.001. The clinical significance of the ML predictive models for diagnosing and/or predicting the phenotypic impact of a variant (e.g., a missense variant) disclosed herein may be based on the Multivariable Analysis Hazard Ratio P-value (mvaHRPval)mva HRPval. The Multivariable Analysis Hazard Ratio P-value (mvaRPval)mva HRPval of the ML predictive model may be between about 0 to about 1. The Multivariable Analysis Hazard Ratio P-value (mvaHRPval)mva HRPval of the ML predictive model may be between about 0 to about 0.9. The Multivariable Analysis Hazard Ratio P-value (mvaHRPval)mva HRPval of the ML predictive model may be less than or equal to about 1, less than or equal to about 0.98, less than or equal to about 0.96, less than or equal to about 0.94, less than or equal to about 0.92, less than or equal to about 0.90, less than or equal to about 0.88, less than or equal to about 0.86, less than or equal to about 0.84, less than or equal to about 0.82, or less than or equal to about 0.80. The Multivariable Analysis Hazard Ratio P-value (mvaHRPval)mva HRPval of the ML predictive model may be less than or equal to about 0.80, less than or equal to about 0.78, less than or equal to about 0.76, less than or equal to about 0.74, less than or equal to about 0.72, less than or equal to about 0.70, less than or equal to about 0.68, less than or equal to about 0.66, less than or equal to about 0.64, less than or equal to about 0.62, less than or equal to about 0.60, less than or equal to about 0.58, less than or equal to about 0.56, less than or equal to about 0.54, less than or equal to about 0.52, or less than or equal to about 0.50. The Multivariable Analysis Hazard Ratio P-value (mvaHRPval)mva HRPval of the ML predictive model may be less than or equal to about 0.48, less than or equal to about 0.46, less than or equal to about 0.44, less than or equal to about 0.42, less than or equal to about 0.40, less than or equal to about 0.38, less than or equal to about 0.36, less than or equal to about 0.34, less than or equal to about 0.32, less than or equal to about 0.30, less than or equal to about 0.28, less than or equal to about 0.26, less than or equal to about 0.25, less than or equal to about 0.22, less than or equal to about 0.21, less than or equal to about 0.20, less than or equal to about 0.19, less than or equal to about 0.18, less than or equal to about 0.17, less than or equal to about 0.16, less than or equal to about 0.15, less than or equal to about 0.14, less than or equal to about 0.13, less than or equal to about 0.12, or less than or equal to about 0.11. The Multivariable Analysis Hazard Ratio P-value (mvaHRPval)mva HRPval of the ML predictive model may be less than or equal to about 0.10, less than or equal to about 0.09, less than or equal to about 0.08, less than or equal to about 0.07, less than or equal to about 0.06, less than or equal to about 0.05, less than or equal to about 0.04, less than or equal to about 0.03, less than or equal to about 0.02, or less than or equal to about 0.01. The Multivariable Analysis Hazard Ratio P-value (mvaHRPval)mva HRPval of the ML predictive model may be less than or equal to about 0.009, less than or equal to about 0.008, less than or equal to about 0.007, less than or equal to about 0.006, less than or equal to about 0.005, less than or equal to about 0.004, less than or equal to about 0.003, less than or equal to about 0.002, or less than or equal to about 0.001. The clinical significance of the ML predictive models for diagnosing and/or predicting the phenotypic impact of a variant (e.g., a missense variant) disclosed herein may be based on the Multivariable Analysis Hazard Ratio P-value (mvaHRPval). The Multivariable Analysis Hazard Ratio P-value (mvaHRPval) of the predictive model may be between about 0 to about 0.60. Significance of the ML predictive model may be based on the Multivariable Analysis Hazard Ratio P-value (mvaRPval). The Multivariable Analysis Hazard Ratio P-value (mvaRPval) of the ML predictive model may be between about 0 to about 0.50. Significance of the predictive model may be based on the Multivariable Analysis Hazard Ratio P-value (mvaHRPval). The Multivariable Analysis Hazard Ratio P-value (mvaRPval) of the ML predictive model may be less than or equal to about 0.50, less than or equal to about 0.47, less than or equal to about 0.45, less than or equal to about 0.43, less than or equal to about 0.40, less than or equal to about 0.38, less than or equal to about 0.35, less than or equal to about 0.33, less than or equal to about 0.30, less than or equal to about 0.28, less than or equal to about 0.25, less than or equal to about 0.22, less than or equal to about 0.20, less than or equal to about 0.18, less than or equal to about 0.16, less than or equal to about 0.15, less than or equal to about 0.14, less than or equal to about 0.13, less than or equal to about 0.12, less than or equal to about 0.11, or less than or equal to about 0.10. The Multivariable Analysis Hazard Ratio P-value (mvaHRPval) of the ML predictive model may be less than or equal to about 0.10, less than or equal to about 0.09, less than or equal to about 0.08, less than or equal to about 0.07, less than or equal to about 0.06, less than or equal to about 0.05, less than or equal to about 0.04, less than or equal to about 0.03, less than or equal to about 0.02, or less than or equal to about 0.01. The Multivariable Analysis Hazard Ratio P-value (mvaHRPval) of the ML predictive model may be less than or equal to about 0.01, less than or equal to about 0.009, less than or equal to about 0.008, less than or equal to about 0.007, less than or equal to about 0.006, less than or equal to about 0.005, less than or equal to about 0.004, less than or equal to about 0.003, less than or equal to about 0.002, or less than or equal to about 0.001. The performance of the ML predictive models for diagnosing and/or predicting the phenotypic impact of a variant (e.g., a missense variant) disclosed herein may be determined by AUC values and an improvement in performance may be determined by the difference in the AUC value of the ML predictive model disclosed herein and the AUC value of current predictive models. In some aspects, a ML predictive model disclosed herein outperforms current predictive models when the AUC value of the ML predictive model disclosed herein is greater than the AUC value of the current predictive models by at least about 0.05, by at least about 0.06, by at least about 0.07, by at least about 0.08, by at least about 0.09, by at least about 0.10, by at least about 0.11, by at least about 0.12, by at least about 0.13, by at least about 0.14, by at least about 0.15, by at least about 0.16, by at least about 0.17, by at least about 0.18, by at least about 0.19, by at least about 0.20, by at least about 0.022, by at least about 0.25, by at least about 0.27, by at least about 0.30, by at least about 0.32, by at least about 0.35, by at least about 0.37, by at least about 0.40, by at least about 0.42, by at least about 0.45, by at least about 0.47, or by at least about 0.50 or more. In some aspects, the AUC value of the ML predictive model disclosed herein is greater than the AUC value of the current predictive models by at least about 0.10. In some aspects, the AUC value of the ML predictive model disclosed herein is greater than the AUC value of the current predictive models by at least about 0.13. In some aspects, the AUC value of the ML predictive model disclosed herein is greater than the AUC value of the current predictive models by at least about 0.18. The performance of the ML predictive models for diagnosing and/or predicting the phenotypic impact of a variant (e.g., a missense variant) disclosed herein may be determined by the odds ratios and an improvement in performance may be determined by comparing the odds ratio of the ML predictive model disclosed herein and the odds ratio of current predictive models. Comparison of the performance of two or more predictive models can generally be based on the comparison of the absolute value of (1-odds ratio) of a first predictive model to the absolute value of (1-odds ratio) of a second predictive model. Generally, the predictive model with the greater absolute value of (1-odds ratio) can be considered to have better performance as compared to the predictive model with a smaller absolute value of (1-odds ratio).

In some aspects, the performance of a ML predictive model for diagnosing and/or predicting the phenotypic impact of a variant (e.g., a missense variant) disclosed herein is based on the comparison of the odds ratio and the 95% confidence interval (CI). For example, a first predictive model may have a greater absolute value of (1-odds ratio) than a second predictive model, however, the 95% CI of the first predictive model may overlap 1 (e.g., poor accuracy), whereas the 95% CI of the second predictive model does not overlap 1. In this instance, the second predictive model is considered to outperform the first predictive model because the accuracy of the first predictive model is less than the accuracy of the second predictive model. In another example, a first predictive model may outperform a second predictive model based on a comparison of the odds ratio; however, the difference in the 95% CI of the first predictive model is at least about 2 times greater than the 95% CI of the second predictive model. In this instance, the second predictive model is considered to outperform the first predictive model

In some aspects, a ML predictive model for diagnosing and/or predicting the phenotypic impact of a variant (e.g., a missense variant) disclosed herein is more accurate than a current predictive model. The ML predictive model disclosed herein is more accurate than a current predictive model if the range of 95% CI of the ML predictive model disclosed herein does not span or overlap 1 and the range of the 95% CI of the current predictive model or clinical variable spans or overlaps 1. In some aspects, a ML predictive model for diagnosing and/or predicting the phenotypic impact of a variant (e.g., missense variant) disclosed herein is more accurate than a current predictive model known in the art. In some aspects, the ML predictive model disclosed herein is more accurate than a current predictive model known in the art when the difference in range of the 95% CI of the ML predictive model disclosed herein is about 0.70, about 0.60, about 0.50, about 0.40, about 0.30, about 0.20, about 0.15, about 0.14, about 0.13, about 0.12, about 0.10, about 0.09, about 0.08, about 0.07, about 0.06, about 0.05, about 0.04, about 0.03, or about 0.02 times less than the difference in range of the 95% CI of the current predictive model. The ML predictive model disclosed herein is more accurate than a current predictive model when the difference in range of the 95% CI of the ML predictive model disclosed herein is between about 0.20 to about 0.04 times less than the difference in range of the 95% CI of the current predictive model.

### EXAMPLES

### Example 1

### Predictive Model for the

### MSH2 (MutS Homolog 2)/MSH6 (MutS Homolog 6) Heterodimer

In one specific example, the native MSH2/MSH6 heterodimer was simulated using MD to generate conformational dynamics that were then used to determine the native conformational landscape. From the conformational dynamics a low dimensional representation was determined that was used to identify 30 metastable conformational state conformations. See FIG. 6. From each of these conformations, 45 variants were introduced to the MSH2 protein, and simulations were performed for each variant. Each variant was simulated 10 times in each of the 30 states for between 1 and 25 ns, totaling less than 1 microsecond per variant. In comparison, the native MSH2/MSH6 heterodimer was simulated for −5 microseconds to determine the conformational landscape.

Each variant simulation was processed to reduce dimensionality by first extracting structural features (either the backbone dihedral angles (phi, psi angles), the connectedness of each amino acid position as defined by the number of nearby amino acids with 15 nm, or the inter-residue distance of amino acid pairs that are close than 18 nm) for each conformation in each trajectory in each state. Then the dimensionality of the data was further reduced using PCA or tICA to a final dimensionality of 64 features per state.

For model training, how much trajectory data would be used in modeling to determine the minimum amount of simulation time needed was tested. Three dataset definitions were used: 10 ns of one repetition for 4 states (40 ns per variant), 5 ns of one repetition of 17 states (85 ns per variant) or 3 ns of two repetitions for 11 states (66 ns per variant). See FIG. 8. This represented a dramatic decrease in simulation time required per variant compared to the −5 microseconds of simulation required to determine the conformational landscape of the native MSH2/MSH6 heterodimer.

Together, the five dimensionality reduction methods (dihedral angles, tlCA; connectedness, tlCA; dihedral angles, PCA; contact, PCA; and connectedness, PCA) and the three dataset definitions (10 ns, 1 repeat, 4 states; 5 ns, 1 repeat, 17 states; 3 ns, 2 repeats, 11 states) were used for hyperparameter tuning of the featurization method and dataset definition.

The reduced dimensionality data was then modeled using sklearn pipelines to do hyperparameter tuning. Twenty-five percent of the data was set aside as a test-set and the remaining data was split into 5 fold cross-validation sets. The cross-validation set was trained using a random forest model, hyper-parameter tuning the number of estimators, the minimum number of samples to split a node, the maximum number of features per tree, the maximum depth of the trees and minimum samples per leaf. The hyperparameter tuning scored on the F1 accuracy score. The cross-validation set performance was used to evaluate the featurization method and dataset definition. The hold-out test set was used to evaluate the generalization of the model after selecting the top model based on cross-validation performance.

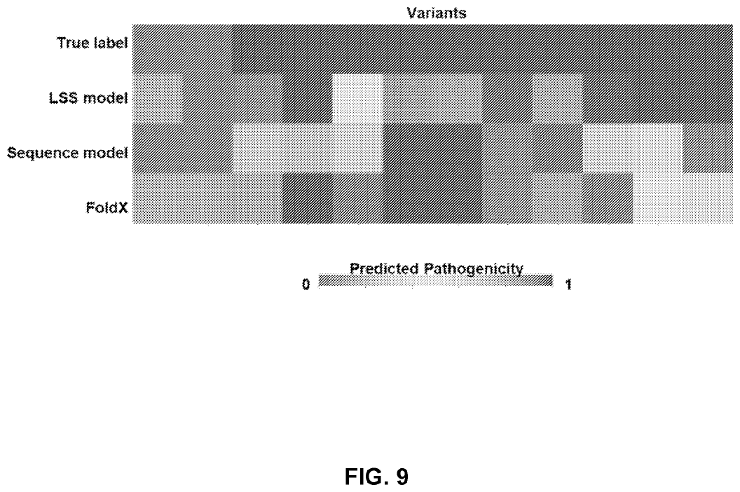

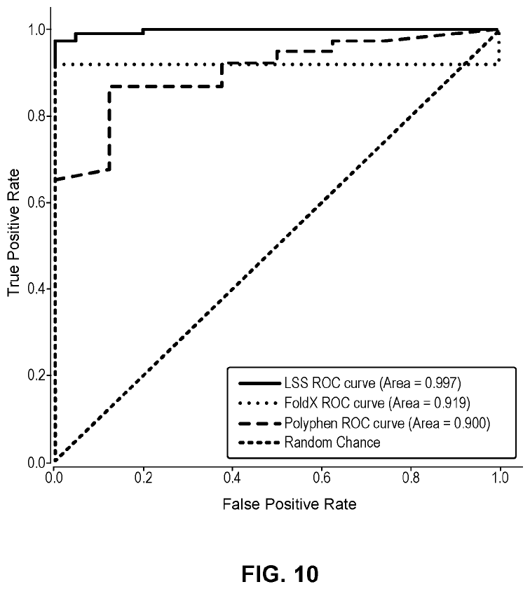

From the featurization methods, the dataset definition and model hyperparameter tuning, high performance accuracy (>0.8) was achieved for nearly all combinations. As shown in FIG. 9, when the performance of the best performing model (LSS model), generated according to the methods of the present disclose, over a selection of variants was compared to the performance of the same variants in a sequence-based model and a FoldX-based model and compared to the true label, overall the LSS model predictions for these variants were closer to the true labels. The best performing model (LSS model) from the cross-validation set (using the amino acid connectedness, tICA dimensionality reduction) achieved a model performance on the hold-out test set that was better than the performance of sequence-based and FoldX based models. As shown in FIG. 10 the performance of the predictive model for the MSH2/MSH6 heterodimer developed according to the methods disclosed herein (LSS model) performed better than alternative methods developed using, e.g., FoldX, Polyphen, or process-scikit (an INVITAE™-developed computational predictor), as measured by the area under a Receiver Operating Characteristic (ROC) curve. The LSS model achieves a model performance of 0.997 AUC, whereas FoldX, Polyphen, and Process-scikit achieved performances of 0.919, 0.900 and 0.900 AUC, respectively.

### VI. Terms

In order that the present disclosure can be more readily understood, certain terms are first defined. As used in this disclosure, except as otherwise expressly provided herein, each of the following terms shall have the meaning set forth below. Additional definitions are set forth throughout the disclosure.

The singular forms “a”, “an” and “the” include plural referents unless the context clearly dictates otherwise. The terms “a” (or “an”), as well as the terms “one or more,” and “at least one” can be used interchangeably herein. In certain aspects, the terms “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As described herein, any concentration range, percentage range, ratio range or integer range is to be understood to include the value of any integer within the recited range and, when appropriate, fractions thereof (such as one tenth and one hundredth of an integer), unless otherwise indicated.

It is understood that wherever aspects are described herein with the language “comprising,” otherwise analogous aspects described in terms of “consisting of” and/or “consisting essentially of” are also provided.

Units, prefixes, and symbols are denoted in their Système International d'Unités (SI) accepted form. The headings provided herein are not limitations of the various aspects of the disclosure, which can be had by reference to the specification as a whole. Accordingly, the terms defined are more fully defined by reference to the specification in its entirety.

The terms “about,” “comprising essentially of,” or “consisting essentially of,” refer to a value or composition that is within an acceptable error range for the particular value or composition as determined by one of ordinary skill in the art, which will depend in part on how the value or composition is measured or determined, i.e., the limitations of the measurement system. For example, “about,” “comprising essentially of,” or “consisting essentially of,” can mean within 1 or more than 1 standard deviation per the practice in the art. Alternatively, “about,” “comprising essentially of,” or “consisting essentially of,” can mean a range of up to 10%. Furthermore, particularly with respect to biological systems or processes, the terms can mean up to an order of magnitude or up to 5-fold of a value. When particular values or compositions are provided in the specification and claims, unless otherwise stated, the meaning of “about,” “comprising essentially of,” or “consisting essentially of,” should be assumed to be within an acceptable error range for that particular value or composition.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

It is to be appreciated that the Detailed Description section, and not the Summary and Abstract sections, is intended to be used to interpret the embodiments. The Summary and Abstract sections can set forth one or more but not all exemplary embodiments of the present invention as contemplated by the inventor(s), and thus, are not intended to limit the present invention and the appended embodiments in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following embodiments and their equivalents.

The contents of all cited references (including literature references, patents, patent applications, and websites) that may be cited throughout this disclosure are hereby expressly incorporated by reference in their entirety for any purpose, as are the references cited therein.

What is claimed is:

1. A computer implemented method for high throughput classification of variant effects comprising
(i) generating conformational dynamics (e.g. using molecular dynamics (MD)) of a reference molecule structure;
(ii) processing the conformational dynamics of the reference molecule structure to generate a low-dimensional representation of the conformational landscape of the reference molecule structure;
(iii) extracting a metastable conformational state from the low-dimensional conformational landscape of the reference molecule structure to seed simulations of a variant molecule structure;
(iv) generating a variant molecule structure or set thereof by introducing at least one variant in the extracted conformational state of the reference molecule structure;
(v) generating conformational dynamics of the variant molecule structure or set thereof (e.g. using MD);
(vi) processing the conformational dynamics of the variant molecule structure or set thereof to generate a low-dimensional representation of the conformational landscape of the variant molecule structure or set thereof; and,
(vii) training a machine-learning predictive model using the low-dimensional representation of the conformational landscape of the reference molecule structure and variant molecule structures using clinical data as training labels, wherein the predictive model classifies the variant effects in the reference molecule structure.

2. The method claim 1, wherein generating conformational dynamics of the reference molecule structure or variant molecule structure comprises
(a) retrieving a reference molecule structure or variant molecule structure; and,
(b) generating conformational dynamics using the retrieved reference molecule structure or variant protein structure.

3. The method of claim 2, wherein the reference molecule structure or variant molecule structure are retrieved from the Protein Data Bank (PDB) or AlphaFold.

4. The method of claim 2, wherein the conformational dynamics are generated using GROMACS, OpenMM, NAMD, Amber or LAMPS.

5. The method of claim 2, wherein the generation of conformational dynamics is parallelized.

6. The method of claim 1, wherein the reference molecule structure is a three dimensional structure of a wild type protein.

7. The method of any one of claims 1 to 6, wherein each one of step (i) and/or step (ii) of claim 1 comprises independently at least about 10,000, at least about 20,000, at least about 30,000, at least about 40,000, at least about 50,000, at least about 60,000, at least about 70,000, at least about 80,000, at least about 90,000, at least about 100,000, at least about 200,000, at least about 300,000, at least about 400,000, at least about 500,000, at least about 600,000, at least about 700,000, at least about 800,000, at least about 900,000, or at least about 1,000,000 simulated structural conformations that comprise the conformational dynamics.

8. The method of claim 1, wherein processing the conformational dynamics to generate a low-dimensional representation of the conformational landscape of the reference molecule structure comprises extracting structural features from the conformational dynamics.

9. The method of claim 8, wherein the structural features are selected from the group consisting of bond angles, inter-residue distances, residue fluctuation, surface accessibility, domain relative orientations, binding-site interactions, and any combination thereof.

10. The method of claims 8 or 9, wherein the extraction of structural features results in at least a 20-fold, 30-fold, 40-fold, or 50-fold reduction in structural features.

11. The method of any one of claims 8 to 10, wherein processing the conformational dynamics further comprises dimensionality reduction.

12. The method of claim 11, wherein the dimensionality reduction is obtained using principal component analysis (PCA) or autoencoders.

13. The method of claim 11 or 12, wherein the dimensionality reduction results in at least a 50-fold, 60-fold, 70-fold, 80-fold, 90-fold or 100-fold reduction in structural features.

14. The method of any one of claims 8 to 13, wherein the low-dimensional representation of the conformational landscape identifies conformations in which the reference molecule structure and/or variant molecule structure is in a metastable conformational state.

15. The method of claim 1, wherein the low-dimensional representation comprises modes of movement that characterize conformational changes that are relevant to molecular function.

16. The method of any one of claims 1 to 16, wherein extracting a metastable conformational state from the low-dimensional conformational landscape comprises clustering conformational states based on their representation in the low-dimensional space.

17. The method of any one of claim 16, wherein clustering conformational states comprises k-mean clustering, hierarchical clustering, density-based clustering, DBSCAN, spectral clustering, Gaussian mixture models, or any combination thereof.

18. The method of any one of claims 16 or 17, further comprising prioritizing the clustered conformational states.

19. The method of claim 18, wherein prioritizing clustered conformational states is based on cluster properties.

20. The method of claim 19, wherein the cluster properties are selected from the group consisting of distance from conformational landscape centroid, cluster occupancy, cluster distribution statistics, structural properties of the cluster, thermodynamics of cluster, or any combination thereof.

21. The method of claim 20, wherein the structural properties are selected from the group consisting of bond angles, inter-residue distances, surface accessibility, domain relative orientations, binding-site interactions, structural similarity to known protein conformations, and any combination thereof.

22. The method of claim 21, wherein the bond angles comprise phi-psi dihedrals, side chain chi angles, or combinations thereof.

23. The method of claim 20, wherein the cluster distribution statistics are selected from the group consisting of Silhouette score, elbow score, Calinski-Harabasz Index, Rand Index, mutual information, homogeneity, completeness, V measure, Davies-Bouldin Index, and any combination thereof.

24. The method of any one of claims 1 to 23, wherein extracting a metastable conformational state from the low-dimensional conformational landscape comprises extracting a representative conformational state.

25. The method of claim 24, wherein the representative conformational state is a conformational state that is amongst the closest to the centroid of the all the conformational states of a cluster.

26. The method of claim 1, wherein at least one variant is a pathogenic mutation.

27. The method of claim 1, wherein at least one variant is a non-pathogenic (benign) mutation.

28. The method of claim 1, wherein the variant molecule structure or set thereof comprises a variant observed or that could be observed in medical genetic testing.

29. The method of claim 1, wherein the variant molecule structure or set thereof comprises a pathogenic variant.

30. The method of claim 1, wherein the variant molecule structure or set thereof comprises a non-pathogenic (benign) variant.

31. The method of claim 1, wherein the variant molecule structure or set thereof comprises a variant of unknown significance.

32. The method of claim 1, wherein the variant molecule structure or set thereof is generated using a backbone-dependent rotamer library.

33. The method of claim 32, comprising identifying sidechain conformations (rotamers) with the existing backbone dihedral angles of the variant molecule structure at the variant position in the backbone-dependent rotamer library.

34. The method of claim 33, further comprising testing whether the introduction of a rotamer identified backbone-dependent rotamer library causes a steric clash with nearby residues.

35. The method of claim 34, wherein testing is conducted until a rotamer is identified that minimizes steric clash.

36. The method of claim 34, further comprising conducting a minimization and equilibration simulation to regularize the geometry of the conformation of the variant molecule structure and decrease the energetic impact of introducing the variant in the variant molecule structure.

37. The method of claim 1, wherein the predictive model is generated using machine-learning.

38. The method of claim 1, wherein the clinical data used as training labels comprises biomarker status (e.g., presence or absence of a certain biomarker or its expression level), biometric data, lifestyle-related data, response to treatments, symptoms of the disease or conditions, protein expression data, type of treatment administered, dosage, dosage regimen, administration route, presence or absence of co-therapies, response to the therapy, age, body weight, gender, ethnicity, ClinVar submissions, INVITAE™ clinical interpretations, data from other experimental or computational models, or any combination thereof.

39. The method of claim 1, wherein the classification of the variant effect in the reference molecule structure comprises the calculation of a predicted pathogenicity probability.

40. The method of claim 1, wherein the machine-learning predictive model is generated using Logistic Regression.

41. The method of claim 1, wherein the machine-learning predictive model is generated using Random Forests.

42. The method of claim 1, wherein the machine-learning predictive model is generated using an Artificial Neural Network.

43. A computer implemented method for scoring the clinical significance of a variant comprising:
(a) generating conformational dynamics (e.g., using MD) of a reference molecule structure;
(b) processing the conformational dynamics of the reference molecule structure to generate a low-dimensional representation of the conformational landscape of the reference molecule structure;
(c) extracting a metastable conformational state from the low-dimensional conformational landscape of the reference molecule structure to seed simulations of a variant molecule structure;
(d) generating a variant molecule structure or set thereof by introducing at least one variant in the extracted metastable conformational state of the reference molecule structure;
(e) generating conformational dynamics of the variant molecule structure or set thereof (e.g. using MD);
(f) processing the conformational dynamics of the variant molecule structure or set thereof to generate a low-dimensional representation of the conformational landscape of the variant molecule structure or set thereof; and,
(g) training a machine-learning predictive model using the low-dimensional representation of the conformational landscape of the reference molecule structure and variant molecule structures using clinical data as training labels, wherein the predictive model outputs a score the clinical significance of the variant.

44. A system comprising
(i) a memory; and,
(ii) at least one processor coupled to the memory and configured for
i. generating conformational dynamics of a reference molecule structure (e.g. using MD);
ii. processing the conformational dynamics of the reference molecule structure to generate a low-dimensional representation of the conformational landscape of the reference molecule structure;
iii. extracting a metastable conformational state from the low-dimensional conformational landscape of the reference molecule structure to seed simulations of a variant molecule structure;
iv. generating a variant molecule structure or set thereof by introducing at least one variant in the extracted metastable conformational state of the reference molecule structure;
v. generating conformational dynamics of the variant molecule structure or set thereof (e.g. using MD);
vi. processing the conformational dynamics of the variant molecule structure or set thereof to generate a low-dimensional representation of the conformational landscape of the variant molecule structure or set thereof; and,
vii. training a machine-learning predictive model using the low-dimensional representation of the conformational landscape of the reference molecule structure and variant molecule structures using clinical data as training labels, wherein the predictive model classifies the variant effects in the reference molecule structure.

45. A tangible computer readable device having instruction stored thereon that, when executed by at least one computing device, causes the at least one computing device to perform operations comprising:
i. generating conformational dynamics (e.g. using MD) of a reference molecule structure;
ii. processing the conformational dynamics of the reference molecule structure to generate a low-dimensional representation of the conformational landscape of the reference molecule structure;
iii. extracting a metastable conformational state from the low-dimensional conformational landscape of the reference molecule structure to seed simulations of a variant molecule structure;
iv. generating a variant molecule structure or set thereof by introducing at least one variant in the extracted metastable conformational state of the reference protein structure;
v. generating conformational dynamics of the variant molecule structure or set thereof (e.g. using MD);
vi. processing the conformational dynamics of the variant molecule structure or set thereof to generate a low-dimensional representation of the conformational landscape of the variant molecule structure or set thereof; and,
vii. training a machine-learning predictive model using the low-dimensional representation of the conformational landscape of the reference molecule structure and variant molecule structures using clinical data as training labels, wherein the predictive model classifies the variant effects in the reference molecule structure.

46. The method of any one of claims 1 to 43, system of claim 44, or tangible computable readable device of claim 45 wherein the classification of the variant in the variant molecule structure can be used to (i) treat a patient, (ii) selected a patient for treatment, (iii) commence a treatment, (iv) discontinue a treatment, (v) interrupt a treatment, (vi) modify a treatment, or (vii) any combination thereof.

47. A method to (i) treat a patient, (ii) selected a patient for treatment, (iii) commence a treatment, (iv) discontinue a treatment, (v) interrupt a treatment, (vi) modify a treatment, or (vii) any combination thereof, comprising applying the method of any one of claims 1 to 43, system of claim 44, or tangible computable readable device of claim 45.

48. A personalized medicine treatment comprising a therapeutic agent that can treat a disease or disorder caused by a variant identified as pathogenic by the method of any one of claims 1 to 43, or by using the system of claim 44, or the tangible computable readable device of claim 45.

49. A computer implemented method for generating a low-dimensional representation of the conformation landscape of a molecule structure comprising (i) extracting structural features from the conformational landscape of the molecule structure, and (ii) reducing the dimensionality of the conformational landscape.

50. The method of claim 49 wherein the structural features are selected from the group consisting of bond angles, inter-residue distances, residue fluctuation, surface accessibility, domain relative orientations, binding-site interactions, and any combination thereof.

51. The method of claim 50, wherein extracting structural features results in at least a 20-fold, 30-fold, 40-fold, or 50-fold reduction in structural features.

52. The method of claim 49, wherein reducing the dimensionality comprises using principal component analysis (PCA) or autoencoders.

53. The method of claim 49, wherein reducing the dimensionality results in at least a 50-fold, 60-fold, 70-fold, 80-fold, 90-fold or 100-fold reduction in structural features.

54. A computer implemented method for identifying metastable conformational states of a molecule structure comprising clustering low-dimensional representations of the conformational landscape of the molecule structure.

55. The method of claim 54, wherein clustering conformational states comprises k-mean clustering, hierarchical clustering, density-based clustering, DBSCAN, spectral clustering, Gaussian mixture models, or any combination thereof.

56. The method of claims 54 or 55, further comprising prioritizing the clustered conformational states.

57. The method of claim 56, wherein prioritizing clustered conformational states is based on cluster properties.

58. The method of claim 57, wherein the cluster properties are selected from the group consisting of distance from conformational landscape centroid, cluster occupancy, cluster distribution statistics, structural properties of the cluster, thermodynamics of cluster, or any combination thereof.

59. The method of claim 58, wherein the structural properties are selected from the group consisting of bond angles, inter-residue distances, surface accessibility, domain relative orientations, binding-site interactions, structural similarity to known protein conformations, and any combination thereof.

60. The method of claim 59, wherein the bond angles comprise phi-psi dihedrals, side chain chi angles, or combinations thereof.

61. The method of claim 58, wherein the cluster distribution statistics are selected from the group consisting of Silhouette score, elbow score, Calinski-Harabasz Index, Rand Index, mutual information, homogeneity, completeness, V measure, Davies-Bouldin Index, and any combination thereof.

62. A computer implemented method for high throughput classification of variant effects in a molecule structure comprising (i) generating a low-dimensional representation of the conformation landscape of the molecule structure, and (ii) identifying metastable conformational states from the low-dimensional representation of the conformation landscape of the molecule structure.

63. The method of claim 62, wherein generating a low-dimensional representation of the conformation landscape of the molecule comprises (a) extracting structural features from the conformation landscape of the molecule structure, and (b) reducing the dimensionality of the conformational landscape.

64. The method of claim 62, wherein identifying metastable conformational states from the low-dimensional representation of the conformation landscape of the molecule structure comprises clustering low-dimensional representations of the conformation landscape of the molecule structure.
